(19) United States
(12) Patent Application Publication
(10) Pub. No.: US 2024/0379248 A1
(43) Pub. Date: Nov. 14, 2024

(54) MACHINE LEARNING FOR DESIGNING ANTIBODIES AND NANOBODIES IN-SILICO

(71) Applicant: MarWell Bio Inc., South San Francisco, CA (US)

(72) Inventors: Zara Hemmatian, Half Moon Bay, CA (US); Adrian Alireza Chavosh, Half Moon Bay, CA (US)

(21) Appl. No.: 18/615,028

(22) Filed: Mar. 25, 2024

Related U.S. Application Data
(63) Continuation of application No. PCT/US2022/044754, filed on Sep. 26, 2022.
(60) Provisional application No. 63/248,761, filed on Sep. 27, 2021.
(60) Provisional application No. 63/318,037, filed on Mar. 9, 2022.
(60) Provisional application No. 63/332,418, filed on Apr. 19, 2022.
(60) Provisional application No. 63/395,487, filed on Aug. 5, 2022.
(60) Provisional application No. 63/397,603, filed on Aug. 12, 2022.

(51) Int. Cl. G16H 70/40

(52) U.S. Cl. CPC G16H 70/40

(57) ABSTRACT

A computer-implemented method for generating a set of candidate variant amino acid sequences of an antibody, a nanobody, or a fragment thereof, having binding ability to a target protein, may comprise: (a) obtaining a set of seed amino acid sequences; and (b) processing the set of seed amino acid sequences using a first trained machine learning algorithm to generate the set of candidate amino acid sequences, wherein the first trained machine learning algorithm is trained with first training data comprising a set of training amino acid sequences for the target protein, wherein the first trained machine learning algorithm is further trained through a transfer learning method using a second trained machine learning algorithm, wherein the second trained machine learning algorithm is trained with second training data comprising a set of training amino acid sequences for a second target protein, wherein the second target protein is different from the target protein.

### CROSS-REFERENCE

This application is a continuation of International Application No. PCT/US2022/044754, filed Sep. 26, 2022, which claims the benefit of U.S. Provisional Application No. 63/248,761, filed Sep. 27, 2021, U.S. Provisional Application No. 63/318,037, filed Mar. 9, 2022, U.S. Provisional Application No. 63/332,418, filed Apr. 19, 2022, U.S. Provisional Application No. 63/395,487, filed Aug. 5, 2022, and U.S. Provisional Application No. 63/397,603, filed Aug. 12, 2022, each of which is incorporated by reference herein in its entirety.

### BACKGROUND

Antibodies play an important role in therapeutics discovery and vaccine manufacturing due to their ability to bind to one and/or more target antigens. The discovery and generation of antibodies and nanobodies may be performed via in-silico methods.

### SUMMARY

The binding site of an antibody/nanobody may include a region referred to as the complementarity-determining region (CDR). Amongst CDRs, the third complementarity-determining region (CDR) of the heavy chain (CDR-H3) of an antibody/nanobody and/or fragment may be the region of highest sequence diversity, which may play a significant role in antigen recognition and dictates binding specificity and affinity. A deep neural network capable of learning from complex and highly variant sequences may be developed to model CDR-H3 sequences and/or to design CDR-H3 sequences mutations. However, increasing the depth of the neural network by adding more layers may lead to challenges such as overfitting and vanishing gradient problems. Moreover, existing deep learning approaches may need to be trained with large training datasets, and may be incapable of discovering and generating new antibodies/nanobodies and/or fragments when facing data scarcity for novel targets. In addition, there are a number of key therapeutics and developability properties to screen for identifying the lead antibody/nanobody candidates. The key therapeutics and developability properties may include but are not limited to antibody specificity to one or multiple targets, cross-reactivity to one or multiple targets, viscosity, clearance, stability, solubility, affinity, affinity maturation, epitope mapping, yield, aggregation, function immunogenicity, humanization, and humaneness. Therefore, there remains a need for tools that can rapidly analyze antibodies/nanobodies and/or fragments for their therapeutic and developability properties, in order to identify the lead candidates, perform analysis of the antibodies/nanobodies for their key biophysical and therapeutic properties, such as specificity and cross-reactivity, and/or repurpose the antibodies/nanobodies for different targets.

In addition, accurate prediction of antibody-antigen binding affinity requires time-consuming and expensive wet-lab experimentations. Therefore, a deep machine-learning method that can rapidly assess the antibody-antigen binding landscape is of significant importance.

Furthermore, besides the CDR-H3, the variable regions (Fv) in antibodies/nanobodies play a significant role in their specificity and binding affinity. However, existing artificial intelligence (AI) methods for antibody discovery mainly focus on antibodies' CDR-H3 region and not on the antibodies' variable fragment (Fv) regions. Thus, a deep machine-learning method that can predict antibody variable regions (Fvs), humanized Fvs, and antibody variable region mutations is of substantial importance.

Moreover, an essential task in antibody therapeutics discovery is to rapidly identify whether an antibody can recognize a specific epitope on an antigen surface and bind to it. Characterization of antibody-paratope:antigen-epitope interaction provides valuable mechanistic insights that can enhance antibody therapeutics' effectiveness. However, other methods may be overly costly and labor-intensive as repetitive wet-lab experimentations are required to sufficiently explore the antibody sequence space. Moreover, due to the extended time and the significant cost associated with those methods, such analysis may be used at the late stage of the discovery and development process and on a limited number of antibody candidates or antigenic variants. This reduces the number of goal shots among the antibody sequences galaxy and increases the risk of failure. Therefore, a deep machine-learning method that can enable an early stage, high-throughput, and efficient method for rapid and accurate antibody analysis, which relies on only amino acid sequences is of critical importance.

Moreover, the design of new therapeutic antibodies requires accurate paratope prediction for optimized antibody paratope-antigen epitope interactions that can be achieved through 3D structure modeling. In-silico antibody discovery for novel targets begins with antibody amino acid sequence data without antibody 3D structure data. The accurate modeling of antigen epitope-antibody paratope requires the model to incorporate the antigen epitope 3D structure to predict the antibody paratope 3D structure. Moreover, besides the backbone residue region in the antibody paratope, the surface side-chain conformation of the antibody paratope plays an important role in the binding interaction of the antibody paratope-antigen epitope. Therefore, the method may need to model both surface side chains and backbone residues in predicting antibody paratope 3D structure. However, the existing artificial intelligence (AI) methods developed for designing new antibody therapeutics do not incorporate the antigen 3D structure and do not model the surface side-chain conformation. Thus, there is a need for rapid and accurate antibody paratope design by the autoregressive generation of antibody paratope sequences while recursively improving antibody paratopes' backbones and side chain structures.

Described herein are systems and techniques for precisely designing and developing antibodies/nanobodies for existing and/or new targets. In some embodiments, the techniques may be used to design and develop antibodies/nanobodies and/or fragments against one target. In other embodiments, the techniques may be used to design and develop antibodies/nanobodies and/or fragments against multiple targets.

In some aspects, the present disclosure provides a deep learning-based antibody design tool. The antibody design tool may include but is not limited to a variational autoencoder (VAE) and residual neural network (Resnet), VAE-Resnet-based algorithm equipped with a transfer learning technique that generates antibody CDR3 sequences (e.g., particularly for novel and difficult targets for which the training data is often scarce), a generative adversarial network (GAN) and reinforcement learning (RL) GAN-RL-based algorithm that generates humanized antibody variable regions, and a conditioned deep learning graph neural network (GNN)-based 3D structure predictor that accurately designs antibodies while predicting antibody paratopes' 3D structures.

In some aspects, the present disclosure provides a deep learning-based antibody analysis tool. The antibody analysis tool may include but is not limited to a VAE-Resnet-based algorithm that reduces the dimensionality of antibodies by extracting the key features, analyzes antibodies for their key biological and key therapeutics (e.g., specificity and cross-reactivity), repurposes existing antibodies for other targets, and improves the performance of clustering models; a deep learning-based epitope mapping algorithm that predicts the epitopes using only amino acid sequences for early stage antibody assessment and for accurately identifying the antibody's mechanism of action; a deep learning-based classifier that accurately classifies the generated antibodies based on their binding ability to a given target; BioOne-Hot preprocessing for improving the antibody classification; and an AI-based automated and multimodal screening method to identify a short list of the best-in-class antibodies for their key therapeutic and biophysical properties.

A deep machine-learning engine is developed and trained using a variety of antibodies/nanobodies known to their targets. In some embodiments, the targets may include but are not limited to antigens. In other embodiments, the deep machine-learning engine may predict antibodies/nanobodies sequences for new target antigens that were and/or were not among the antibodies/nanobodies sequences used for training. In other embodiments, a deep learning model may be used to perform epitope mapping/epitope prediction at the early stage of discovery using only amino acid sequences. In another embodiment, the deep machine-learning engine utilizes a given antigen epitope data to predict the 3D structure of the antibody/nanobody paratopes that bind to the given antigen epitope. The deep machine-learning engine predicts the antibody/nanobody paratope 3D structure by modeling the antigen epitope-antibody paratope interactions. The predicted antibody/nanobody paratope 3D structure comprises the backbone residue region and the surface side chains. In other embodiments, the deep machine-learning engine may predict the antibody paratope binding affinity by modeling the antigen epitope-antibody paratope interactions. In other embodiments, the deep machine-learning method may employ transfer-learning techniques to design antibodies/nanobodies for a novel target lacking sufficient training data. In some embodiments, the techniques may be used to design antibodies/nanobodies and/or antibodies/nanobodies mutations and/or to screen their biophysical properties. In another embodiment, the biophysical properties include but are not limited to net-charge, hydrophobicity, viscosity, clearance, solubility, stability, binding affinity, affinity maturation, isoelectric points, specificity, cross-reactivity, immunogenicity, humaneness, humanization, developability, manufacturability, half-life, pharmacokinetic, yield, aggregation, function, and antibody epitope mapping to identify the lead candidates for their therapeutics developability. In other embodiments, the techniques may be used for automated and multimodal screening to identify a short list of the best-in-class antibodies for their key therapeutic and biophysical properties. The deep-learning engine can reduce dimensions in the antibodies/nanobodies sequences data, and/or extracts the key biophysical features and/or group antibodies/nanobodies sequence data into clusters and/or improve the clustering of antibodies/nanobodies sequences and/or recognize hidden patterns across the clusters in their latent spaces and/or repurpose the antibodies/nanobodies sequences recognized from the hidden patterns from one target to be used against multiple targets. In some embodiments, the deep machine-learning engine can recognize antibodies/nanobodies having specificity to one and/or multiple targets from the hidden patterns. In another embodiment, the deep machine-learning engine can identify antibodies/nanobodies having cross-reactivity to one and/or multiple targets from the hidden patterns. In other embodiments, the deep machine-learning engine can map the epitopes of antibodies/nanobodies and/or find out where antibodies/nanobodies bind to their antigens. The deep-learning engine can expand the library of antibody/nanobody lead candidates for one and/or multiple targets. The deep-learning engine can repurpose the antibody/nanobody sequences of one target for another target by finding the hidden patterns across antibody/nanobody sequence libraries.

In an aspect, the present disclosure provides a computer-implemented method for generating a set of candidate variant amino acid sequences of an antibody, a nanobody, or a fragment thereof, having binding ability to a target protein, comprising: (a) obtaining a set of seed amino acid sequences; and (b) processing the set of seed amino acid sequences using a first trained machine learning algorithm to generate the set of candidate variant amino acid sequences, wherein the first trained machine learning algorithm is trained with first training data comprising a set of training amino acid sequences for the target protein, wherein the first trained machine learning algorithm is further trained through a transfer learning method using a second trained machine learning algorithm, wherein the second trained machine learning algorithm is trained with second training data comprising a set of training amino acid sequences for a second target protein, wherein the second target protein is different from the target protein.

In some embodiments, the set of seed amino acid sequences comprises antibody variable regions (Fvs). In some embodiments, the Fvs comprise complementarity determining regions (CDRs) or frameworks (FWRs). In some embodiments, the Fvs comprise CDRs. In some embodiments, the CDRs comprise CDR1, CDR2, or CDR3. In some embodiments, the CDRs comprise CDR3. In some embodiments, the Fvs comprise heavy chains (VH) or light chains (VL). In some embodiments, the Fvs comprise frameworks (FWRs). In some embodiments, the FWRs comprise FWR1, FWR2, FWR3, or FWR4. The method of claim 1, wherein the target protein comprises at least a portion of a target antigen. In some embodiments, the target antigen comprises at least a portion of SARS-CoV-2 virus.

In some embodiments, the second target protein is selected from the group consisting of Ranibizumab, Trastuzumab, chicken ovalbumin, and yeast display scFV. In some embodiments, the second target protein comprises at least two of Ranibizumab, Trastuzumab, chicken ovalbumin, and yeast display scFV. In some embodiments, the second target protein comprises at least three of Ranibizumab, Trastuzumab, chicken ovalbumin, and yeast display scFV. In some embodiments, the second target protein comprises Ranibizumab, Trastuzumab, chicken ovalbumin, and yeast display scFV.

In some embodiments, the set of training amino acid sequences for the second target protein has a larger number of amino acid sequences than the set of training amino acid sequences for the target protein.

In some embodiments, the method further comprises, prior to (b), pre-processing the set of seed amino acid sequences to have a same sequence length. In some embodiments, the method further comprises, prior to (b), pre-processing the set of seed amino acid sequences at least in part by generating a numerical representation of the set of seed amino acid sequences. In some embodiments, the numerical representation comprises a matrix. In some embodiments, the matrix comprises a one-hot encoding of the set of amino acid sequences. In some embodiments, the matrix comprises a one-hot encoding of amino acid bio-physicochemical values. In some embodiments, the amino acid bio-physicochemical values are selected from isoelectric point, volume, hydrophobicity, solubility, charge, and solvent-accessible surface area (SASA). In some embodiments, the method further comprises, prior to (b), performing an embedding pre-processing on the set of seed amino acid sequences. In some embodiments, the matrix comprises a one-hot encoding of amino acid bio-physicochemical values that improves a classification models (e.g., Decision Tree (DT), Support Vector Machines (SVM), Random Forests (RF), and k-nearest neighbors (KNN)).

In some embodiments, the first trained machine learning algorithm or the second trained machine learning algorithm comprises a deep learning model. In some embodiments, the deep learning model comprises a deep generative model. In some embodiments, the deep generative model comprises at least one of an Autoencoder (AE), a Variational Autoencoder (VAE) and a Residual Neural Network (Resnet), a Generative Adversarial Network (GAN) and a Reinforcement Learning (RL), and a Graph Neural Network (GNN). In some embodiments, the deep generative model comprises at least two of the GAN, the AE, the VAE, the Resnet, the RL and the GNN. In some embodiments, the deep generative model comprises at least three of the GAN, the AE, the VAE, the Resnet, the RL, and the GNN. In some embodiments, the deep generative model comprises the GAN, the VAE, the Resnet, the RL, and the GNN.

In some embodiments, the first trained machine learning algorithm or the second trained machine learning algorithm comprises an encoder and a decoder. In some embodiments, the encoder comprises at least one of residual layers, convolutional layers, and dense layers. In some embodiments, the encoder comprises at least two of residual layers, convolutional layers, and dense layers. In some embodiments, the encoder comprises residual layers, convolutional layers, and dense layers.

In some embodiments, the method further comprises performing, using the encoder, a dimensionality reduction of the set of seed amino acid sequences. In some embodiments, the dimensionality reduction comprises generating a two-dimensional feature space. In some embodiments, the method further comprises improving the clustering features of the set of seed amino acid sequences based on sequence similarity or sequence diversity. In some embodiments, the clustering comprises using K-means or Gaussian mixture model (GMM).

In some embodiments, the method further comprises repurposing of the set of seed amino acid sequences. In some embodiments, the repurposing comprises varying the set of seed amino acid sequences that has binding to one target to have binding to an additional target.

In some embodiments, the dimensionality reduction comprises principal component analysis (PCA), t-distributed Stochastic Neighbor Embedding (t-SNE), uniform manifold approximation and projection (UMAP), variational autoencoder (VAE), or single-cell Consensus Clusters of Encoded Subspaces (scCCESS). In some embodiments, the method further comprises reconstructing, using the decoder, an input sequence from the two-dimensional feature space. In some embodiments, the decoder comprises at least one of convolutional layers and convolutional transpose layers. In some embodiments, the decoder comprises convolutional layers and convolutional transpose layers.

In some embodiments, the first trained machine learning algorithm and the second trained machine learning algorithm are trained without use of structural data.

In some embodiments, the method further comprises processing the set of candidate variant amino acid sequences to determine a therapeutic property selected from single specificity, multiple specificity, single cross-reactivity, and multiple cross-reactivity.

In some embodiments, the method further comprises processing the set of candidate variant amino acid sequences to determine molecular characteristics of the set of candidate variant amino acid sequences. In some embodiments, the molecular characteristics comprise at least one of amino acid distribution, amino acid frequency, amino acid length distribution, sequence similarity, sequence diversity, total charge distribution, hydrophobicity, net charge, stability, solubility, isoelectric point, solvent accessible surface area (SASA), binding affinity, epitope-paratrope interaction, epitope prediction, humanization, humaneness, developability, manufacturability, half-life, pharmacokinetic, yield, aggregation, function, clearance, viscosity, immunogenicity, single-target specificity, multi-target specificity, single-target cross-reactivity, and multi-target cross-reactivity. In some embodiments, the method further comprises determining sequence similarity between the set of candidate variant amino acid sequences and the set of seed amino acid sequences. In some embodiments, the method further comprises determining the sequence similarity using at least one of a Bilingual Evaluation Understudy (BLEU) method, a Jensen-Shannon divergence (JSD) method, and a Needleman-Wunch (NW) method. In some embodiments, the method further comprises determining sequence diversity between the set of candidate variant amino acid sequences and the set of seed amino acid sequences. In some embodiments, the method further comprises determining the sequence diversity at least in part by measuring a number of shared n-grams for one or more values of n. In some embodiments, the shared n-grams comprise 2-grams, 3-grams, and 4-grams.

In some embodiments, the method further comprises determining specificity or binding affinity of the set of candidate variant amino acid sequences to the target protein.

In some embodiments, the method further comprises filtering out at least one of the set of candidate variant amino acid sequences having undesired molecular characteristics. In some embodiments, the method further comprises selecting or ranking at least one of the set of candidate variant amino acid sequences based at least in part on having desired molecular characteristics. In some embodiments, the method further comprises validating at least one of the selected or ranked candidate variant amino acid sequences.

In some embodiments, the set of candidate variant amino acid sequences correspond to an antibody, a nanobody, or a fragment thereof. In some embodiments, the antibody is a monoclonal antibody, a bispecific antibody, or a trispecific antibody.

In some embodiments, the method further comprises determining a three-dimensional structure of the antibody, the nanobody, or the fragment thereof, based at least in part on the set of candidate variant amino acid sequences.

In some embodiments, the method further comprises predicting a humanized antibody, nanobody, or fragment variable region based at least in part on the set of candidate variant amino acid sequences.

In some embodiments, the method further comprises performing a deep learning-based classification to group the set of candidate variant amino acid sequences having binding or specificity to a same target. In some embodiments, the deep learning-based classification comprises a deep neural network (DNN). In some embodiments, the method further comprises performing automated and multimodal screening of the set of candidate variant amino acid sequences. In some embodiments, the automated and multimodal screening comprises using an ensemble model to identify a shortlist of antibodies, nanobodies, or fragments thereof with desirable therapeutics properties.

In another aspect, the present disclosure provides a computer-implemented method for generating a set of candidate variant amino acid sequences mutations of an antibody, a nanobody, or a fragment thereof, having binding ability to a target protein, comprising: (a) obtaining a set of seed amino acid sequences; and (b) processing the set of seed amino acid sequences using a trained machine learning algorithm to generate the set of candidate variant amino acid sequences, wherein the trained machine learning algorithm comprises at least one of a generative adversarial network (GAN) and Reinforcement Learning (RL).

In some embodiments, the trained machine learning algorithm is trained with training data comprising a set of training amino acid sequences for the target protein. In some embodiments, the set of training amino acid sequences comprises antibody variable regions (Fvs). In some embodiments, the Fvs comprise complementarity determining regions (CDRs) or frameworks (FWRs). In some embodiments, the Fvs comprise CDRs. In some embodiments, the CDRs comprise CDR1, CDR2, or CDR3. In some embodiments, the CDRs comprise CDR3. In some embodiments, the Fvs comprise heavy chains (VH) or light chains (VL). In some embodiments, the Fvs comprise frameworks (FWRs). In some embodiments, the FRs comprise FWR1, FWR2, FWR3, or FWR4. In some embodiments, the target protein comprises at least a portion of a target antigen. In some embodiments, the target antigen comprises at least a portion of SARS-CoV-2 virus.

In some embodiments, the second target protein is selected from the group consisting of Ranibizumab, Trastuzumab, chicken ovalbumin, and yeast display scFV. In some embodiments, the second target protein comprises at least two of Ranibizumab, Trastuzumab, chicken ovalbumin, and yeast display scFV. In some embodiments, the second target protein comprises at least three of Ranibizumab, Trastuzumab, chicken ovalbumin, and yeast display scFV. In some embodiments, the second target protein comprises Ranibizumab, Trastuzumab, chicken ovalbumin, and yeast display scFV.

In some embodiments, the method further comprises, prior to (b), pre-processing the set of seed amino acid sequences to have a same sequence length. In some embodiments, the method further comprises, prior to (b), pre-processing the set of seed amino acid sequences at least in part by generating a numerical representation of the set of seed amino acid sequences. In some embodiments, the numerical representation comprises a matrix. In some embodiments, the matrix comprises a one-hot encoding of the set of amino acid sequences. In some embodiments, the matrix comprises a one-hot encoding of amino acid bio-physicochemical values. In some embodiments, the amino acid bio-physicochemical values are selected from isoelectric point, volume, hydrophobicity, solubility, charge, and solvent-accessible surface area (SASA). In some embodiments, the method further comprises, prior to (b), performing an embedding pre-processing on the set of seed amino acid sequences.

In some embodiments, the trained machine learning algorithm comprises the GAN and the RL. In some embodiments, the trained machine learning algorithm is trained without use of structural data.

In some embodiments, the method further comprises processing the set of candidate variant amino acid sequences to determine a therapeutic property selected from single specificity, multiple specificity, single cross-reactivity, and multiple cross-reactivity.

In some embodiments, the method further comprises processing the set of candidate variant amino acid sequences to determine molecular characteristics of the set of candidate variant amino acid sequences. In some embodiments, the molecular characteristics comprise at least one of amino acid distribution, amino acid frequency, amino acid length distribution, sequence similarity, sequence diversity, total charge distribution, hydrophobicity, net charge, stability, solubility, isoelectric point, solvent accessible surface area (SASA), binding affinity, epitope-paratrope interaction, epitope prediction, humanization, humaneness, developability, manufacturability, half-life, pharmacokinetic, yield, aggregation, function, clearance, viscosity, immunogenicity, single-target specificity, multi-target specificity, single-target cross-reactivity, and multi-target cross-reactivity. In some embodiments, the method further comprises determining sequence similarity between the set of candidate variant amino acid sequences and the set of seed amino acid sequences. In some embodiments, the method further comprises determining the sequence similarity using at least one of a Bilingual Evaluation Understudy (BLEU) method, a Jensen-Shannon divergence (JSD) method, and a Needleman-Wunch (NW) method. In some embodiments, the method further comprises determining sequence diversity between the set of candidate variant amino acid sequences and the set of seed amino acid sequences. In some embodiments, the method further comprises determining the sequence diversity at least in part by measuring a number of shared n-grams for one or more values of n. In some embodiments, the shared n-grams comprise 2-grams, 3-grams, and 4-grams.

In some embodiments, the method further comprises determining specificity or binding affinity of the set of candidate variant amino acid sequences to the target protein.

In some embodiments, the method further comprises filtering out at least one of the set of candidate variant amino acid sequences having undesired molecular characteristics. In some embodiments, the method further comprises selecting or ranking at least one of the set of candidate variant amino acid sequences based at least in part on having desired molecular characteristics. In some embodiments, the method further comprises validating at least one of the selected or ranked candidate variant amino acid sequences.

In some embodiments, the set of candidate variant amino acid sequences correspond to an antibody, a nanobody, or a fragment thereof. In some embodiments, the antibody is a monoclonal antibody, a bispecific antibody, or a trispecific antibody.

In some embodiments, the method further comprises predicting an epitope based at least in part on the set of candidate variant amino acid sequences.

In some embodiments, the method further comprises determining a three-dimensional structure of the antibody, the nanobody, or the fragment thereof, based at least in part on the set of candidate variant amino acid sequences.

In some embodiments, the method further comprises predicting a humanized antibody variable region based at least in part on the set of candidate variant amino acid sequences.

In some embodiments, the method further comprises performing a deep learning-based classification to group the set of candidate variant amino acid sequences having binding or specificity to a same target. In some embodiments, the deep learning-based classification comprises a deep neural network (DNN). In some embodiments, the method further comprises performing automated and multimodal screening of the set of candidate variant amino acid sequences. In some embodiments, the automated and multimodal screening comprises using an ensemble model to identify a shortlist of antibodies, nanobodies, or fragments thereof with desirable therapeutics properties.

In another aspect, the present disclosure provides computer-implemented method for predicting an amino acid sequence or structure of an antibody paratrope or fragment thereof, having binding ability to a target protein, comprising: (a) obtaining a structure and amino acid sequences of an epitope of the target protein; and (b) processing the structure and amino acid sequences of the epitope of the target protein using a trained machine learning algorithm to predict the amino acid sequence or structure of the antibody paratrope or fragment thereof.

In some embodiments, the target protein comprises at least a portion of a target antigen. In some embodiments, the target antigen comprises at least a portion of SARS-CoV-2 virus.

In some embodiments, the trained machine learning algorithm is trained with training data comprising a set of training amino acid sequences or structures for the target protein. In some embodiments, the trained machine learning algorithm comprises a deep learning model. In some embodiments, the deep learning model comprises a deep generative model. In some embodiments, the deep generative model comprises at least one of a generative adversarial network (GAN), an autoencoder (AE), a variational autoencoder (VAE), a residual neural network (Resnet), Reinforcement Learning (RL), and a Graph Neural Network (GNN). In some embodiments, the deep generative model comprises at least two of the GAN, the AE, the VAE, the Resnet, the RL, and the GNN. In some embodiments, the deep generative model comprises at least three of the GAN, the VAE, the Resnet, the RL, and the GNN. In some embodiments, the deep generative model comprises at least four of the GAN, the VAE, the Resnet, the RL, and the GNN. In some embodiments, the deep generative model comprises the GAN, the VAE, the Resnet, the RL, and the GNN.

In some embodiments, (a) comprises obtaining a 3-dimensional structure and sequences of the target antigen, and wherein (b) comprises predicting the 3-dimensional structure of the antibody paratrope of fragment thereof.

In some embodiments, the method further comprises processing the predicted sequence or structure of the antibody paratrope of fragment thereof to determine molecular characteristics of the antibody paratrope of fragment thereof. In some embodiments, the molecular characteristics comprise at least one of amino acid distribution, amino acid frequency, amino acid length distribution, sequence similarity, sequence diversity, total charge distribution, hydrophobicity, net charge, stability, solubility, isoelectric point, solvent accessible surface area (SASA), binding affinity, epitope-paratrope interaction, epitope prediction, humanization, humaneness, developability, manufacturability, half-life, pharmacokinetic, yield, aggregation, function, clearance, viscosity, immunogenicity, single-target specificity, multi-target specificity, single-target cross-reactivity, and multi-target cross-reactivity.

In some embodiments, the method further comprises processing determining specificity or binding affinity of the antibody paratrope of fragment thereof to the target antigen.

In some embodiments, the method further comprises processing validating the predicted sequence or structure of the antibody paratrope or fragment thereof.

In some embodiments, (b) comprises predicting the sequence and the structure of the antibody paratrope or fragment thereof.

In some embodiments, the method further comprises using a deep learning-based model to analyze antigen epitope-antibody paratope interactions. In some embodiments, the deep learning-based model uses only amino acid sequences to predict epitopes, and wherein the method further comprises identifying antibodies, nanobodies, and fragments thereof with a desired mechanism of action.

In some embodiments, the method comprises quantitative metrics or qualitative metrics. In some embodiments, the quantitative metrics or qualitative metrics comprise at least one of accuracy, precision, perplexity (PPL), Area under the ROC Curve (AUC), amino acid recovery (AAR), and contact amino acid recovery (CAAR). In some embodiments, the method further comprises measuring at least one of contact accuracy, interface RMSD, and ligand RMSD.

In some embodiments, the method further comprises analyzing physicochemical attributes of the predicted antibody paratope or fragment thereof. In some embodiments, the physicochemical attributes comprise at least one of hydrophobicity, polarity, exposed surface, and accessibility.

In some embodiments, the method further comprises using a deep learning-based automated and multimodal screening method to identify the desirable paratope or fragment thereof faster, more accurately, and more efficiently. In some embodiments, the method further comprises classifying antibodies, nanobodies, or fragments thereof, based on their binding capabilities. In some embodiments, the classifying comprises use of a deep neural network (DNN).

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 illustrates an overview representation of an example system that identifies candidate amino acid sequences using a variational autoencoder (VAE) Resnet transfer learning (VAEResTL) deep generative model trained on initial amino acid sequence(s).

FIG. 2A is a graph demonstrating the total loss during training for the VAEResTL model.

FIG. 2B is a graph demonstrating the total loss during training for the VAERes model.

FIG. 3A provides a Visualize Comparison of molecular characteristics, amino acid distribution, between Seed (SARS-CoV-2 seed sequences, Yellow), VAERes (generated sequences by VAERes for SARS-CoV-2, Violet) and VAEResTL (generated sequences by VAEResTL for SARS-CoV-2, Turquoise).

FIG. 3B provides a Visualize Comparison of molecular characteristics, amino acid length distribution, between Seed (SARS-CoV-2 seed sequences, Yellow), VAERes (generated sequences by VAERes for SARS-CoV-2, Violet) and VAEResTL (generated sequences by VAEResTL for SARS-CoV-2, Turquoise).

FIG. 3C provides a Visualize Comparison of molecular characteristics, total charge distribution, between Seed (SARS-CoV-2 seed sequences, Yellow),VAERes (generated sequences by VAERes for SARS-CoV-2, Violet) and VAEResTL (generated sequences by VAEResTL for SARS-CoV-2, Turquoise).

FIG. 3D provides a Visualize Comparison of molecular characteristics, Eisenberg hydrophobicity, between Seed (SARS-CoV-2 seed sequences, Yellow),VAERes (generated sequences by VAERes for SARS-CoV-2, Violet) and VAEResTL (generated sequences by VAEResTL for SARS-CoV-2, Turquoise).

FIG. 3E provides a Visualize Comparison of molecular characteristics, Eisenberg Hydrophobic moment, between Seed (SARS-CoV-2 seed sequences, Yellow),VAERes (generated sequences by VAERes for SARS-CoV-2, Violet) and VAEResTL (generated sequences by VAEResTL for SARS-CoV-2, Turquoise).

FIG. 4A1 is a Machine Learning (ML) Visualization that demonstrates a position proposed map. Heatmap visualization showing the count of observed seed sequences for SARS-CoV-2.

FIG. 4B1 is a Machine Learning (ML) Visualization that demonstrates a position proposed map. Heatmap visualization showing the count of VAERes-generated sequences or SARS-CoV-2 from (left) and to (right) each amino acid at each sequence position.

FIG. 4C1 is a Machine Learning (ML) Visualization that demonstrates a position proposed map. Heatmap visualization showing the count of VAEResTL-generated sequences for SARS-CoV-2 from (left) and to (right) each amino acid at each sequence position (seed and VAERes-generated and VAEResTL-generated sequences having length 20).

FIG. 4A2 is a Machine Learning (ML) Visualization that demonstrates sequence logos for Seeds SARS-CoV-2.

FIG. 4B2 is a Machine Learning (ML) Visualization that demonstrates the VAERes-generated sequences for SARS-CoV-2.

FIG. 4C2 is a Machine Learning (ML) Visualization that demonstrates the VAEResTL-generated sequences for SARS-CoV-2 are based on residue frequency.

FIG. 5A illustrates In-Silico Screening of predicted CDR-H3 sequences for further assessment, Net charge of CDR-H3 sequence.

FIG. 5B illustrates In-Silico Screening of predicted CDR-H3 sequences for further assessment, CDR-H3 hydrophobicity.

FIG. 5C illustrates In-Silico Screening of predicted CDR-H3 sequences for further assessment, CamSol solubility score.

FIG. 5D illustrates In-Silico Screening of predicted CDR-H3 sequences for further assessment, CDR-H3 stability score.

FIG. 5E illustrates In-Silico Screening of predicted CDR-H3 sequences for further assessment, The minimum NetMHCIIpan % Rank (<2 strong binder; <10 weak binder) across all possible 15-mers for a given CDR-H3 sequences and across all 26 HLA alleles.

FIG. 5F illustrates In-Silico Screening of predicted CDR-H3 sequences for further assessment, The average NetMHCIIpan % Rank across all possible 15-mers for a given CDR-H3 sequences and across all 26 HLA alleles.

FIG. 6 illustrates an overview representation of an example system that identifies amino acid sequences based on their single or multiple target specificity and/or antibody amino acid sequences' cross-reactivity to one or multiple targets using a variational autoencoder (VAE) Resnet dimensionality reduction (VAEResDR) model, which is a deep learning-based feature extractor model trained on amino acid sequence(s).

FIG. 7A is a visualization plot of dimensionality reduction (DR) for the baseline PCA. CDR-H3 sequences for three target antigens in 5 clusters were processed.

FIG. 7B is a visualization plot of dimensionality reduction for the baseline t-SNE. CDR-H3 sequences for three target antigens in 5 clusters were processed.

FIG. 7C is a visualization plot of dimensionality reduction for the baseline UMAP. CDR-H3 sequences for three target antigens in 5 clusters were processed.

FIG. 7D is a visualization plot of dimensionality reduction for the baseline VAE. CDR-H3 sequences for three target antigens in 5 clusters were processed.

FIG. 7E is a visualization plot of dimensionality reduction for the baseline scCCESS (AE). CDR-H3 sequences for three target antigens in 5 clusters were processed.

FIG. 7F is a visualization plot of dimensionality reduction for the baseline VAEDR. CDR-H3 sequences for three target antigens in 5 clusters were processed.

FIG. 7G is a visualization plot of dimensionality reduction for the VAEResDR. CDR-H3 sequences for three target antigens in 5 clusters were processed.

FIG. 7H is a visualization plot of dimensionality reduction for baseline PCA. CDR-H3 sequences for four target antigens in 6 clusters were processed.

FIG. 7I is a visualization plot of dimensionality reduction for baseline t-SNE. CDR-H3 sequences for four target antigens in 6 clusters were processed.

FIG. 7J is a visualization plot of dimensionality reduction for baseline uniform manifold approximation and projection (UMAP). CDR-H3 sequences for four target antigens in 6 clusters were processed.

FIG. 7K is a visualization plot of dimensionality reduction for baseline VAE. CDR-H3 sequences for four target antigens in 6 clusters were processed.

FIG. 7L is a visualization plot of dimensionality reduction for baseline scCCESS (AE). CDR-H3 sequences for four target antigens in 6 clusters were processed.

FIG. 7M is a visualization plot of dimensionality reduction for the baseline VAEDR. CDR-H3 sequences for four target antigens in 6 clusters were processed.

FIG. 7N is a visualization plot of dimensionality reduction for the VAEResDR. CDR-H3 sequences for four target antigens in 6 clusters were processed.

FIG. 8A is a bar chart showing Clustering Comparison to demonstrate the dimensionality reduction (DR) methods' performance on CDR-H3 sequences in 5 clusters.

FIG. 8B is a bar chart showing Clustering Comparison to demonstrate the DR methods' performance on CDR-H3 sequences in 6 clusters.

FIG. 9A illustrates a VAEResDR two-dimensional Hidden Patterns Analysis. 2-dimensional visualization by VAEResDR method for four targets, 6 clusters. CDR-H3 Sequences sharing the same latent space are marked as group (a) and CDR-H3 sequences in different latent spaces are marked as group (b) and (c).

FIG. 9B illustrates a VAEResDR two-dimensional Hidden Patterns Analysis, including the biophysical metric of hydrophobicity (H).

FIG. 9C illustrates a VAEResDR two-dimensional Hidden Patterns Analysis, including the biophysical metric of net-charge.

FIG. 9D illustrates a VAEResDR two-dimensional Hidden Patterns Analysis, including the biophysical metric of Iso-electric Point (ISO).

FIG. 9E illustrates a VAEResDR two-dimensional Hidden Patterns Analysis, including the machine learning (ML) visualization Heatmap plot for group (a).

FIG. 9F illustrates a VAEResDR two-dimensional Hidden Patterns Analysis, including the ML visualization Heatmap plot for group (b).

FIG. 9G illustrates a VAEResDR two-dimensional Hidden Patterns Analysis, including the ML visualization Heatmap plot for group (c).

FIG. 10A is an amino acid distribution plot comparing Seed sequences (SARS-CoV-2 seed sequences, orange), (OVA seed sequences, Turquoise), (Yeast display scFV seed sequences, Violet), (Rani seed sequences, Yellow), and includes a schematic of the comparison of the training seed amino acid sequences.

FIG. 10B is an amino acid length distribution plot comparing Seed sequences (SARS-CoV-2 seed sequences, orange), (OVA seed sequences, Turquoise), (Yeast display scFV seed sequences, Violet), (Rani seed sequences, Yellow), and includes a schematic of comparison of the training seed amino acid sequences, and a schematic of the comparison of the training seed amino acid sequences.

FIG. 10C is a total charge distribution plot comparing seed sequences (SARS-CoV-2 seed sequences, orange), (OVA seed sequences, Turquoise), (Yeast display scFV seed sequences, Violet), (Rani seed sequences, Yellow), and includes a schematic of comparison of the training seed amino acid sequences.

FIG. 10D is an Eisenberg hydrophobicity plot comparing seed sequences (SARS-CoV-2 seed sequences, orange), (OVA seed sequences, Turquoise), (Yeast display scFV seed sequences, Violet), (Rani seed sequences, Yellow), and includes a schematic of comparison of the training seed amino acid sequences, and a schematic of the comparison of the training seed amino acid sequences.

FIG. 10E1 is a heatmap visualization plot for SARS-CoV-2 (Seed) CDR-H3 sequences.

FIG. 10E2 is a logo visualization plot for SARS-CoV-2 (Seed) CDR-H3 sequences,

FIG. 10F1 is a heatmap visualization plot for OVA (Seed) CDR-H3 sequences.

FIG. 10F2 is a logo visualization plot for OVA (Seed) CDR-H3 sequences, a schematic on how the training sequences are distributed.

FIG. 10G1 is Heatmap visualization plot for Rani (Seed) CDR-H3 sequences.

FIG. 10G2 is a Logo visualization plot for Rani (Seed) CDR-H3 sequences, including a schematic on how the training sequences are distributed.

FIG. 10H1 is a Heatmap visualization plot for Yeast display scFV (Seed) CDR-H3 sequences.

FIG. 10H2 is a Logo visualization plot for Yeas (Seed) CDR-H3 sequences, including a schematic on how the training sequences are distributed.

FIG. 11 shows an overview of the AbGAN method.

FIG. 12A shows Biological Characteristics and Machine Learning Metrics for AbGAN Fv generated SARS-CoV-2, HIV, and OVA sequences compared to Seed Fv SARS-CoV-2, HIV, and OVA sequences.

FIG. 12B shows UMAP visualization plot illustrating the AbGAN Fv generated SARS-CoV-2, HIV, and OVA sequences compared to Seed Fv SARS-CoV-2, HIV, and OVA sequences in a two-dimensional latent space.

FIGS. 13A-13B show Bar Chart Generative Models Performance Comparison, including: (FIG. 13A) Biological Characteristics Metrics for AbGAN, LSTM, VAEGP, and DCGAN Fv generated SARS-CoV-2, HIV, and OVA sequences compared to Seed Fv SARS-CoV-2, HIV, and OVA sequences; and (FIG. 13B).

FIG. 14A-14D show Visualize Comparison of molecular characteristics between Seed (SARS-CoV-2 seed sequences, Orang), AbGAN (AbGAN-generated sequences for SARS-CoV-2, Torque) and LSTM (LSTM-generated sequences for SARS-CoV-2, Purple), VAEGP (VAEGP-generated sequences for SARS-CoV-2, Pink), and DCGAN (DCGAN-generated sequences for SARS-CoV-2, Yellow). Horizontal dashed lines account for the mean. Whiskers extend to the most extreme non-outlier data points. The figures include (FIG. 14A) total charge distribution, (FIG. 14B) amino acid distribution, (FIG. 14C) amino acid length distribution, and (FIG. 14D) Eisenberg hydrophobicity.

FIG. 15A-15D show In-Silico Screening of predicted Fv sequences. Histograms present the parameter distributions of AbGAN-generated and seed SARS-CoV-2 sequences for different filtering steps. Boxes show the filtering cut-off for the therapeutic index in a clinical setting. The figures include (FIG. 15A) Fv sequences net-charge, (FIG. 15B) Fv sequences H, (FIG. 15C) Fv sequences stability score, and (FIG. 15D) CamSol solubility score.

FIG. 16 shows an overview of the method in antibody classification.

FIG. 17A-17C show receiver-operating characteristic (ROC) Plots. The ROC curves with positive samples threshold at 1.0. Four classification methods of decision tree (DT), K-nearest-neighbor (KNN), support vector machine (SVM), and random forest (RF) were tested on: (FIG. 17A) 2 classes of Tras-Positive and Tras-Negative; (FIG. 17B) 4 classes of Tras-Positive, Tras-Negative, Rani-Positive, and Rani-Negative; and (FIG. 17C) 5 classes of OVA, Tras-Positive, Tras-Negative, Rani-Positive, and Rani-Negative.

FIG. 18 illustrates an example of antibody paratope matching with antigen epitope. No match between epitope-paratope residues results in no affinity to antigen. Poor matches between epitope-paratope residues result in low affinity to antigen, and high-quality matches between epitope-paratope residue results in a high affinity to antigen.

FIG. 19 illustrates an example of an overview of a Deep Learning Model that is developed and trained by antigen paratope and 3D structure to design and predict antibody paratope and 3D structure by accurately modeling the epitope-paratope interactions.

FIG. 20 illustrates that the VAEResTL, a deep generative model, is an end-to-end trainable model which comprises a convolutional Variational Autoencoder (VAE) that adopts a Resnet structure and is enhanced with a Transfer Learning (TL) technique. The method utilizes only the amino acid sequence data without the need for structural data and designs a large library of antibody mutations.

FIG. 21 shows a Visualize Comparison of molecular characteristics between Seed-sequences (Yellow), VAERes-generated sequences (Violet) and VAEResTL-generated sequences (Turquoise). Horizontal dashed lines account for the mean. Whiskers extend to the most extreme non-outlier data points. (A) amino acid distribution, (B) amino acid length distribution, (C) total charge distribution, (D) Eisenberg hydrophobicity.

FIG. 22 shows a visualization of machine learning (ML). Heatmap visualization showing: (A1) the count of observed seed sequences. (B1) the count of VAERes-proposed sequences from (left) and to (right) each amino acid at each sequence position. (C1) the count of VAEResTL-proposed sequences from (left) and to (right) each amino acid at each sequence position. (Seed and VAERes-generated and VAEResTL-generated sequences have the length of 20). Sequence logo visualizations. (A2) Sequence logos for Seeds, (B2) VAERes-generated sequences (C2) VAEResTL-generated sequences fare based on residue frequency.

FIG. 23 illustrates an example of the model for the dimensionality reduction of antibody CDR-H3 sequences. A Resnet-adopted convolutional VAE was designed, referred to as VAEResDR. The VAEResDR comprises an encoder and a decoder. The encoder scales down high-dimensional input sequences into a two-dimensional feature space, while the decoder reconstructs the sequences from these two-dimensional features.

FIG. 24 shows a Visualization: 2-dimensional representation plots. Panel A to G representing PCA, t-SNE, UMPA, VAE, scCCESS (AE), VAEDR and VAEResDR for three targets, 5 clusters consecutively. Figure H to N representing PCA, t-SNE, UMPA, VAE, scCCESS (AE), VAEDR and VAEResDR for four targets, 6 clusters consecutively. OVA (orange dots), Yeast (red dots), Tars-Positive (yellow dots), Tras-Negative (turquoise dots), Rani-Positive (blue dots), and Rani-negative (green dots)).

FIG. 25 shows a bar chart clustering comparison to demonstrate the DR methods' performance. The best performance is ranked 1 and the worst performance is ranked 7. The color codes for rank 1, 2, 3, 4, 5, 6, and 7 are blue, orange, green, red, purple, brown, and pink consecutively.

FIG. 26 shows a Hidden Patterns Analysis: Biophysical and ML visualization Heatmap analysis. (A) 2-dimensional visualization by VAEResDR method for four targets, 6 clusters. CDR-H3 Sequences sharing the same latent space are marked as group (a) and CDR-H3 sequences in different latent spaces are marked as group (b) and (c). (B) biophysical metrics of hydrophobicity (H) (C) net-charge, and (D) ISO. (E), (F), (G), and (H) are heatmap plots for group (a), (b), and (c) sequentially.

FIG. 27 shows a Visualize Comparison of molecular characteristics between Seed-sequences (Yellow), VAERes-generated sequences (Violet) and VAEResTL-generated sequences (Turquoise). Horizontal dashed lines account for the mean. Whiskers extend to the most extreme non-outlier data points. (A) amino acid distribution, (B) amino acid length distribution, (C) total charge distribution, (D) Eisenberg hydrophobicity.

FIG. 28 illustrates an example of a Deep Learning Model that is developed and trained by antibody input data for Epitope Mapping. After the Deep Learning Model training and testing, a Ranking Model is used to analyze the antibody interactions with a given epitope to identify the best matches.

FIG. 29 illustrates an example illustration of a Deep Generative Engine. The VAEResTL, a deep generative model, is an end-to-end trainable model which comprises a convolutional Variational Autoencoder (VAE) that adopts a Resnet structure and is enhanced with a Transfer Learning (TL) technique. The method utilizes only the amino acid sequence data without the need for structural data and designs a large library of antibody mutations.

FIG. 30 shows a Visualize Comparison of molecular characteristics between Seed (SARS-CoV-2 seed sequences, Yellow), VAERes (VAERes-generated sequences for SARS-CoV-2, Violet), and VAEResTL (VAEResTL-generated sequences for SARS-CoV-2, Turquoise). Horizontal dashed lines account for the mean. Whiskers extend to the most extreme non-outlier data points. (A) amino acid distribution, (B) amino acid length distribution, (C) total charge distribution, and (D) Eisenberg hydrophobicity.

FIG. 31 shows a visualization of machine learning (ML). Heatmap visualization showing: (A1) the count of observed seed sequences. (B1) the count of VAERes-proposed sequences from (left) and to (right) each amino acid at each sequence position. (C1) the count of VAEResTL-proposed sequences from (left) and to (right) each amino acid at each sequence position. (Seed and VAERes-generated and VAEResTL-generated sequences have the length of 20). Sequence logo visualizations. (A2) Sequence logos for Seeds, (B2) VAERes-generated sequences (C2) VAEResTL-generated sequences fare based on residue frequency.

FIG. 32 shows an In-Silico Screening of predicted CDR-H3 sequences. Histograms present the parameter distributions of generated and seed SARS-CoV-2 sequences for different filtering steps. Boxes show the filtering cut-off for the therapeutic index in a clinical setting. (A) CDR-H3 net-charge. (B) CDR-H3 H. (C) CDR-H3 stability score. (D) CamSol solubility score. (E) the minimum NetMHCIIpan % Rank across all possible 15-mers for a given sequence and across all 26 HLA alleles. (F) the average NetMHCIIpan % Rank across all possible 15-mers for a given sequence and across all 26 HLA alleles.

FIG. 33 shows a model for the dimensionality reduction of antibody CDR-H3 sequences. The VAEResDR is a Resnet-adopted convolutional VAE, which comprises an encoder and a decoder. The encoder scales down high-dimensional input sequences into a two-dimensional feature space, while the decoder reconstructs the sequences from these two-dimensional features.

FIG. 34 shows a Visualization of 2-dimensional representation plots, with panels A to G representing PCA, t-SNE, UMPA, VAE, scCCESS (AE), VAEDR, and VAEResDR for three targets, 5 clusters consecutively; and panels H to N representing PCA, t-SNE, UMPA, VAE, scCCESS (AE), VAEDR, and VAEResDR for four targets, 6 clusters consecutively. OVA (orange dots), Yeast display scFV (red dots), Tars-Positive (yellow dots), Tras-Negative (turquoise dots), Rani-Positive (blue dots), and Rani-negative (green dots)).

FIG. 35 shows a Hidden Patterns Analysis, including Biophysical and ML visualization Heatmap analysis. (A) 2-dimensional visualization by VAEResDR method for four targets, 6 clusters. CDR-H3 Sequences sharing the same latent space are marked as group (a) and CDR-H3 sequences in different latent spaces are marked as group (b) and (c). (B) biophysical metrics of hydrophobicity (H) (C) net-charge, and (D) ISO. (E), (F), and (G) are heatmap plots for group (a), (b), and (c) sequentially.

FIG. 36 shows a computer system 3601 that is programmed or otherwise configured to perform analyses of the methods.

FIG. 37 shows an example method for generating a set of candidate amino acid sequences of an antibody, a nanobody, or a fragment thereof, having binding ability to a target antigen, using a transfer learning method.

FIG. 38 shows an example method for generating a set of candidate amino acid sequences of an antibody, a nanobody, or a fragment thereof, having binding ability to a target antigen, using a generative adversarial network (GAN).

FIG. 39 shows an example method for predicting a sequence or structure of an antibody paratrope or fragment thereof, having binding ability to a target antigen.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a nucleic acid” includes a plurality of nucleic acids, including mixtures thereof.

Antibodies play an important role in therapeutics discovery and vaccine manufacturing due to their ability to bind to one and/or more target antigens. The discovery and generation of antibodies, nanobodies or fragments thereof may be performed via in-silico methods.

The binding site of an antibody/nanobody may include a region referred to as the complementarity-determining region (CDR). Amongst CDRs, the third complementarity-determining region (CDR) of the heavy chain (CDR-H3) of an antibody/nanobody and/or fragment may be the region of highest sequence diversity, which may play a significant role in antigen recognition and dictates binding specificity and affinity. A deep neural network capable of learning from complex and highly variant sequences may be developed to model CDR-H3 sequences and/or to design CDR-H3 sequences mutations. However, increasing the depth of the neural network by adding more layers may lead to challenges such as overfitting and vanishing gradient problems. Moreover, existing deep learning approaches may need to be trained with large training datasets, and may be incapable of discovering and generating new antibodies, nanobodies or fragments thereof, when facing data scarcity for novel targets. In addition, there are a number of key therapeutics and developability properties to screen for identifying the lead antibody, nanobody, or fragment thereof candidates. The key therapeutics and developability properties may include but are not limited to antibody specificity to one or multiple targets, cross-reactivity to one or multiple targets, viscosity, clearance, stability, solubility, affinity, affinity maturation, epitope mapping, yield, aggregation, function, immunogenicity, humanization, humaneness, manufacturability, and developability. Therefore, there remains a need for tools that can rapidly, accurately, and efficiently analyze antibodies, nanobodies or fragments thereof, for their therapeutic and developability properties, in order to identify the lead candidates, perform analysis of the antibodies, nanobodies, or fragments thereof, for their key biophysical and therapeutic properties, such as specificity and cross-reactivity, and/or repurpose the antibodies, nanobodies, or fragments thereof, for different targets.

In addition, accurate prediction of antibody-antigen binding affinity requires time-consuming and expensive wet-lab experimentations. Therefore, a deep machine-learning method that can rapidly assess the antibody-antigen binding landscape is of significant importance.

Furthermore, besides the CDR-H3, the variable regions (Fv) in antibodies/nanobodies play a significant role in their specificity and binding affinity. However, existing artificial intelligence (AI) methods for antibody discovery mainly focus on antibodies' CDR-H3 region and not on the antibodies' variable fragment (Fv) regions. Thus, a deep machine-learning method that can predict antibody variable regions (Fvs), humanized Fvs, and antibody variable region mutations is of substantial importance.

Moreover, an essential task in antibody therapeutics discovery is to rapidly identify whether an antibody can recognize a specific epitope on an antigen surface and bind to it. Characterization of antibody-paratope:antigen-epitope interaction provides valuable mechanistic insights that can enhance antibody therapeutics' effectiveness. However, other methods may be overly costly and labor-intensive as repetitive wet-lab experimentations are required to sufficiently explore the antibody sequence space. Moreover, due to the extended time and the significant cost associated with those methods, such analysis may be used at the late stage of the discovery and development process and on a limited number of antibody candidates or antigenic variants. This reduces the number of goal shots among the antibody sequences galaxy and increases the risk of failure. Therefore, a deep machine-learning method that can enable an early stage, high-throughput, and efficient method for rapid and accurate antibody analysis, which relies on only amino acid sequences is of critical importance.

Moreover, the design of new therapeutic antibodies requires accurate paratope prediction for optimized antibody paratope-antigen epitope interactions that can be achieved through 3D structure modeling. In-silico antibody discovery for novel targets begins with antibody amino acid sequence data without antibody 3D structure data. The accurate modeling of antigen epitope-antibody paratope requires the model to incorporate the antigen epitope 3D structure to predict the antibody paratope 3D structure. Moreover, besides the backbone residue region in the antibody paratope, the surface side-chain conformation of the antibody paratope plays an important role in the binding interaction of the antibody paratope-antigen epitope. Therefore, the method may need to model both surface side chains and backbone residues in predicting antibody paratope 3D structure. However, the existing artificial intelligence (AI) methods developed for designing new antibody therapeutics do not incorporate the antigen 3D structure and do not model the surface side-chain conformation. Thus, there is a need for rapid and accurate antibody paratope design by the autoregressive generation of antibody paratope sequences while recursively improving antibody paratopes' backbones and side chain structures.

Described herein are systems and techniques for precisely designing and developing antibodies/nanobodies for existing and/or new targets. In some embodiments, the techniques may be used to design and develop antibodies, nanobodies, or fragments thereof against one target. In other embodiments, the techniques may be used to design and develop antibodies, nanobodies, or fragments thereof against multiple targets.

In some aspects, the present disclosure provides a deep learning-based antibody design tool. The antibody design tool may comprise a variational autoencoder (VAE) and residual neural network (Resnet), VAE-Resnet-based algorithm equipped with a transfer learning technique that generates antibody CDR3 sequences (e.g., particularly for novel and difficult targets for which the training data is often scarce), a generative adversarial network (GAN) and reinforcement learning (RL), GAN-RL-based algorithm that generates humanized antibody variable regions, and a conditioned deep learning graph neural network (GNN)-based 3D structure predictor that accurately designs antibodies while predicting antibody paratopes' 3D structures.

In some aspects, the present disclosure provides a deep learning-based antibody analysis tool. The antibody analysis may comprise a VAE-Resnet-based algorithm that reduces the dimensionality of antibodies by extracting the key features, analyzes antibodies for their key biological and key therapeutics (e.g., specificity and cross-reactivity), repurposes existing antibodies for other targets, and improves the performance of clustering models; a deep learning-based epitope mapping algorithm that predicts the epitopes using only amino acid sequences for early stage antibody assessment and for accurately identifying the antibody's mechanism of action; a deep learning-based classifier that accurately classifies the generated antibodies based on their binding ability to a given target; and BioOne-Hot preprocessing for improving the antibody classification.

A deep machine-learning engine is developed and trained using a variety of antibodies, nanobodies, or fragments thereof known to their targets. In some embodiments, the targets may include but are not limited to antigens. In other embodiments, the deep machine-learning engine may predict antibodies, nanobodies, or fragments sequences for new target antigens that were and/or were not among the antibodies, nanobodies and fragments sequences used for training. In other embodiments, a deep learning model may be used to perform epitope mapping/epitope prediction at the early stage of discovery using only amino acid sequences. In another embodiment, the deep machine-learning engine utilizes a given antigen epitope data to predict the 3D structure of the antibody, nanobody, or fragment paratopes that bind to the given antigen epitope. The deep machine-learning engine predicts the antibody, nanobody or fragment paratope 3D structure by modeling the antigen epitope-antibody paratope interactions. The predicted antibody/nanobody paratope 3D structure comprises the backbone residue region and the surface side-chains. In other embodiments, the deep machine-learning engine may predict the antibody paratope binding affinity by modeling the antigen epitope-antibody paratope interactions. In other embodiments, the deep machine-learning method may employ transfer-learning techniques to design antibodies, nanobodies, or fragments thereof for a novel target lacking sufficient training data. In some embodiments, the techniques may be used to design antibodies, nanobodies and/or antibodies/nanobodies mutations and/or to screen their biophysical properties. In another embodiment, the biophysical properties include but are not limited to net-charge, hydrophobicity, viscosity, clearance, solubility, stability, affinity, affinity maturation, isoelectric points, specificity, cross-reactivity, immunogenicity, humaneness, humanization, developability, manufacturability, half-life, pharmacokinetic, yield, aggregation, function, and antibody epitope mapping to identify the lead candidates for their therapeutics developability. The deep-learning engine can reduce dimensions in the antibodies, nanobodies, or fragments sequence data, and/or extracts the key biophysical features and/or group antibodies, nanobodies, or fragments sequence data into clusters and/or improve the clustering of antibodies, nanobodies, or fragments sequence and/or recognize hidden patterns across the clusters in their latent spaces and/or repurpose the antibodies, nanobodies, or fragments sequence data recognized from the hidden patterns from one target to be used against multiple targets. In some embodiments, the deep machine-learning engine can recognize antibodies, nanobodies and/or fragments or mutations thereof having specificity to one and/or multiple targets from the hidden patterns. In another embodiment, the deep machine-learning engine can identify antibodies, nanobodies and/or fragments or mutations thereof having cross-reactivity to one and/or multiple targets from the hidden patterns. In other embodiments, the deep machine-learning engine can map the epitopes of antibodies, nanobodies and/or find out where antibodies, nanobodies bind to their antigens. The deep-learning engine can expand the library of antibody, nanobody, or fragment lead candidates for one and/or multiple targets. The deep-learning engine can repurpose the antibody, nanobody, or fragment sequences of one target for another target by finding the hidden patterns across antibody, nanobody, or fragment sequence libraries.

According to some embodiments, high-capacity deep machine learning techniques for discovering amino acid sequences and amino acid sequence mutations for specific targets are provided. In some embodiments, amino acid sequences may include but are not limited to amino acid sequences for proteins such as antibodies, nanobodies and/or fragments or mutations thereof, and/or peptides, and/or peptidomimetics. In other embodiments, the deep machine-learning engine may require sufficient training data to predict amino acid sequences for one target. In another embodiment, the deep machine-learning engine may require sufficient training data to predict antibody, nanobody, or fragment sequences and amino acid sequence mutations for one and/or multiple targets. The training data may include but are not limited to deep sequencing data and/or amino acid sequences data for one and/or more antibodies, nanobodies and/or fragments or mutations thereof. In other embodiments, the deep machine-learning engine may be equipped with deep neural network models including but not limited to Residual Neural Network (Resnet) to predict antibodies, nanobodies, or fragments thereof for one and/or more targets. In another embodiment, the deep machine-learning engine is armed with a transfer learning technique to predict antibodies, nanobodies and/or fragments or mutations thereof for a new target facing data scarcity. In another embodiment, the deep machine-learning engine is equipped with a reinforcement learning to predict antibodies, nanobodies and/or fragments or mutations thereof for a new target facing data scarcity and one and/or more targets facing data scarcity. The proposed amino acid sequences may indicate amino acid sequences for each residue of proposed amino acid sequences that may include but are not limited to similar amino acid sequences and/or different amino acid sequences. The targets may include but are not limited to antigens. In another embodiment, the deep machine-learning engine reduces dimensions in the antibodies, nanobodies, or fragments sequence data and/or visualizes the antibodies/nanobodies sequence data in lower dimensions and/or groups antibodies, nanobodies, or fragments sequence data into clusters and/or improves the clustering performance and/or uncovers hidden patterns across the clusters in their latent spaces for antibody, nanobody, or fragment amino acid sequences analysis.

In some embodiments, the training data may include but are not limited to antibody, nanobody, or fragment amino acid sequences. The antibody, nanobody, or fragment amino acid sequences may include but are not limited to sequences of the full length of the heavy chains and the light chains of one and/or more antibodies and/or the full length of the heavy chains of one and/or more nanobodies. In other embodiments, the antibody, nanobody, or fragment amino acid sequences may include but are not limited to sequences of the variable regions (Fv). The variable regions may include but are not limited to antibody, nanobody, or fragment amino acid variable regions in the heavy chains (VH) and/or antibody amino acid variable regions in the light chains (VL). In another embodiment, the antibody, nanobody, or fragment amino acid sequences may include but are not limited to sequences of the Complementarity Determining Regions (CDR regions). The CDR regions are the residues of an antibody in both the heavy chains (CDR-H) and the light chains (CDR-L) and the residues of a nanobody in the heavy chains (CDR-H). In some embodiments, the antibody, nanobody, or fragment amino acid sequences are the sequences of CDRs in the heavy chains, including but not limited to CDR-H1, CDR-H2, and CDR-H3. In other embodiments, the antibody amino acid sequences are the sequences of CDRs in the light chains, including but not limited to CDR-L1, CDR-L2, and CDR-L3. In another embodiment, the amino acid sequences are the sequences of Frameworks (FWRs), including but not limited to FWRs in the heavy chains and light chains of an antibody and/or FWRs in the heavy chains of a nanobody. In some embodiments, the FWRs include FWR-H1, FWR-H2, FWR-H3, and FWR-H4 in the heavy chains and FWR-L1, FWR-L2, FWR-L3, and FWR-L4 in the light chains. In other embodiments, the antibody, nanobody, or fragment amino acid sequences are other fractions of sequences including but not limited to germlines of variable (V), diversity (D), joining (J), and junction in the heavy chains and variable (V), and joining (J) and junctions in the light chains.

In other embodiments, the training data may include but are not limited to antibodies, nanobodies, or fragment deep sequencing data. The deep sequencing data may be provided by Next Generation Sequencing (NGS) approaches of DNA and/or RNA of antibodies, nanobodies, or fragments for the full length of the heavy chains and/or the light chains, the variable regions in the heavy chain (VH) and/or the variable regions in the light chains (VL), CDRs and/or FWRs, and/or germlines of V, D, J and junctions of one and/or more antibodies/nanobodies. The NGS data may be obtained from laboratory experiments including but not limited to phage-display, hybridoma fusion, proteomics, NGS combined, single-cell screening and cloning, synthetic biology, and Crisper Cas9.

In other embodiments, the training data may include but are not limited to unlabeled and labeled antibodies, nanobodies, or fragment amino acid sequences and/or deep sequencing data for existing and/or new targets. The deep machine-learning engine may further receive amino acid sequences proposed by the deep machine-learning engine.

In some embodiments, the deep machine-learning engine comprises training the machine with antibody, nanobody, or fragment amino acid sequences, which are a series of distinct characteristics with a series of attributes. Each of the input distinct characteristics has attributes associated with its position within the initial and the end position of the distinct characteristics on the antibody, nanobody, or fragment amino acid sequences. In some embodiments, the input distinct characteristics are passed through a series of processing steps. In other embodiments, the method queries the input distinct characteristics that are within a defined range of the distinct characteristics. In some embodiments, the input distinct characteristics are converted to numerical array characteristics, by following a pre-processing method that may include but is not limited to embedding and/or One-Hot encoding and/or BioOne-Hot encoding and/or ensemble preprocessing. Each distinct character of the input amino acid sequences is encoded into a 2-dimensional matrix through one-hot encoding. In other embodiments, each column of the 2-dimensional matrix represents a specific distinct characteristic, and each row corresponds to the position of that distinct characteristic in a sequence. In another embodiment, we used padding and added null character J to normalize the sequences of various lengths into fixed lengths such as length Z. The padding types may include but are not limited to dense padding and sparse padding. The dense paddings may include but are not limited to pre-padding in which null character J is added at the beginning of sequence, post-padding in which null character J is added at the end, mid-padding in which null character J is added in the middle and ext-padding in which null character J is added at both ends. The sparse paddings may include but are not limited to rnd-padding in which null character J is added randomly, strf-padding in which null character J is added uniformly and zoom padding in which amino acids are duplicated. Since there are 24 amino acids (20 standards distinct characteristics (A, R, N, D, C, Q, E, G, H, I, L, K, M, F, P, S, T, W, Y, V), two rare distinct characteristics (U, O), one unknown distinct characteristic (X), and one null distinct characteristic ( )), a CDR-H3 sequence with a length of Z amino acids results in a Z x 24 matrix. Each row includes a single ‘1’ in the column corresponding to the amino acid in its position, whereby this value for all other columns in that row is equal to ‘0’.

In other embodiments, the method comprises interrogating the deep machine-learning engine for a series of distinct characteristic outputs. The distinct characteristic outputs may have attributes similar and/or different to those of the initial input distinct characteristics.

According to some embodiments, the deep machine-learning engine contains deep neural networks. In some embodiments, the deep neural networks are generative models. In another embodiment, the generative model adopts a neural network structure, which includes but is not limited to a residual neural network (Resnet) structure. The Resnet structure, introduces skip connections to jump over some layers. The skip connections allow gradients of a deep neural network to flow easily from layer to layer and prevent gradients from vanishing. In some embodiments, the generative model equipped with Resnet structure is trained by distinct characteristics of different antibody, nanobody, or fragment amino acid sequences for one and/or more target antigens, including but not limited to the complementarity determining regions in the heavy chains (CDR-Hs) and the complementarity determining regions in the light chains (CDR-Ls) and/or the variable regions in the heavy chains (VHs) and the variable regions in the light chains (VLs). These antibody/nanobody amino acid sequences may include but are not limited to CDR-H1, CDR-H2, CDR-H3, CDR-L1, CDR-L2, CDR-L3, FWR-H1, FWR-H2, FWR-H3, FWR-L1, FWR-L2, and FWR-L3 and FWR-L4 sequences to predict antibody, nanobody, and/or fragment or mutations amino acid sequences without vanishing gradient problems. In another embodiment, training data were passed to the generative model equipped with Resnet structure with optimized hyperparameters such as Adam optimizer, ReLu activation, and loss function of total loss=reconstruction loss (binary cross-entropy)+KL-Divergence loss and an optimized learning rate of 0.001. By using Resnet structure, it is possible to converge the deep machine-learning engine of several tens of layers and more, to increase the total accuracy and decrease the total loss of the deep generative model as the number of layers of the deep neural network increases. In other embodiments, the optimized deep generative model with Resnet structure which is trained on a diverse range of amino acid sequences may propose antibody, nanobody, and fragment or mutation amino acid sequences for target antigens that were among the training amino acid sequences. In another embodiment, the deep generative model with Resnet structure which is trained on a diverse range of amino acid sequences may propose antibody, nanobody, fragment, or mutation amino acid sequences for target antigens that were not among the training amino acid sequences. These proposed antibody, nanobody, and fragment, or mutation amino acid sequences may have interactions with the same and/or different target antigens. In some embodiments, the optimized deep generative model with the Resnet structure provides a large in-silico library of biologically valid amino acid sequences that cannot be generated without the Resnet structure.

In some embodiments, the deep learning model uses a combination of reinforcement learning (RL) and a generative adversarial network (GAN) toward improved in-silico design of humanized antibodies, nanobodies and fragments or mutations of variable regions.

In some embodiments, querying a deep machine-learning engine for proposed amino acid sequences and identifying the proposed amino acid sequences are performed consecutively. The method further comprises quality measures comparing the initial antibody amino acid sequences with the proposed amino acid sequences for one and/or more antibodies/nanobodies interacting with one and/or more target antigens.

In some embodiments, the deep learning model is used to perform characterization and modeling of the antibody-paratope:antigen-epitope binding interactions for epitope prediction and identifying antibodies' mechanisms of action using only amino acid sequences.

According to some embodiments, the deep machine-learning engine predicts the antibody/nanobody 3D structures of the generated antibody/nanobody paratope sequences including the 3D structures of the side chains and backbones of antibody/nanobody. The deep machine-learning engine utilizes the 3D structure and the sequences of a given antigen epitope to predict the 3D structure of the generated antibody/nanobody paratopes that bind to the given antigen epitope. In some embodiments, the deep machine-learning engine utilizes an equivariant graph neural network to incorporate the 3D structure of a given antigen epitope to predict antibody paratopes' 3D structure. In another embodiment, the graph neural network models the backbone at the inter-residue geometry levels consisting of inter-residue orientations and distances. In other embodiments, the graph neural network models the surface side chains at the atomic level to incorporate the side-chain conformations under a set of epitope orientations and translations for the antibody paratope 3D structure prediction. In other embodiments, the equivariant graph neural network predicts the paratope 3D structure by modeling the epitope-paratope interactions that may include but are not limited to electrostatic interactions, hydrogen bonds, van der Waals forces, and hydrophobic interactions. The deep generative model improves the antibody paratope 3D structure prediction with accurate side-chain and backbone modeling simultaneously. The deep generative model may also predict the antibody-antigen binding affinity by modeling the antibody paratope-antigen epitope interaction interfaces through the graph neural network.

In some embodiments, querying a deep machine-learning engine for proposed antibody, nanobody, or fragment paratope sequences, their predicted 3D structures, and the antibody, nanobody-antigen binding affinity prediction are performed consecutively. The method comprises quality measures to compare the generated antibody amino acid sequences with the optimized amino acid sequences and their predicted 3D structures for one and/or more antibodies interacting with the given target antigen. The method further comprises quality measures to evaluate the predicted antibody-antigen binding affinity accurately. The method further comprises an automated and multimodal screening method to identify the shortlist of antibodies, nanobodies, or fragments thereof, with drug-like properties.

According to some embodiments, the method further comprises employing a transfer learning technique to solve the data shortage problem for new and/or existing targets lacking sufficient training data. In some embodiments, the transfer learning technique includes but is not limited to a network transfer learning method. In other embodiments, the deep generative model with Resnet structure is trained on sufficient and diverse training data of different antibody amino acid sequences that have interactions with one and/or more target antigens. The method further includes querying the deep generative model with Resnet structure using values derived from the pre-trained deep generative model with Resnet structure to train antibody amino acid sequences lacking sufficient training data to propose antibodies/nanobodies amino acid sequences for novel and/or existing target antigens. In some embodiments, the proposed amino acid sequences for the new and/or existing target antigens lacking sufficient amount of training data may have similar and/or different distinct characteristics to the training data. In another embodiment, the deep generative model with Resnet structure and equipped with transfer learning technique, may propose antibodies/nanobodies amino acid sequences for target antigens that were not among the training amino acid sequences data. These proposed antibody, nanobody, fragment, or mutation amino acid sequences may have interactions with the same and/or different target antigens. In some embodiments, the optimized deep generative model with the Resnet structure and equipped with transfer learning technique provides a large in-silico library of biologically valid amino acid sequences compared to the deep generative model without the Resnet structure and without the transfer learning technique. In another embodiment, the large in-silico library of biologically valid amino acid sequences provided by the deep generative model can be used to design monoclonal, bispecific and trispecific therapeutic antibodies against one and/or multiple target antigens.

In some embodiments, querying a deep machine-learning engine for proposed amino acid sequences and identifying the proposed amino acid sequences for antibody, nanobody, fragment, or mutation amino acid sequences lacking sufficient training data for novel and/or existing target antigens are performed consecutively. The method further comprises quality measures comparing the initial antibody, nanobody, or fragment amino acid sequences with the proposed antibody, nanobody, fragment, or mutation amino acid sequences for one and/or more antibodies interacting with new and/or existing target antigens that lack sufficient training data.

In another embodiment, the method may further provide a short list of the best lead amino acid sequence candidates for new and/or existing target antigens through in-silico screening process. In some embodiments, the method acquires the predicted amino acid sequences and measures the associated biophysical and/or therapeutic properties for each distinct characteristic of antibody, nanobody, fragment, or mutation amino acid sequences against target antigens. In another embodiment, the biophysical and/or therapeutic and/or developability properties for therapeutic developability may include but are not limited to similarity, diversity, hydrophobicity, net-charge, isoelectric point, solubility, stability, immunogenicity, viscosity, clearance, specificity, affinity, affinity maturation, cross-reactivity, developability, manufacturability, half-life, pharmacokinetic, yield, aggregation, function, antibody epitope mapping, and antibody humanness and humanization of amino acid sequences against one and/or more target antigens.

In other embodiments, the method comprises an ensemble model that utilizes the biological and key therapeutic property values offered by our deep-learning model to identify a shortlist of antibody therapeutics with optimal drug-like properties. The method further comprises an automated and multimodal screening model to identify novel drug candidates. The multimodal screening approach has the capacity to simultaneously capture the total biophysical and therapeutic properties space of antibodies, nanobodies, fragments, or mutations to select the best antibody, nanobody, or fragment lead candidates faster.

In some embodiments, in-silico screening of the proposed antibody, nanobody, fragment, or mutation amino acid sequences for novel and/or existing target antigens is performed, consecutively. The method further comprises quality measures comparing the initial antibody amino acid sequences with the proposed amino acid sequences for one and/or more antibodies/nanobodies interacting with new and/or existing target antigens that may or may not lack sufficient training data to find the most developable therapeutic candidates.

According to some embodiments, the deep machine-learning engine comprising a deep learning and feature learning method for analyzing high-dimensional data in form of amino acid sequences against new and/or existing target antigens is provided. The amino acid sequences may include but are not limited to biological antibody/nanobody amino acid sequences and/or in-silico generated antibody, nanobody, or fragment amino acid sequences. In another embodiment, the deep learning-based method adopts Resnet structures. In some embodiments, the deep learning-based method is used for dimensionality reduction visualization. In other embodiments, the deep learning-based method transforms mappings from a high-dimensional data space into a lower-dimensional feature space, leading to visualization of antibody amino acid sequences in two and/or more dimensions. In another embodiment, the deep learning-based method is used to improve cluster analysis. In other embodiments, the deep learning-based method is used to recognize the hidden patterns in the amino acid sequences spaces. In another embodiment, the deep learning-based method is used to repurpose the amino acid sequences for one target recognized within the hidden patterns sharing the same and/or similar latent space with another target for other targets and/or different targets. In other embodiments, the deep learning-based method is formulated based on the learned and extracted features. The method may include querying the deep learning-based method for the latent encodings of the input training data to follow a standard normal distribution, wherein each input data has clear boundaries. In some embodiments, the deep learning-based method groups the output distinct characteristics in one class when they have attribute values close to each other in the latent space. The distinct characteristics with similar attributes are grouped in one cluster. The distinct characteristics with different attributes are grouped in different cluster. The distinct characteristics with similar and/or different attributes are grouped in shared latent space clusters. According to some embodiments, the deep learning-based method can recognize patterns, which exist between and/or within the clusters and/or between the clusters with the shared latent spaces. The clusters represent latent spaces associated with target antigens and the distinct characteristics of each cluster represent antibody, nanobody, or fragment amino acid sequences. The deep learning-based method may further effectively recognize and/or may propose antibodies, nanobodies, or fragments for target antigens from each cluster. The deep learning-based method may further recognize antibody, nanobody, or fragment amino acid sequences based on their biophysical property of specificity to one and/or multiple target antigens. In another embodiment, the deep learning-based method may further recognize antibody, nanobody, or fragment amino acid sequences based on their biophysical property of cross-reactivity to one and/or multiple target antigens. In other embodiments, the deep learning-based method comprises identifying antibody, nanobody, or fragment amino acid sequences, which are sharing the same latent space across different clusters. The deep learning-based method may recognize the hidden patterns across the antibody, nanobody, or fragment amino acid sequences spaces. The deep learning-based method may further repurpose antibody, nanobody, or fragment amino acid sequences that are common across different clusters for new and/or existing target antigens. The deep learning-based method may further repurpose antibody, nanobody, or fragment amino acid sequences that are common across different clusters for multiple target antigens. In some embodiments, the method may map the paratopes of the antibodies, nanobodies, or fragment for their binding to the target antigen epitopes.

In some embodiments, querying a deep learning-based method for dimensionality reduction of the antibody, nanobody, or fragment amino acid sequences, and/or for improving the clustering of the antibody, nanobody, or fragment amino acid sequences, and/or for finding the hidden patterns of the antibody, nanobody, or fragment amino acid sequences, and/or repurposing the antibody, nanobody, or fragment amino acid sequences for novel and/or existing target antigens and/or analyzing the antibody, nanobody, or fragment amino acid sequences for novel and/or existing target antigens are performed consecutively. The deep learning-based method further comprises quality measures comparing the amino acid sequences in each cluster and the amino acid sequences sharing the same and/or similar and/or different latent spaces. The deep learning-based method proposes amino acid sequences for one and/or more antibodies, nanobodies, or fragments thereof, interacting with new and/or existing target antigens and/or one or multiple target antigens.

In some embodiments, the method further comprises classifying antibodies, nanobodies, or fragments thereof, based on their binding capabilities. In some embodiments, the classifying comprises use of a deep neural network (DNN).

In some embodiments, our deep learning-based method further uses BioOne-Hot encoding with biophysiochemical values for improving the antibody amino acid sequences' classification models.

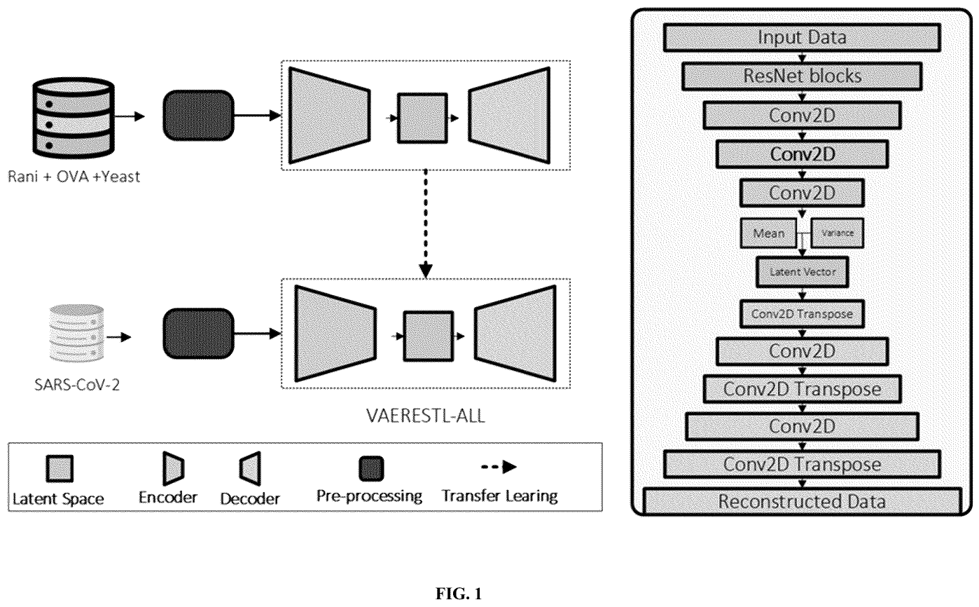

FIG. 1 illustrates an overview representation of an example system that identifies proposed amino acid sequences using a variational autoencoder (VAE) Resnet transfer learning (VAEResTL) deep generative model trained on initial amino acid sequence(s). CDR-H3 sequences are pre-processed to have the same length, and then are converted to numerical arrays. Then, the VAE Resnet (VAERes) model is trained with pre-processed data. Due to the shortage of data in the SARS-CoV-2 dataset, The VAERes is first trained on a larger CDR-H3 sequence dataset. To show the generality of the method, VAERes is trained on four datasets: Ranibizumab, OVA, Yeast display scFV displayed scFv, and all three mentioned targets together, labeled as ALL. Finally, the transfer learning technique is used to train the VAERes model on small dataset of SARS-CoV-2. FIG. 1 also illustrates the structure of the exemplary VAEResTL deep generative method for identifying a proposed amino acid sequence(s). The VAERes model comprises an encoder and a decoder. The encoder includes residual, convolutional, and dense layers. Residual layers are used to learn features better while preventing over fitting and vanishing gradient problems. The output of the encoder is a latent vector which is calculated with a mean and a variance. The decoder utilizes convolutional and convolutional transpose layers to reconstruct the input from the latent vector.

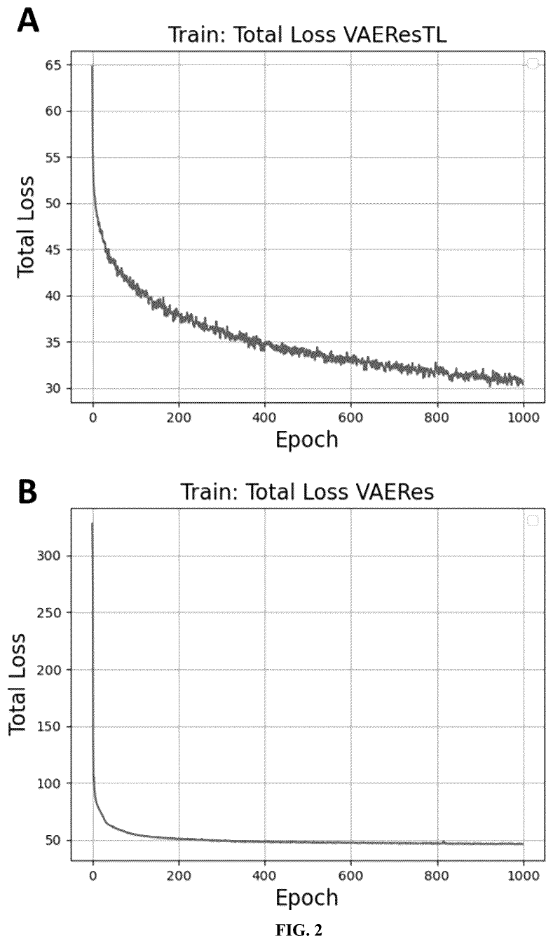

FIG. 2A is a graph demonstrating the total loss during training for the VAEResTL model.

FIG. 2B is a graph demonstrating the total loss during training for the VAERes model.

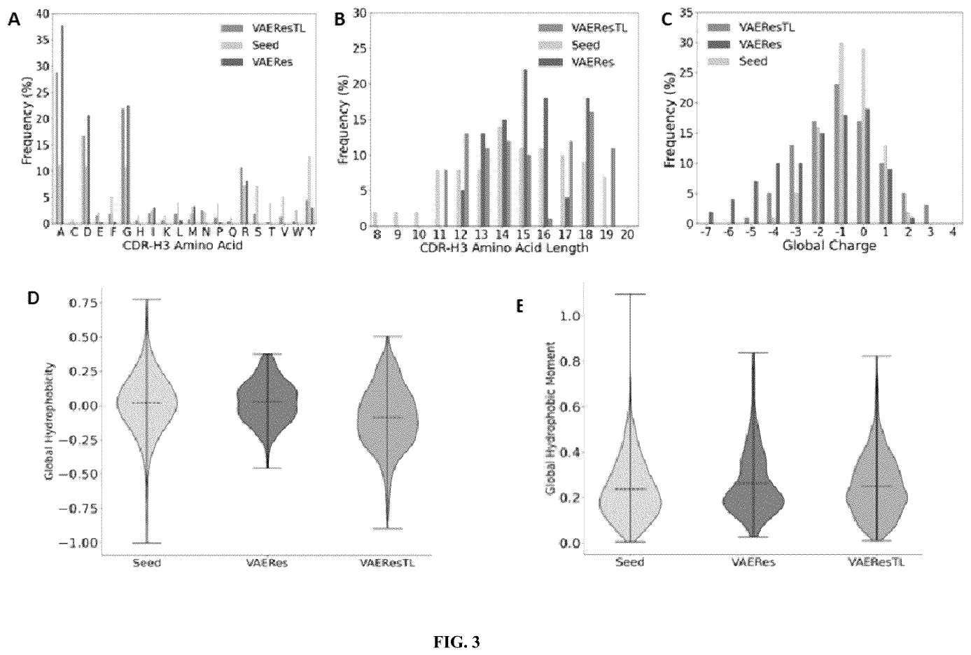

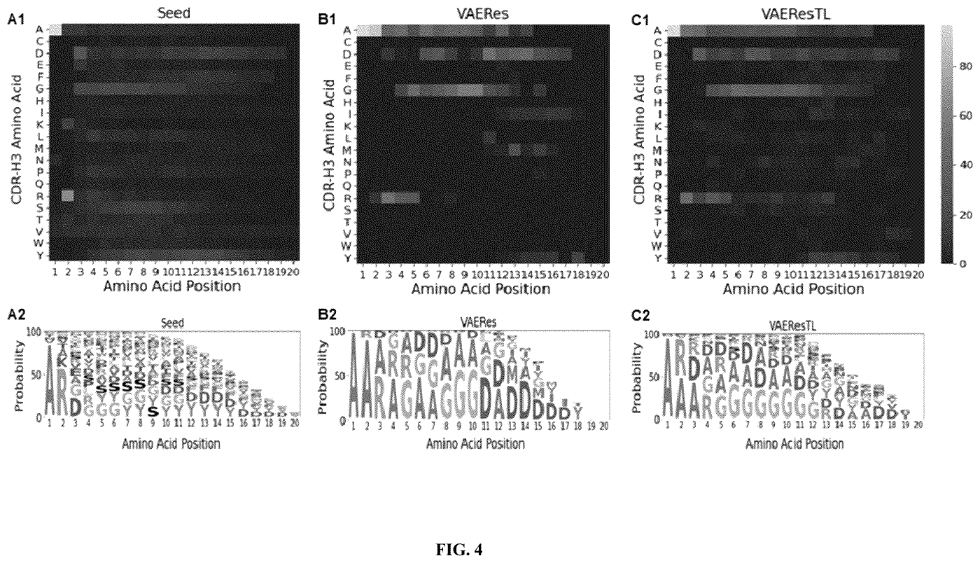

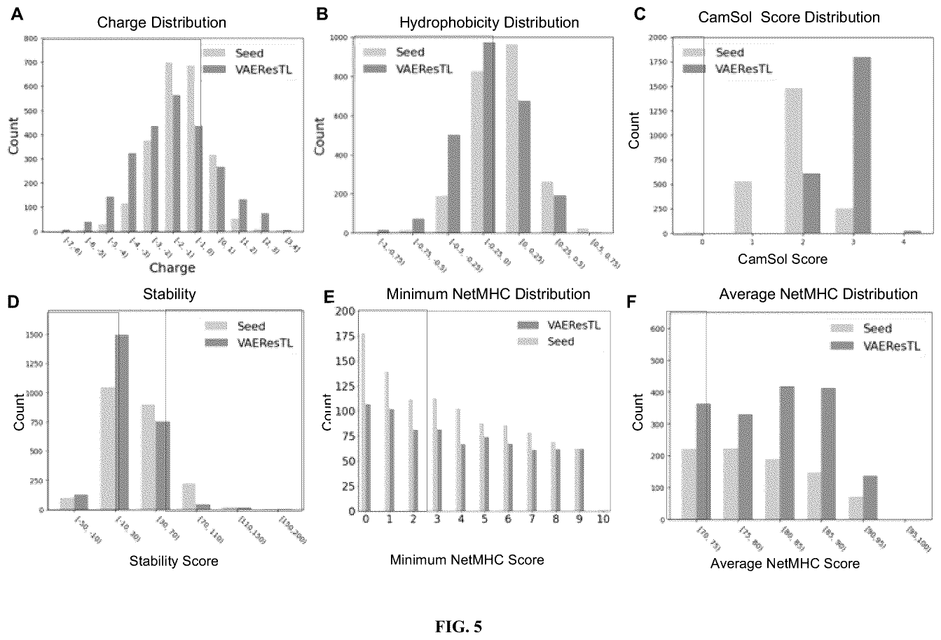

FIG. 3A provides a Visualize Comparison of molecular characteristics, amino acid distribution, between Seed (SARS-CoV-2 seed sequences, Yellow), VAERes (generated sequences by VAERes for SARS-CoV-2, Violet) and VAEResTL (generated sequences by VAEResTL for SARS-CoV-2, Turquoise). Horizontal dashed lines account for the mean. Whiskers extend to the most extreme non-outlier data points.

FIG. 3B provides a Visualize Comparison of molecular characteristics, amino acid length distribution, between Seed (SARS-CoV-2 seed sequences, Yellow), VAERes (generated sequences by VAERes for SARS-CoV-2, Violet) and VAEResTL (generated sequences by VAEResTL for SARS-CoV-2, Turquoise). Horizontal dashed lines account for the mean. Whiskers extend to the most extreme non-outlier data points.

FIG. 3C provides a Visualize Comparison of molecular characteristics, total charge distribution, between Seed (SARS-CoV-2 seed sequences, Yellow),VAERes (generated sequences by VAERes for SARS-CoV-2, Violet) and VAEResTL (generated sequences by VAEResTL for SARS-CoV-2, Turquoise). Horizontal dashed lines account for the mean. Whiskers extend to the most extreme non-outlier data points.

FIG. 3D provides a Visualize Comparison of molecular characteristics, Eisenberg hydrophobicity, between Seed (SARS-CoV-2 seed sequences, Yellow),VAERes (generated sequences by VAERes for SARS-CoV-2, Violet) and VAEResTL (generated sequences by VAEResTL for SARS-CoV-2, Turquoise). Horizontal dashed lines account for the mean. Whiskers extend to the most extremenon-outlier data points.

FIG. 3E provides a Visualize Comparison of molecular characteristics, Eisenberg Hydrophobic moment, between Seed (SARS-CoV-2 seed sequences, Yellow),VAERes (generated sequences by VAERes for SARS-CoV-2, Violet) and VAEResTL (generated sequences by VAEResTL for SARS-CoV-2, Turquoise). Horizontal dashed lines account for the mean. Whiskers extend to the most extremen on-outlier data points.

FIG. 4A1 is a Machine Learning (ML) Visualization that demonstrates a position proposed map. Heatmap visualization showing the count of observed seed sequences for SARS-CoV-2.

FIG. 4B1 is a Machine Learning (ML) Visualization that demonstrates a position proposed map. Heatmap visualization showing the count of VAERes-generated sequences or SARS-CoV-2 from (left) and to (right) each amino acid at each sequence position.

FIG. 4C1 is a Machine Learning (ML) Visualization that demonstrates a position proposed map. Heatmap visualization showing the count of VAEResTL-generated sequences for SARS-CoV-2 from (left) and to (right) each amino acid at each sequence position (seed and VAERes-generated and VAEResTL-generated sequences having length 20). Sequence logo visualizations.

FIG. 4A2 is a Machine Learning (ML) Visualization that demonstrates sequence logos for Seeds SARS-CoV-2.

FIG. 4B2 is a Machine Learning (ML) Visualization that demonstrates the VAERes-generated sequences for SARS-CoV-2.

FIG. 4C2 is a Machine Learning (ML) Visualization that demonstrates the VAEResTL-generated sequences for SARS-CoV-2 are based on residue frequency. Sequence logos are computed using Skylign.

FIG. 5A illustrates In-Silico Screening of predicted CDR-H3 sequences for further assessment, Net charge of CDR-H3 sequence. Histograms present the parameter distributions of all generated and seed sequences for different filtering steps. Boxes show filtering cut-off for the therapeutic index in clinical setting.

FIG. 5B illustrates In-Silico Screening of predicted CDR-H3 sequences for further assessment, CDR-H3 hydrophobicity. Histograms present the parameter distributions of all generated and seed sequences for different filtering steps. Boxes show filtering cut-off for the therapeutic index in clinical setting.

FIG. 5C illustrates In-Silico Screening of predicted CDR-H3 sequences for further assessment, CamSol solubility score. Histograms present the parameter distributions of all generated and seed sequences for different filtering steps. Boxes show filtering cut-off for the therapeutic index in clinical setting.

FIG. 5D illustrates In-Silico Screening of predicted CDR-H3 sequences for further assessment, CDR-H3 stability score. Histograms present the parameter distributions of all generated and seed sequences for different filtering steps. Boxes show filtering cut-off for the therapeutic index in clinical setting.

FIG. 5E illustrates In-Silico Screening of predicted CDR-H3 sequences for further assessment, The minimum NetMHCIIpan % Rank (<2 strong binder; <10 weak binder) across all possible 15-mers for a given CDR-H3 sequences and across all 26 HLA alleles. Histograms present the parameter distributions of all generated and seed sequences for different filtering steps. Boxes show filtering cut-off for the therapeutic index in clinical setting.

FIG. 5F illustrates In-Silico Screening of predicted CDR-H3 sequences for further assessment, The average NetMHCIIpan % Rank across all possible 15-mers for a given CDR-H3 sequences and across all 26 HLA alleles. Histograms present the parameter distributions of all generated and seed sequences for different filtering steps. Boxes show the filtering cut-off for the therapeutic index in a clinical setting.

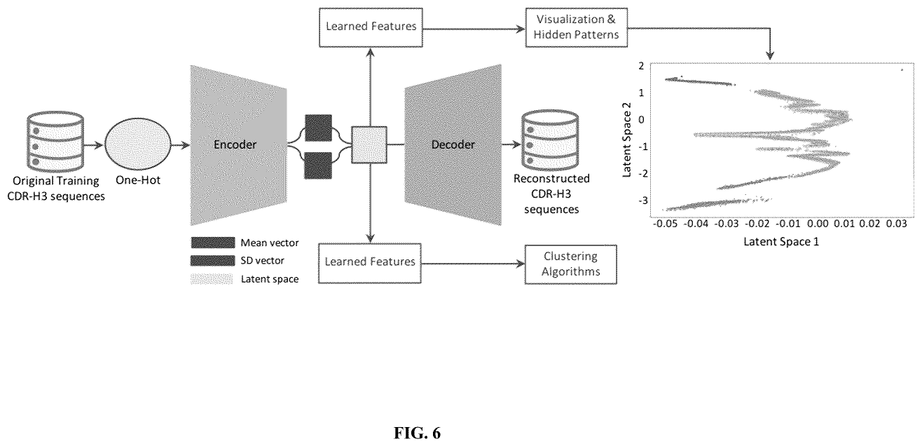

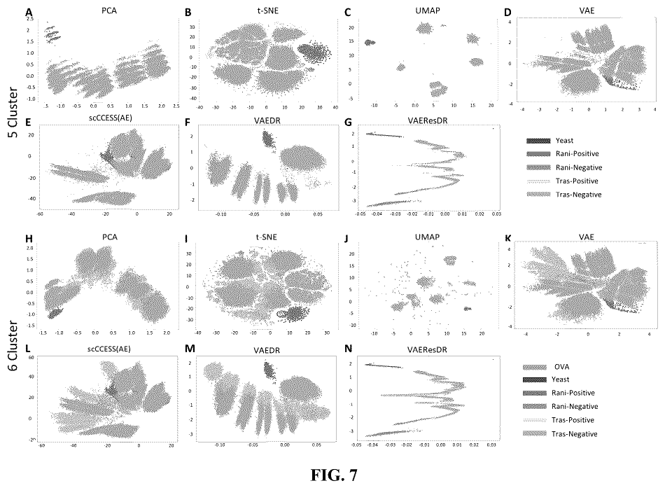

FIG. 6 illustrates an overview representation of an example system that identifies amino acid sequences based on their single or multiple target specificity and/or antibody amino acid sequences' cross-reactivity to one or multiple targets using a variational autoencoder (VAE) Resnet dimensionality reduction (VAEResDR) model, which is a deep learning-based feature extractor model trained on amino acid sequence(s). CDR-H3 sequences are pre-processed to have the same length, and then converted to numerical arrays. Then, the VAEResDR model is trained with pre-processed data. To show the generality of the method, VAEResDR is trained on four datasets: Ranibizumab, OVA, Yeast display scFV displayed scFv, and Trastuzumab. The VAEResDR model comprises an encoder and a decoder. The encoder scales down high-dimensional input sequences into a two-dimensional feature space, and the decoder reconstructs the sequences from these two-dimensional features. The extracted features by the VAEResDR model are used for two-dimensional visualization presentation of antibody amino acid sequences. Moreover, the extracted features by the VAEResDR model are utilized to improve the clustering of antibody amino acid sequences.

FIG. 7A is a visualization plot of dimensionality reduction (DR) for the baseline PCA. CDR-H3 sequences for three target antigens in 5 clusters were processed.

FIG. 7B is a visualization plot of dimensionality reduction for the baseline t-SNE. CDR-H3 sequences for three target antigens in 5 clusters were processed.

FIG. 7C is a visualization plot of dimensionality reduction for the baseline UMAP. CDR-H3 sequences for three target antigens in 5 clusters were processed.

FIG. 7D is a visualization plot of dimensionality reduction for the baseline VAE. CDR-H3 sequences for three target antigens in 5 clusters were processed.

FIG. 7E is a visualization plot of dimensionality reduction for the baseline scCCESS (AE). CDR-H3 sequences for three target antigens in 5 clusters were processed.

FIG. 7F is a visualization plot of dimensionality reduction for the baseline VAEDR. CDR-H3 sequences for three target antigens in 5 clusters were processed.

FIG. 7G is a visualization plot of dimensionality reduction for the VAEResDR. CDR-H3 sequences for three target antigens in 5 clusters were processed.

FIG. 7H is a visualization plot of dimensionality reduction for baseline PCA. CDR-H3 sequences for four target antigens in 6 clusters were processed.

FIG. 7I is a visualization plot of dimensionality reduction for baseline t-SNE. CDR-H3 sequences for four target antigens in 6 clusters were processed.

FIG. 7J is a visualization plot of dimensionality reduction for baseline uniform manifold approximation and projection (UMAP). CDR-H3 sequences for four target antigens in 6 clusters were processed.

FIG. 7K is a visualization plot of dimensionality reduction for baseline VAE. CDR-H3 sequences for four target antigens in 6 clusters were processed.

FIG. 7L is a visualization plot of dimensionality reduction for baseline scCCESS (AE). CDR-H3 sequences for four target antigens in 6 clusters were processed.

FIG. 7M is a visualization plot of dimensionality reduction for the baseline VAEDR. CDR-H3 sequences for four target antigens in 6 clusters were processed.

FIG. 7N is a visualization plot of dimensionality reduction for the VAEResDR. CDR-H3 sequences for four target antigens in 6 clusters were processed.

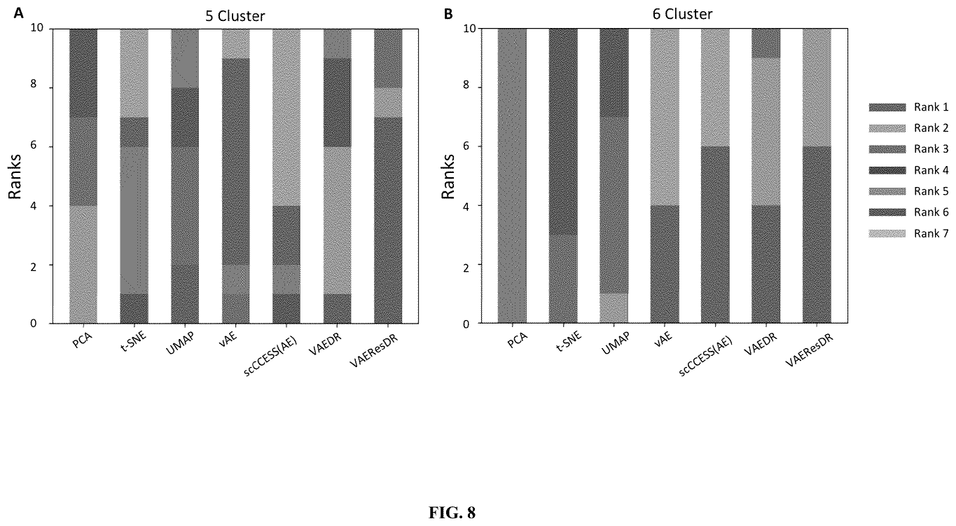

FIG. 8A is a bar chart showing Clustering Comparison to demonstrate the dimensionality reduction (DR) methods' performance on CDR-H3 sequences in 5 clusters. The best performance is ranked 1 and the worst performance is ranked 7. The color codes for rank 1, 2, 3, 4, 5, 6, and 7 are blue, orange, green, red, purple, brown, and pink consecutively.

FIG. 8B is a bar chart showing Clustering Comparison to demonstrate the DR methods' performance on CDR-H3 sequences in 6 clusters. The best performance is ranked 1 and the worst performance is ranked 7. The color codes for rank 1, 2, 3, 4, 5, 6, and 7 are blue, orange, green, red, purple, brown, and pink consecutively.

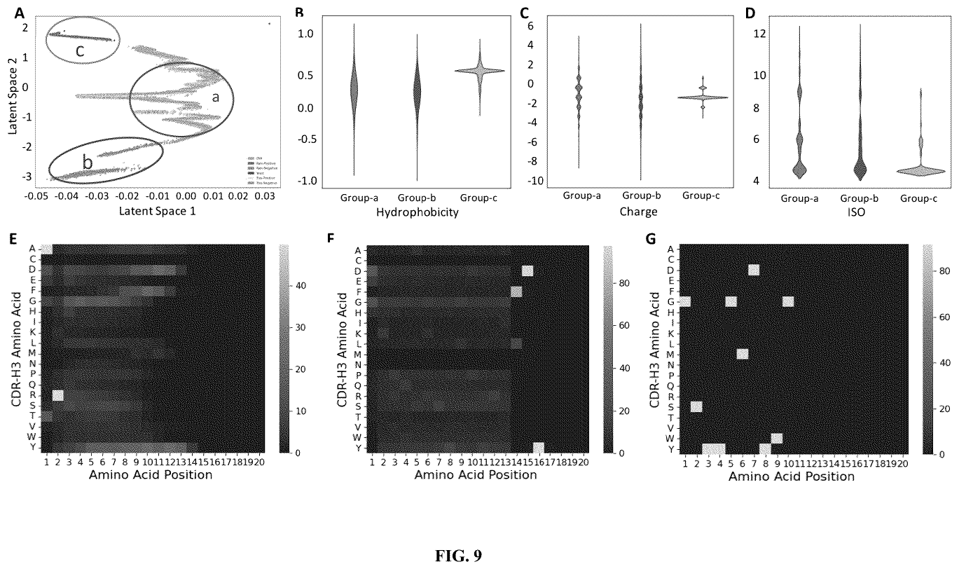

FIG. 9A illustrates a VAEResDR two-dimensional Hidden Patterns Analysis. 2-dimensional visualization by VAEResDR method for four targets, 6 clusters. CDR-H3 Sequences sharing the same latent space are marked as group (a) and CDR-H3 sequences in different latent spaces are marked as group (b) and (c).

FIG. 9B illustrates a VAEResDR two-dimensional Hidden Patterns Analysis, including the biophysical metric of hydrophobicity (H).

FIG. 9C illustrates a VAEResDR two-dimensional Hidden Patterns Analysis, including the biophysical metric of net-charge.

FIG. 9D illustrates a VAEResDR two-dimensional Hidden Patterns Analysis, including the biophysical metric of Iso-electric Point (ISO).

FIG. 9E illustrates a VAEResDR two-dimensional Hidden Patterns Analysis, including the machine learning (ML) visualization Heatmap plot for group (a).

FIG. 9F illustrates a VAEResDR two-dimensional Hidden Patterns Analysis, including the ML visualization Heatmap plot for group (b).

FIG. 9G illustrates a VAEResDR two-dimensional Hidden Patterns Analysis, including the ML visualization Heatmap plot for group (c).

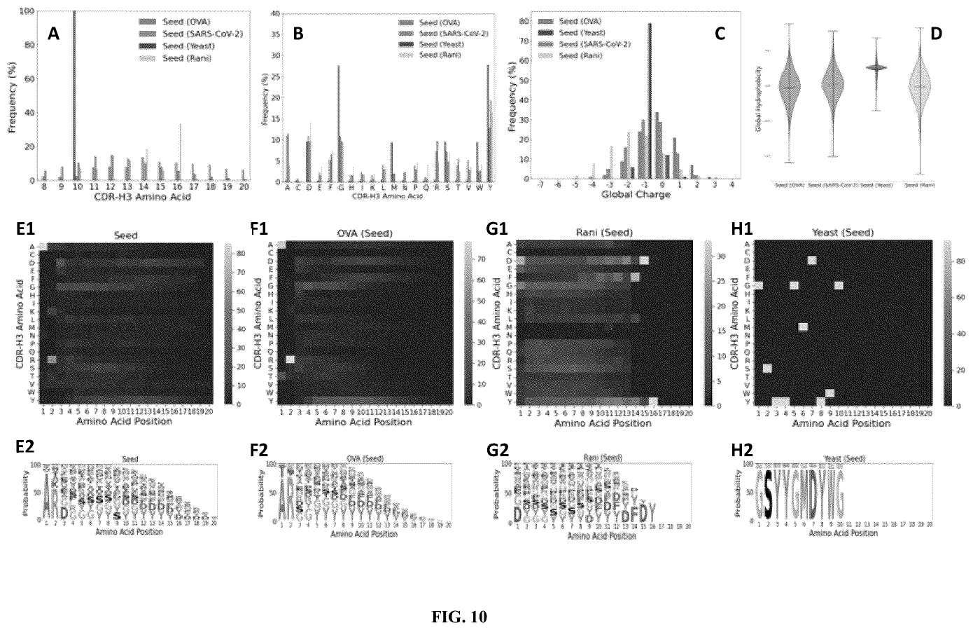

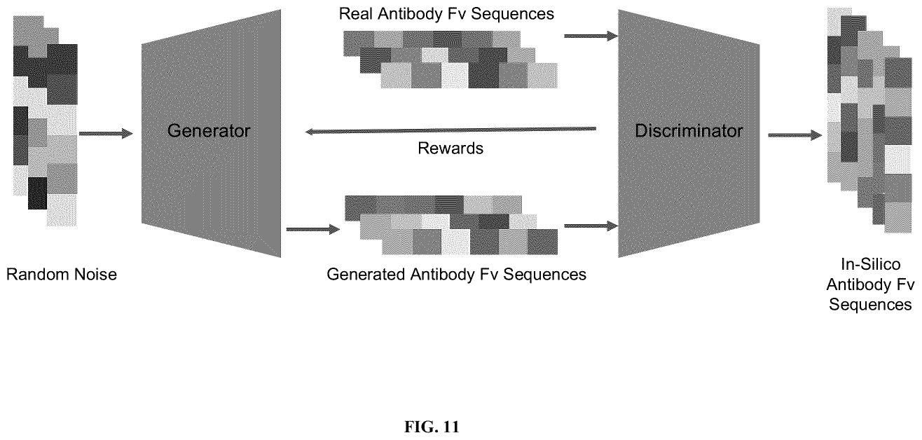

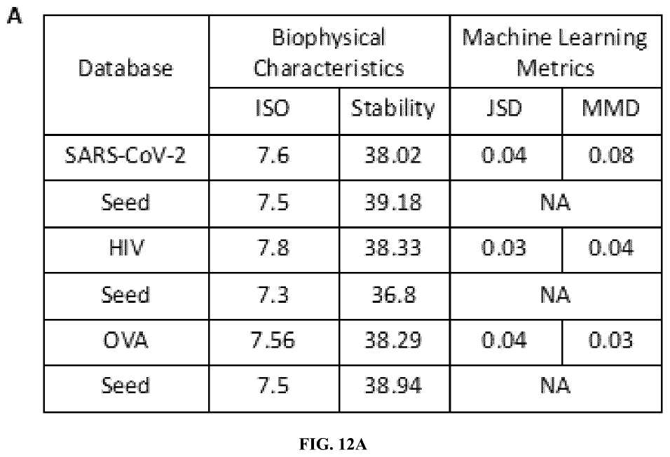

FIG. 10A is an amino acid distribution plot comparing Seed sequences (SARS-CoV-2 seed sequences, orange), (OVA seed sequences, Turquoise), (Yeast display scFV seed sequences, Violet), (Rani seed sequences, Yellow), and includes a schematic of the comparison of the training seed amino acid sequences.

FIG. 10B is an amino acid length distribution plot comparing Seed sequences (SARS-CoV-2 seed sequences, orange), (OVA seed sequences, Turquoise), (Yeast display scFV seed sequences, Violet), (Rani seed sequences, Yellow), and includes a schematic of comparison of the training seed amino acid sequences, and a schematic of the comparison of the training seed amino acid sequences.

FIG. 10C is a total charge distribution plot comparing seed sequences (SARS-CoV-2 seed sequences, orange), (OVA seed sequences, Turquoise), (Yeast display scFV seed sequences, Violet), (Rani seed sequences, Yellow), and includes a schematic of comparison of the training seed amino acid sequences. It demonstrates a schematic of the comparison of the training seed amino acid sequences.

FIG. 10D is an Eisenberg hydrophobicity plot comparing seed sequences (SARS-CoV-2 seed sequences, orange), (OVA seed sequences, Turquoise), (Yeast display scFV seed sequences, Violet), (Rani seed sequences, Yellow), and includes a schematic of comparison of the training seed amino acid sequences, and a schematic of the comparison of the training seed amino acid sequences.

FIG. 10E1 is a heatmap visualization plot for SARS-CoV-2 (Seed) CDR-H3 sequences.

FIG. 10E2 is a logo visualization plot for SARS-CoV-2 (Seed) CDR-H3 sequences.

FIG. 10F1 is a heatmap visualization plot for OVA (Seed) CDR-H3 sequences.

FIG. 10F2 is a logo visualization plot for OVA (Seed) CDR-H3 sequences, a schematic on how the training sequences are distributed.

FIG. 10G1 is Heatmap visualization plot for Rani (Seed) CDR-H3 sequences.

FIG. 10G2 is a Logo visualization plot for Rani (Seed) CDR-H3 sequences, including a schematic on how the training sequences are distributed.

FIG. 101 is a Heatmap visualization plot for Yeast display scFV (Seed) CDR-H3 sequences.

FIG. 10112 is a Logo visualization plot for Yeas (Seed) CDR-H3 sequences, including a schematic on how the training sequences are distributed.

FIG. 11 shows an overview of the AbGAN method.

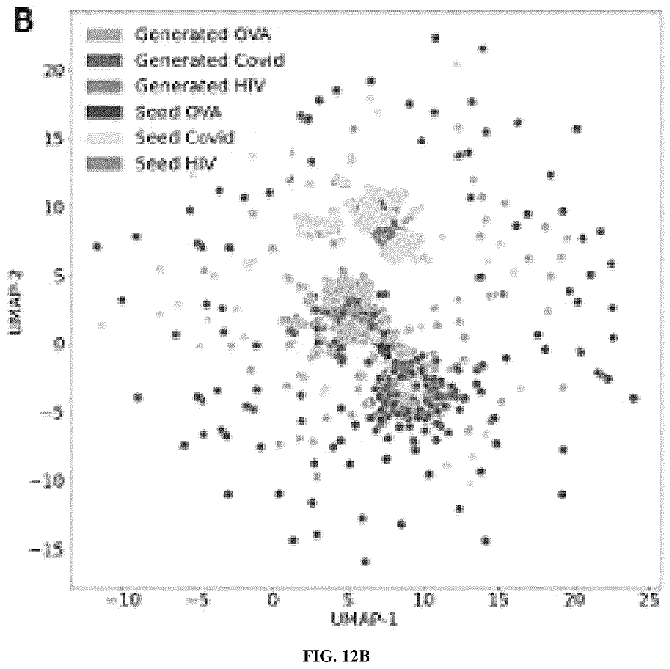

FIG. 12A shows Biological Characteristics and Machine Learning Metrics for AbGAN Fv generated SARS-CoV-2, HIV, and OVA sequences compared to Seed Fv SARS-CoV-2, HIV, and OVA sequences.

FIG. 12B shows UMAP visualization plot illustrating the AbGAN Fv generated SARS-CoV-2, HIV, and OVA sequences compared to Seed Fv SARS-CoV-2, HIV, and OVA sequences in a two-dimensional latent space.

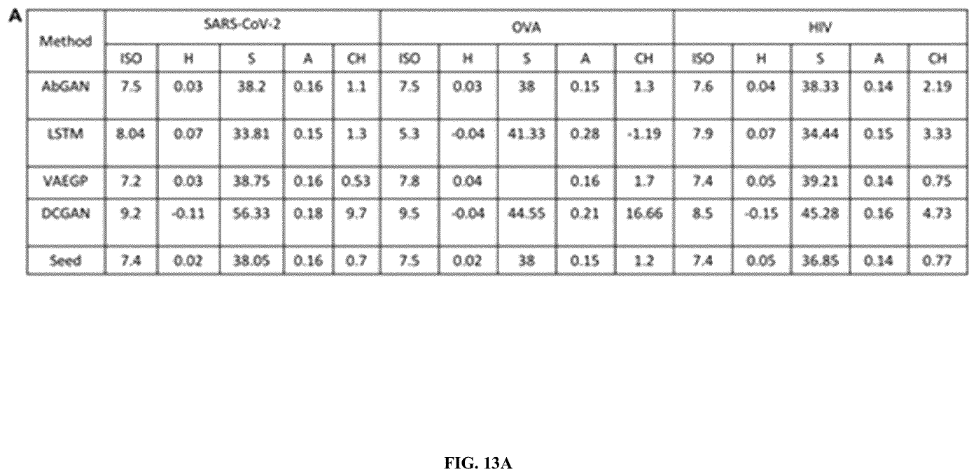

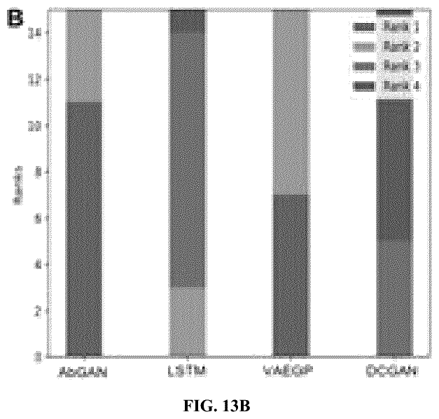

FIGS. 13A-13B show Bar Chart Generative Models Performance Comparison, including: (FIG. 13A) Biological Characteristics Metrics for AbGAN, LSTM, VAEGP, and DCGAN Fv generated SARS-CoV-2, HIV, and OVA sequences compared to Seed Fv SARS-CoV-2, HIV, and OVA sequences; and (FIG. 13B). The best performance is ranked 1 and the worst performance is ranked 4. The color codes for rank 1, 2, 3, and 4 are blue, orange, green, and red consecutively.

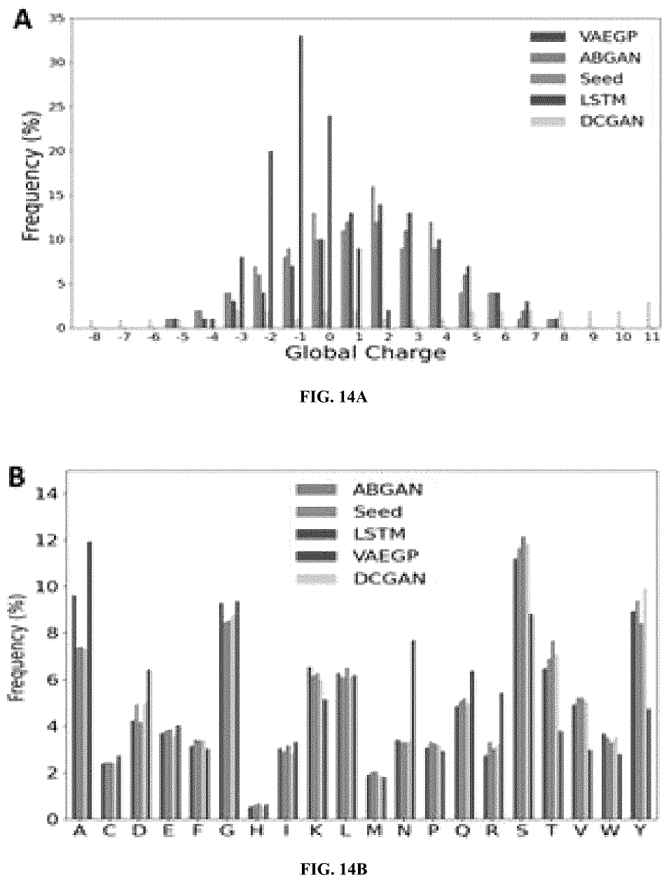

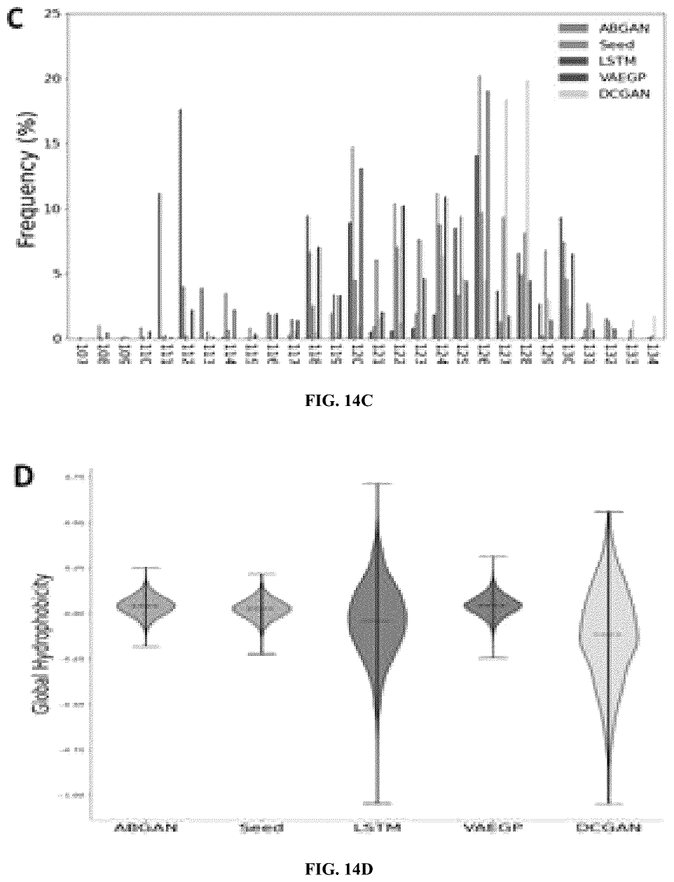

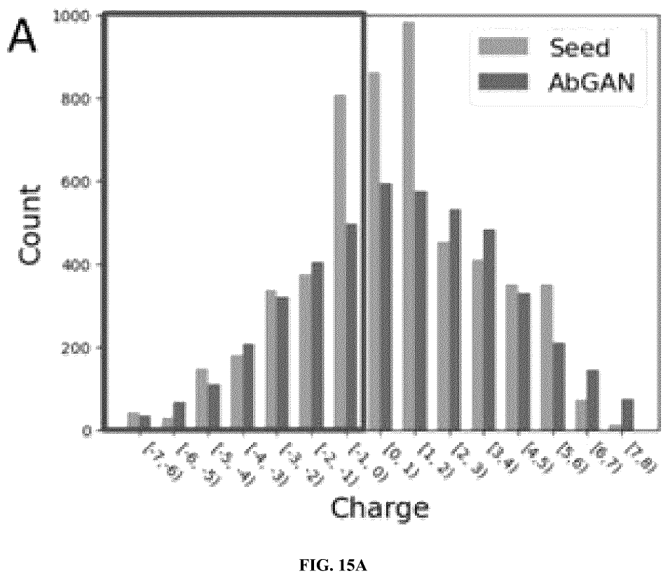

FIG. 14A-14D show Visualize Comparison of molecular characteristics between Seed (SARS-CoV-2 seed sequences, Orang), AbGAN (AbGAN-generated sequences for SARS-CoV-2, Torque) and LSTM (LSTM-generated sequences for SARS-CoV-2, Purple), VAEGP (VAEGP-generated sequences for SARS-CoV-2, Pink), and DCGAN (DCGAN-generated sequences for SARS-CoV-2, Yellow). Horizontal dashed lines account for the mean. Whiskers extend to the most extreme non-outlier data points. The figures include (FIG. 14A) total charge distribution, (FIG. 14B) amino acid distribution, (FIG. 14C) amino acid length distribution, and (FIG. 14D) Eisenberg hydrophobicity.

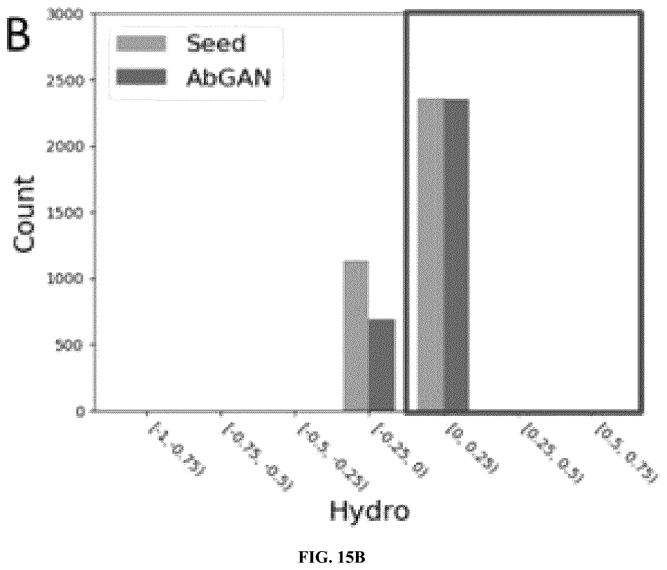

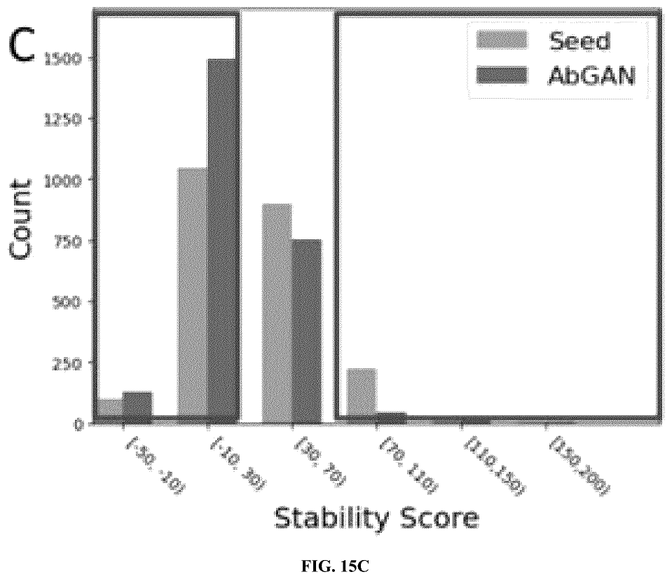

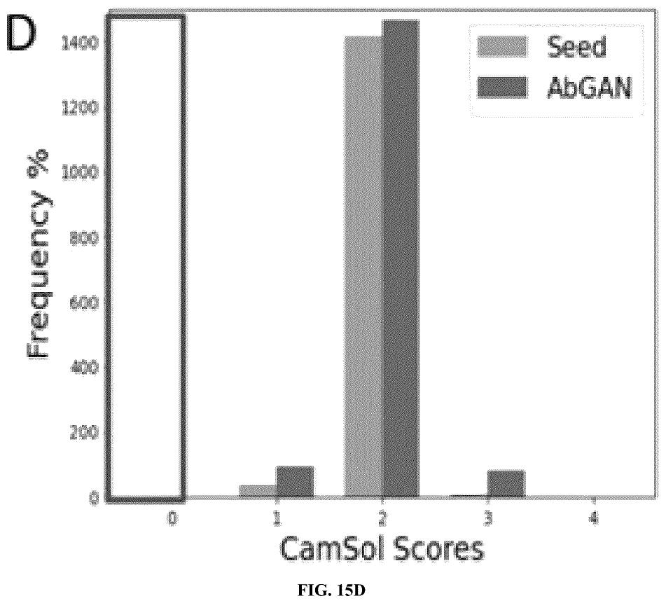

FIG. 15A-15D show In-Silico Screening of predicted Fv sequences. Histograms present the parameter distributions of AbGAN-generated and seed SARS-CoV-2 sequences for different filtering steps. Boxes show the filtering cut-off for the therapeutic index in a clinical setting. The figures include (FIG. 15A) Fv sequences net-charge, (FIG. 15B) Fv sequences H, (FIG. 15C) Fv sequences stability score, and (FIG. 15D) CamSol solubility score.

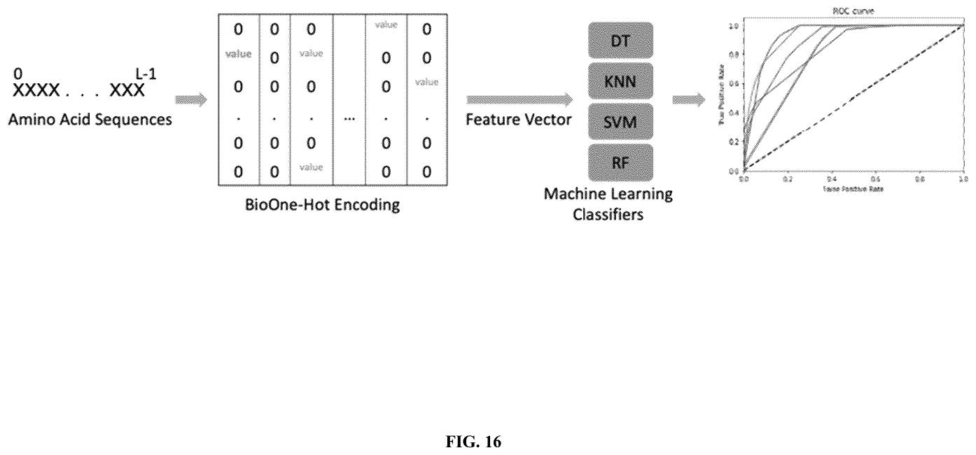

FIG. 16 shows an overview of the method in antibody classification.

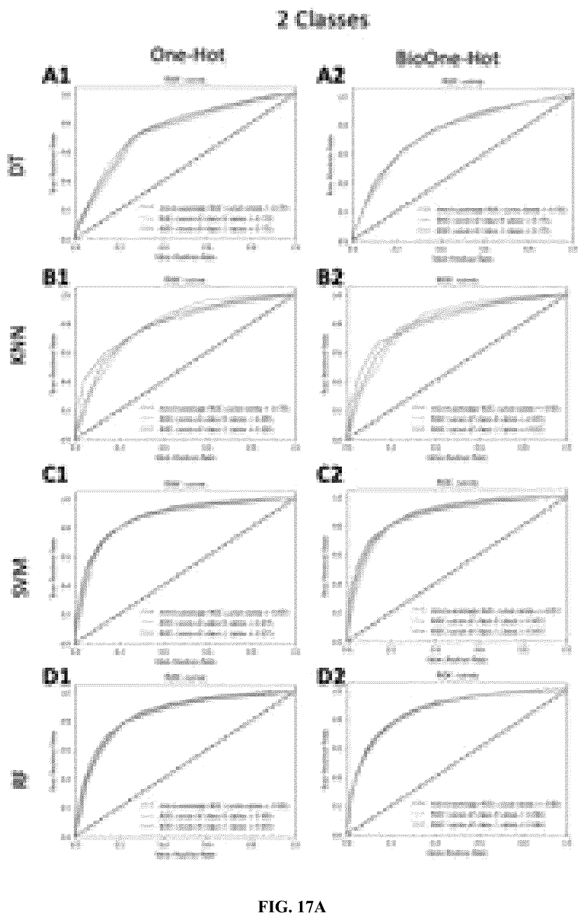

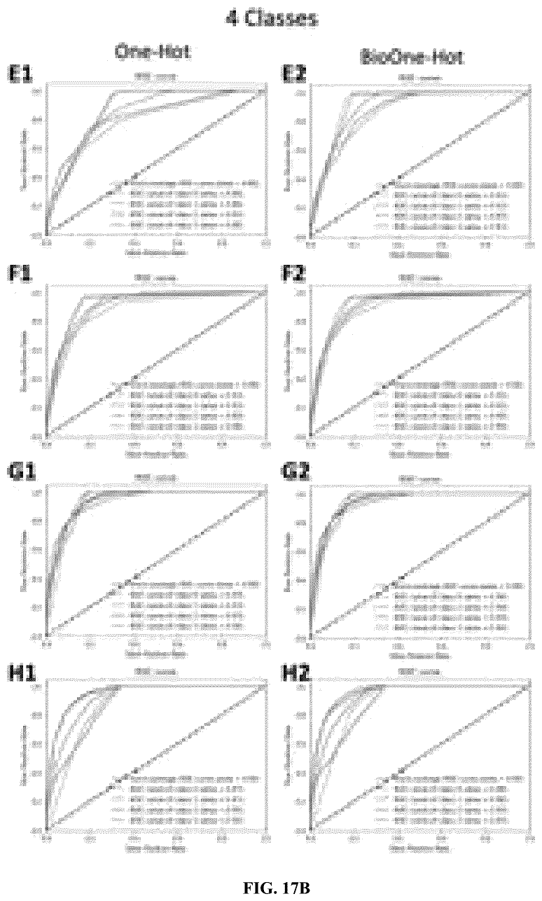

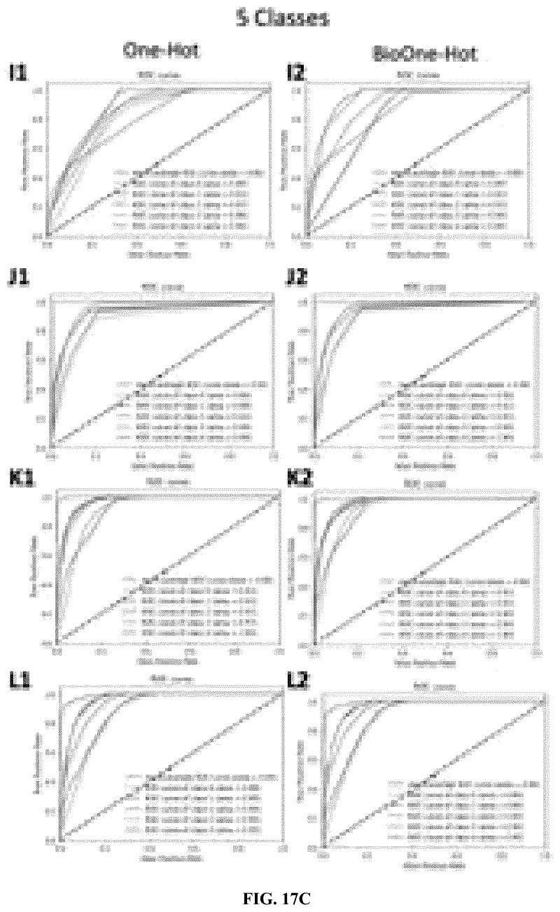

FIG. 17A-17C show receiver-operating characteristic (ROC) Plots. The ROC curves with positive samples threshold at 1.0. Four classification methods of decision tree (DT), K-nearest-neighbor (KNN), support vector machine (SVM), and random forest (RF) were tested on: (FIG. 17A) 2 classes of Tras-Positive and Tras-Negative; (FIG. 17B) 4 classes of Tras-Positive, Tras-Negative, Rani-Positive, and Rani-Negative; and (FIG. 17C) 5 classes of OVA, Tras-Positive, Tras-Negative, Rani-Positive, and Rani-Negative.

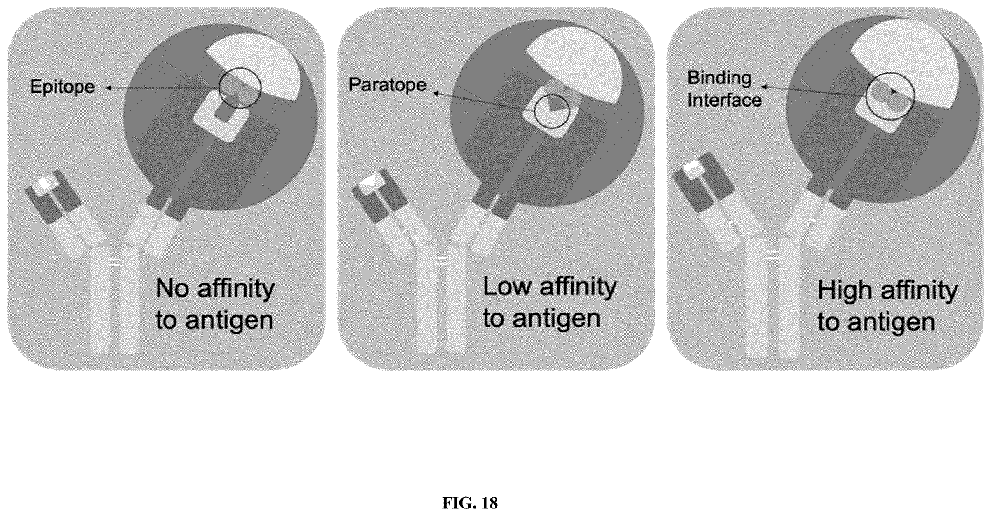

FIG. 18 illustrates an example of antibody paratope matching with antigen epitope. No match between epitope-paratope residues results in no affinity to antigen. Poor matches between epitope-paratope residues result in low affinity to antigen, and high-quality matches between epitope-paratope residue results in a high affinity to antigen.

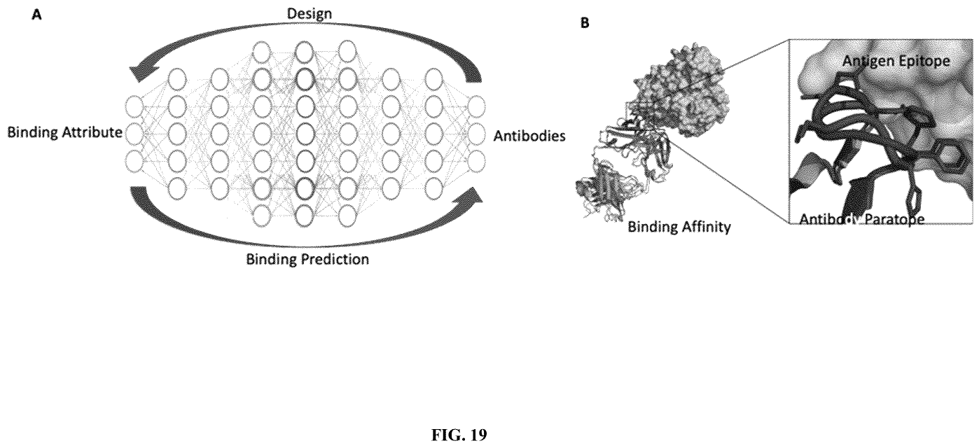

FIG. 19 illustrates an example of an overview of a Deep Learning Model that is developed and trained by antigen paratope and 3D structure to design and predict antibody paratope and 3D structure by accurately modeling the epitope-paratope interactions.

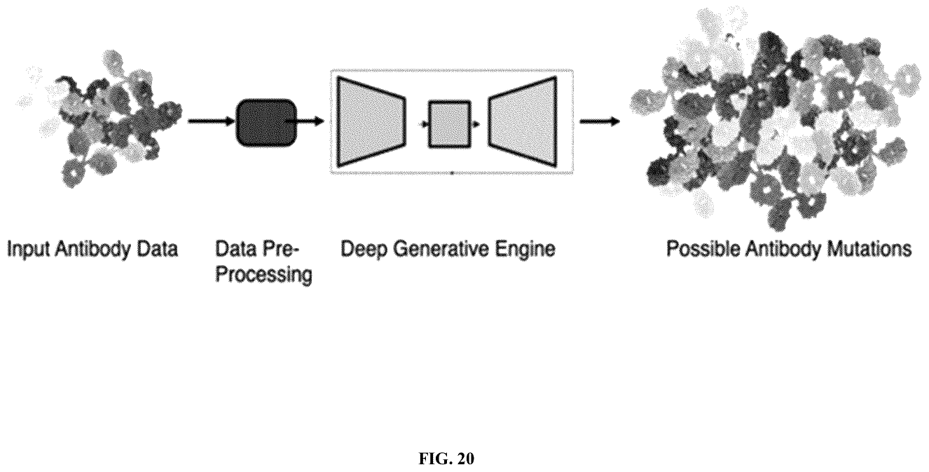

FIG. 20 illustrates that the VAEResTL, a deep generative model, is an end-to-end trainable model which comprises a convolutional Variational Autoencoder (VAE) that adopts a Resnet structure and is enhanced with a Transfer Learning (TL) technique. The method utilizes only the amino acid sequence data without the need for structural data and designs a large library of antibody mutations.

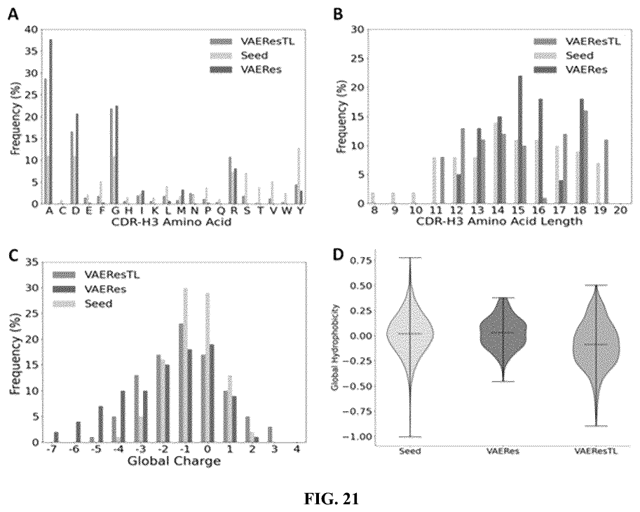

FIG. 21 shows a Visualize Comparison of molecular characteristics between Seed-sequences (Yellow), VAERes-generated sequences (Violet) and VAEResTL-generated sequences (Turquoise). Horizontal dashed lines account for the mean. Whiskers extend to the most extreme non-outlier data points. (A) amino acid distribution, (B) amino acid length distribution, (C) total charge distribution, (D) Eisenberg hydrophobicity.

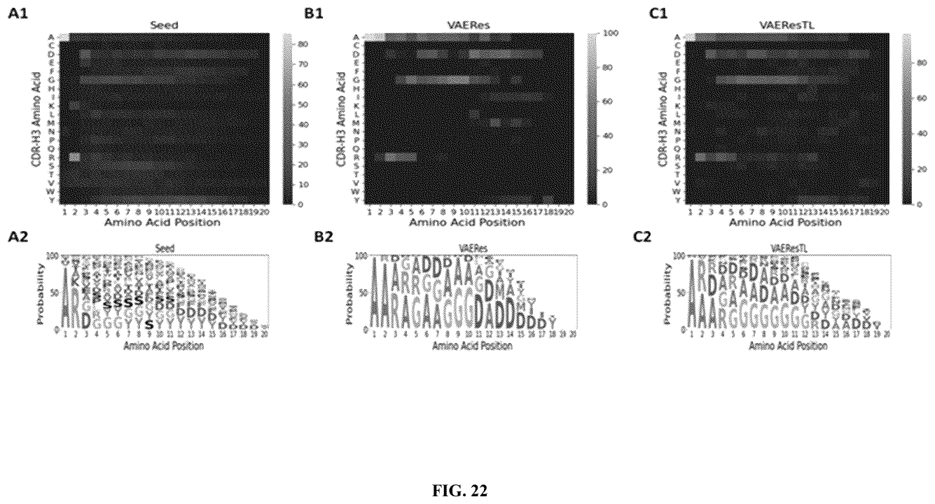

FIG. 22 shows a visualization of machine learning (ML). Heatmap visualization showing: (Al) the count of observed seed sequences. (B1) the count of VAERes-proposed sequences from (left) and to (right) each amino acid at each sequence position. (C1) the count of VAEResTL-proposed sequences from (left) and to (right) each amino acid at each sequence position. (Seed and VAERes-generated and VAEResTL-generated sequences have the length of 20). Sequence logo visualizations. (A2) Sequence logos for Seeds, (B2) VAERes-generated sequences (C2) VAEResTL-generated sequences fare based on residue frequency.

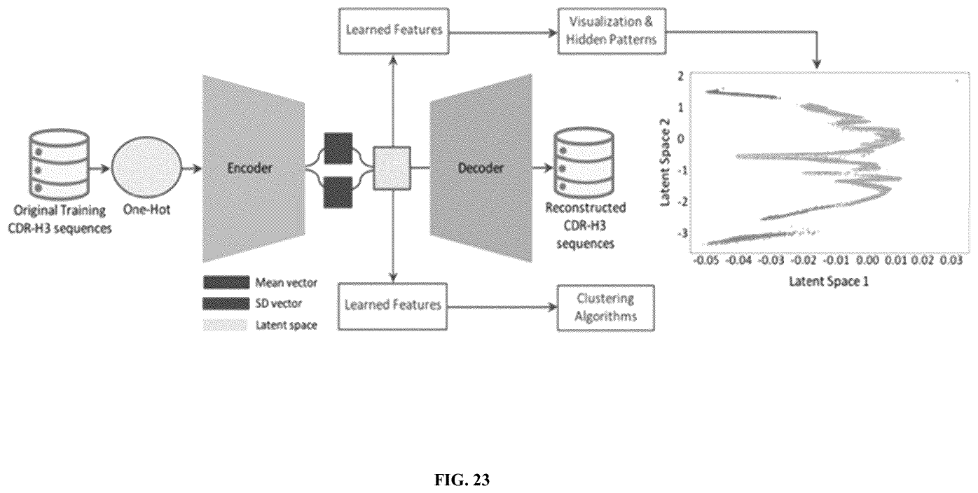

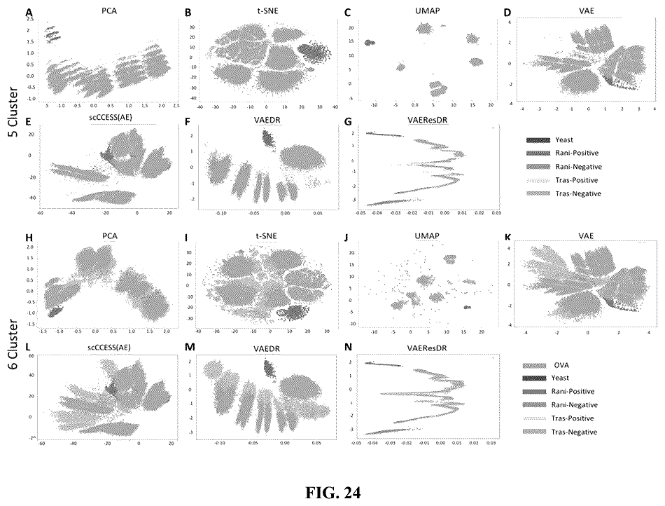

FIG. 23 illustrates an example of the model for the dimensionality reduction of antibody CDR-H3 sequences. A Resnet-adopted convolutional VAE was designed, referred to as VAEResDR. The VAEResDR comprises an encoder and a decoder. The encoder scales down high-dimensional input sequences into a two-dimensional feature space, while the decoder reconstructs the sequences from these two-dimensional features.

FIG. 24 shows a Visualization: 2-dimensional representation plots. Panel A to G representing PCA, t-SNE, UMPA, VAE, scCCESS (AE), VAEDR and VAEResDR for three targets, 5 clusters consecutively. Figure H to N representing PCA, t-SNE, UMPA, VAE, scCCESS (AE), VAEDR and VAEResDR for four targets, 6 clusters consecutively. OVA (orange dots), Yeast (red dots), Tars-Positive (yellow dots), Tras-Negative (turquoise dots), Rani-Positive (blue dots), and Rani-negative (green dots)).

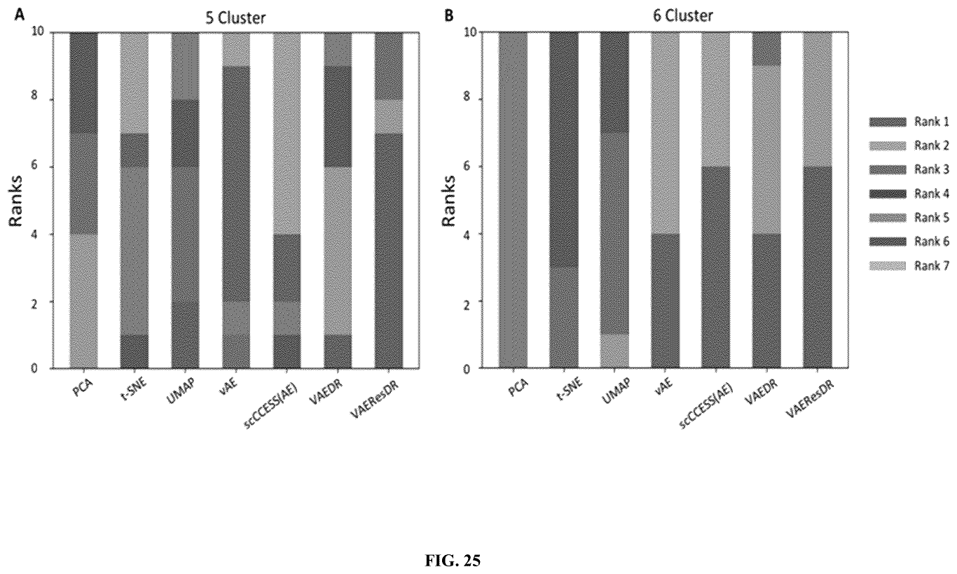

FIG. 25 shows a bar chart clustering comparison to demonstrate the DR methods' performance. The best performance is ranked 1 and the worst performance is ranked 7. The color codes for rank 1, 2, 3, 4, 5, 6, and 7 are blue, orange, green, red, purple, brown, and pink consecutively.

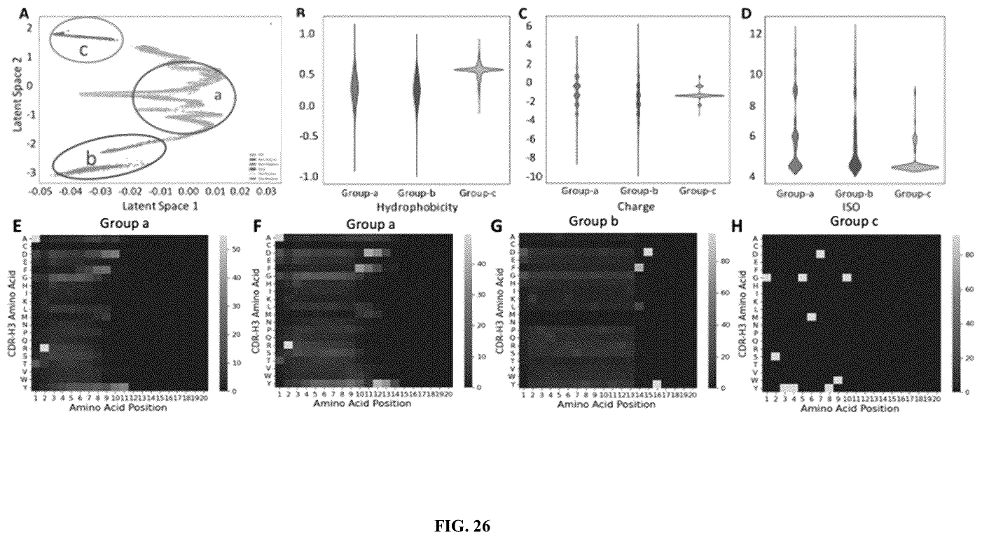

FIG. 26 shows a Hidden Patterns Analysis: Biophysical and ML visualization Heatmap analysis. (A) 2-dimensional visualization by VAEResDR method for four targets, 6 clusters. CDR-H3 Sequences sharing the same latent space are marked as group (a) and CDR-H3 sequences in different latent spaces are marked as group (b) and (c). (B) biophysical metrics of hydrophobicity (H) (C) net-charge, and (D) ISO. (E), (F), (G), and (H) are heatmap plots for group (a), (b), and (c) sequentially.

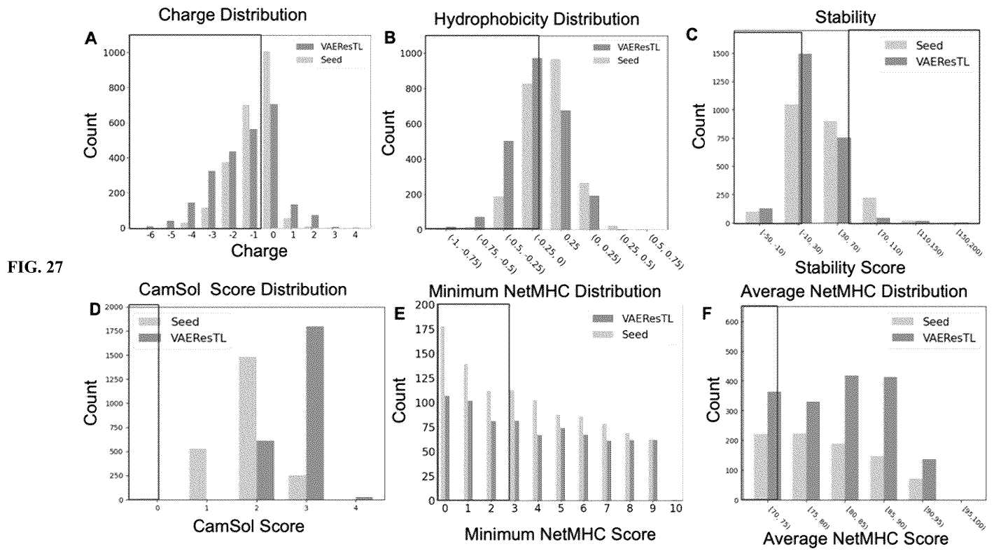

FIG. 27 shows a Visualize Comparison of molecular characteristics between Seed-sequences (Yellow), VAERes-generated sequences (Violet) and VAEResTL-generated sequences (Turquoise). Horizontal dashed lines account for the mean. Whiskers extend to the most extreme non-outlier data points. (A) amino acid distribution, (B) amino acid length distribution, (C) total charge distribution, (D) Eisenberg hydrophobicity.

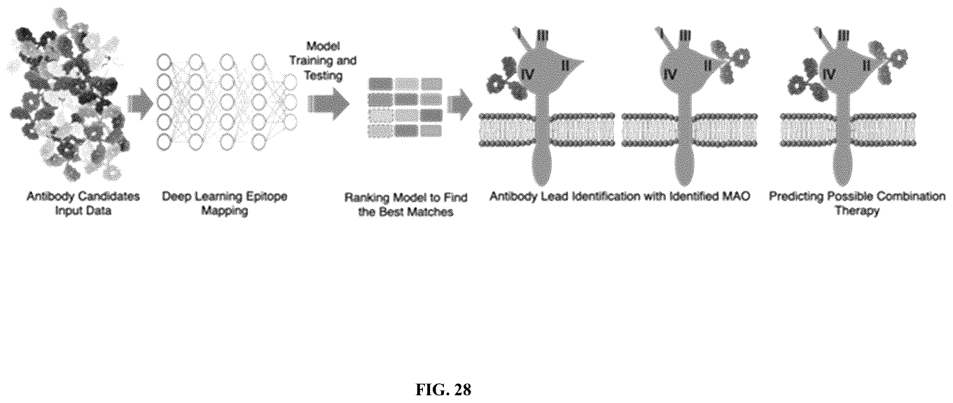

FIG. 28 illustrates an example of a Deep Learning Model that is developed and trained by antibody input data for Epitope Mapping. After the Deep Learning Model training and testing, a Ranking Model is used to analyze the antibody interactions with a given epitope to identify the best matches.

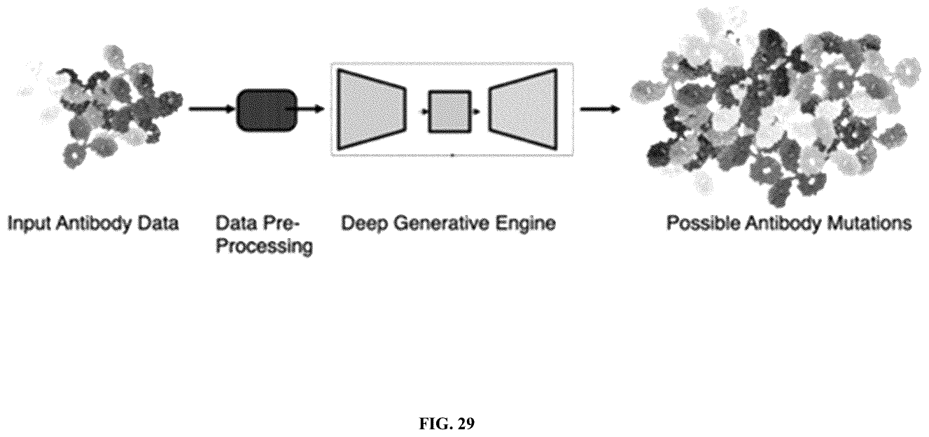

FIG. 29 illustrates an example illustration of a Deep Generative Engine. The VAEResTL, a deep generative model, is an end-to-end trainable model which comprises a convolutional Variational Autoencoder (VAE) that adopts a Resnet structure and is enhanced with a Transfer Learning (TL) technique. The method utilizes only the amino acid sequence data without the need for structural data and designs a large library of antibody mutations.

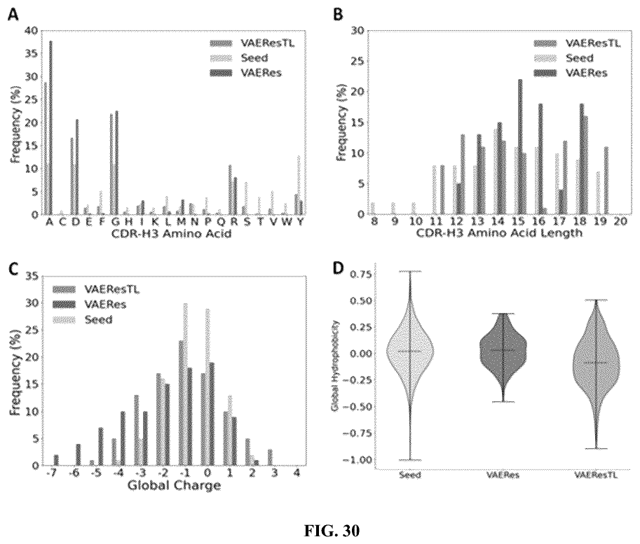

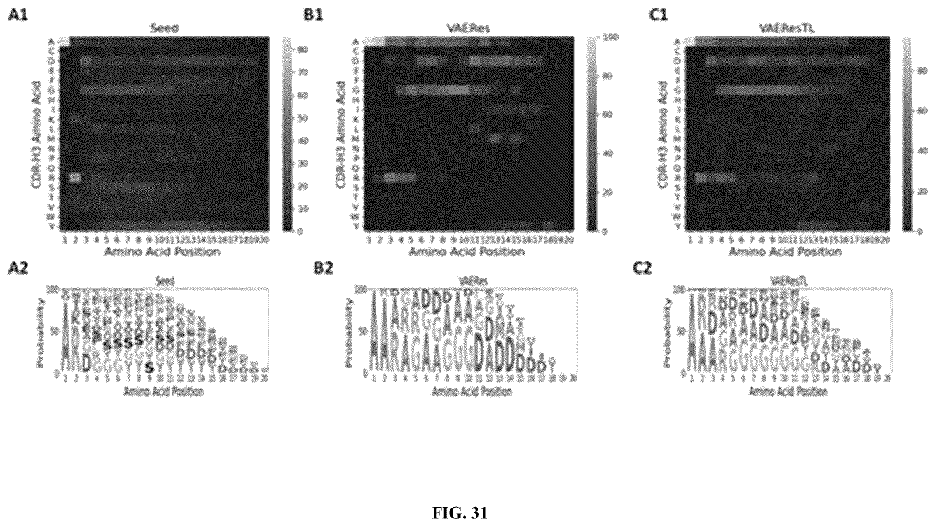

FIG. 30 shows a Visualize Comparison of molecular characteristics between Seed (SARS-CoV-2 seed sequences, Yellow), VAERes (VAERes-generated sequences for SARS-CoV-2, Violet), and VAEResTL (VAEResTL-generated sequences for SARS-CoV-2, Turquoise). Horizontal dashed lines account for the mean. Whiskers extend to the most extreme non-outlier data points. (A) amino acid distribution, (B) amino acid length distribution, (C) total charge distribution, and (D) Eisenberg hydrophobicity.

FIG. 31 shows a visualization of machine learning (ML). Heatmap visualization showing: (Al) the count of observed seed sequences. (B1) the count of VAERes-proposed sequences from (left) and to (right) each amino acid at each sequence position. (C1) the count of VAEResTL-proposed sequences from (left) and to (right) each amino acid at each sequence position. (Seed and VAERes-generated and VAEResTL-generated sequences have the length of 20). Sequence logo visualizations. (A2) Sequence logos for Seeds, (B2) VAERes-generated sequences (C2) VAEResTL-generated sequences fare based on residue frequency.

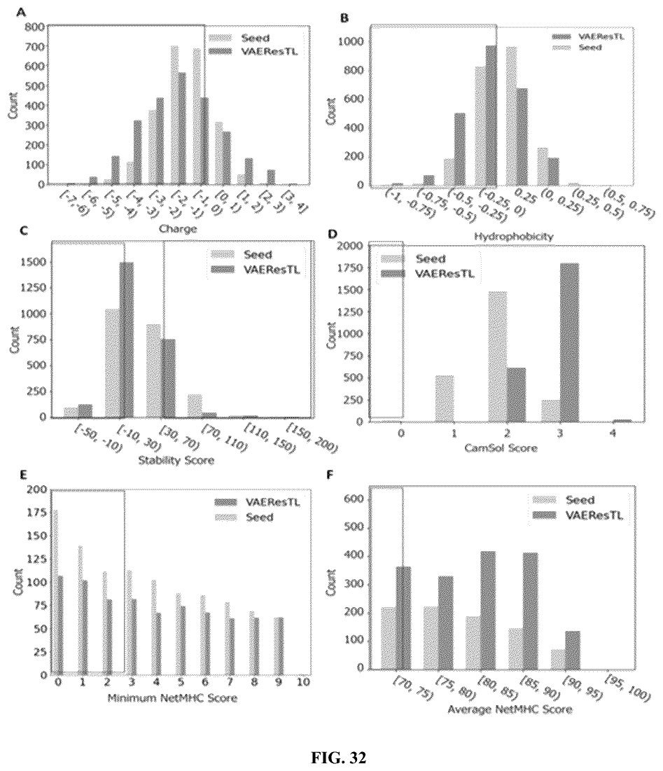

FIG. 32 shows an In-Silico Screening of predicted CDR-H3 sequences. Histograms present the parameter distributions of generated and seed SARS-CoV-2 sequences for different filtering steps. Boxes show the filtering cut-off for the therapeutic index in a clinical setting. (A) CDR-H3 net-charge. (B) CDR-H3 H. (C) CDR-H3 stability score. (D) CamSol solubility score. (E) the minimum NetMHCIIpan % Rank across all possible 15-mers for a given sequence and across all 26 HLA alleles. (F) the average NetMHCIIpan % Rank across all possible 15-mers for a given sequence and across all 26 HLA alleles.

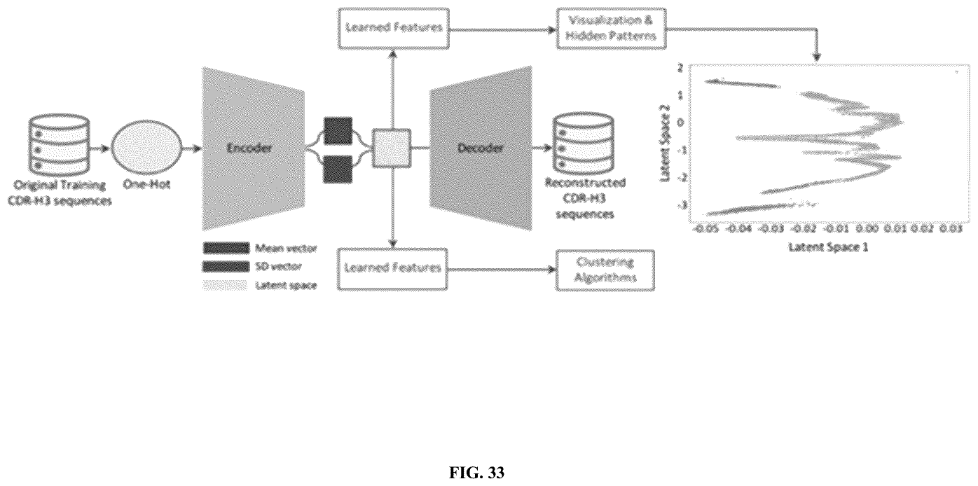

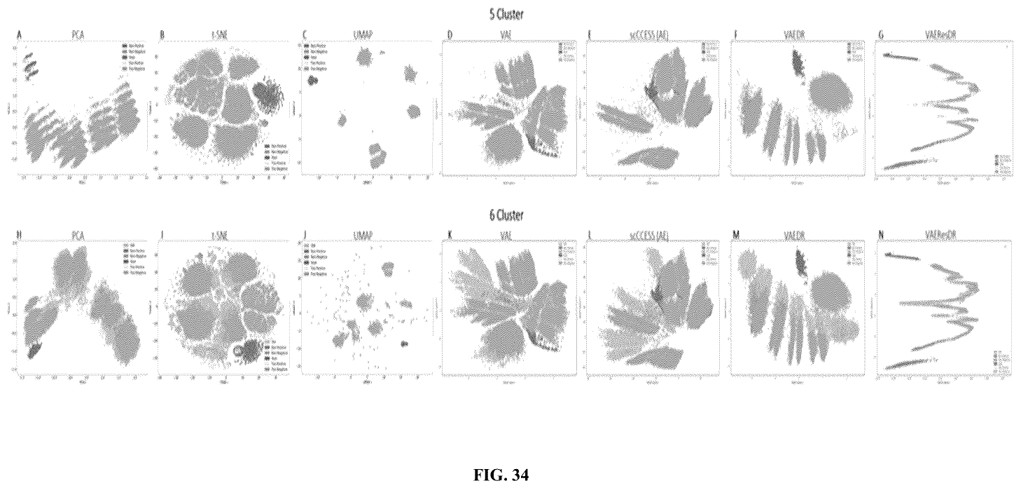

FIG. 33 shows a model for the dimensionality reduction of antibody CDR-H3 sequences. The VAEResDR is a Resnet-adopted convolutional VAE, which comprises an encoder and a decoder. The encoder scales down high-dimensional input sequences into a two-dimensional feature space, while the decoder reconstructs the sequences from these two-dimensional features.

FIG. 34 shows a Visualization of 2-dimensional representation plots, with panels A to G representing PCA, t-SNE, UMPA, VAE, scCCESS (AE), VAEDR, and VAEResDR for three targets, 5 clusters consecutively; and panels H to N representing PCA, t-SNE, UMPA, VAE, scCCESS (AE), VAEDR, and VAEResDR for four targets, 6 clusters consecutively. OVA (orange dots), Yeast display scFV (red dots), Tars-Positive (yellow dots), Tras-Negative (turquoise dots), Rani-Positive (blue dots), and Rani-negative (green dots)).

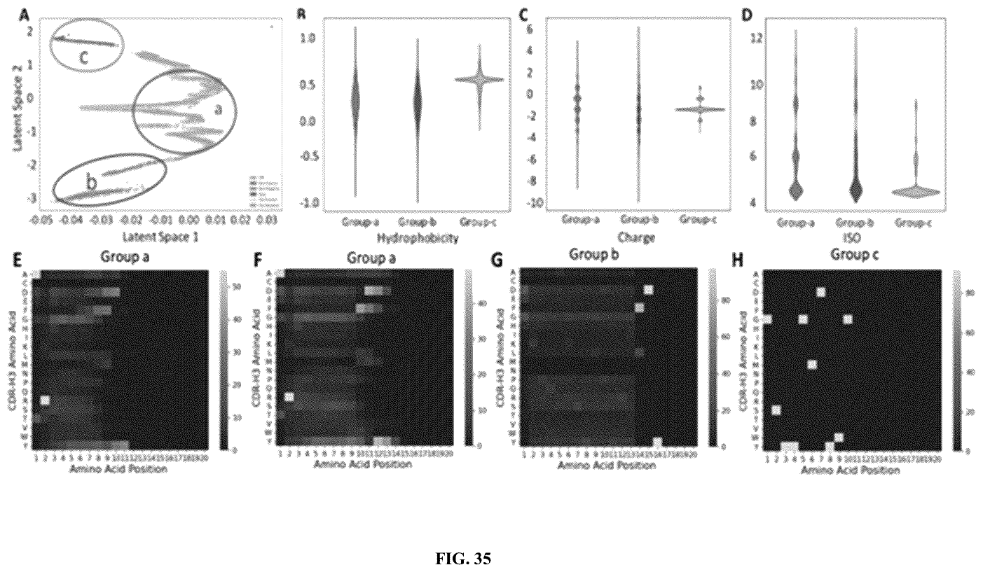

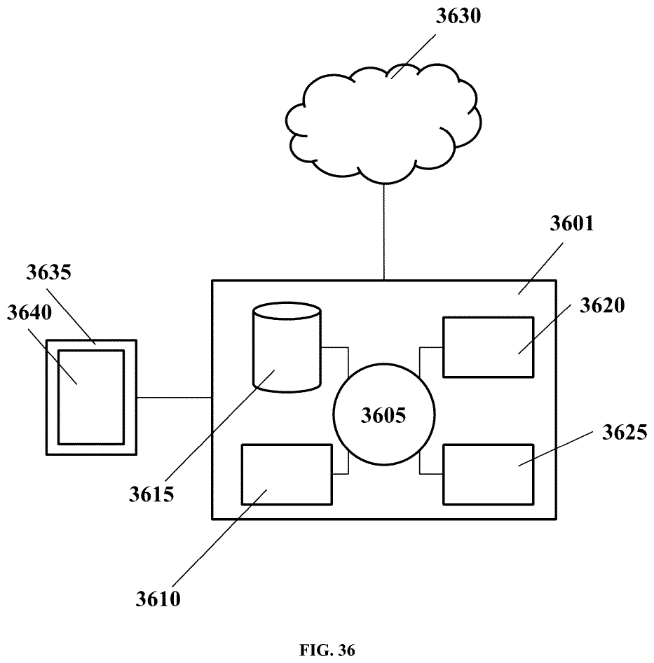

FIG. 35 shows a Hidden Patterns Analysis, including Biophysical and ML visualization Heatmap analysis. (A) 2-dimensional visualization by VAEResDR method for four targets, 6 clusters. CDR-H3 Sequences sharing the same latent space are marked as group (a) and CDR-H3 sequences in different latent spaces are marked as group (b) and (c). (B) biophysical metrics of hydrophobicity (H) (C) net-charge, and (D) ISO. (E), (F), and (G) are heatmap plots for group (a), (b), and (c) sequentially.

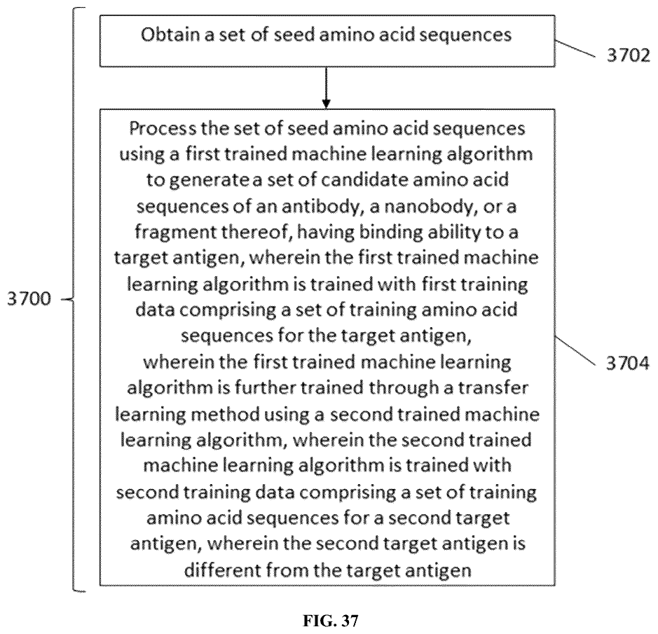

FIG. 37 shows an example method 3700 for generating a set of candidate amino acid sequences of an antibody, a nanobody, or a fragment thereof, having binding ability to a target antigen, using a transfer learning method. The method 3700 may comprise obtaining a set of seed amino acid sequences (as in operation 3702). The method 3700 may comprise processing the set of seed amino acid sequences using a first trained machine learning algorithm to generate a set of candidate amino acid sequences of an antibody, a nanobody, or a fragment thereof, having binding ability to a target antigen (as in operation 3704). The first trained machine learning algorithm may be trained with first training data comprising a set of training amino acid sequences for the target antigen. The first trained machine learning algorithm may be further trained through a transfer learning method using a second trained machine learning algorithm. The second trained machine learning algorithm may be trained with second training data comprising a set of training amino acid sequences for a second target antigen that is different from the target antigen.

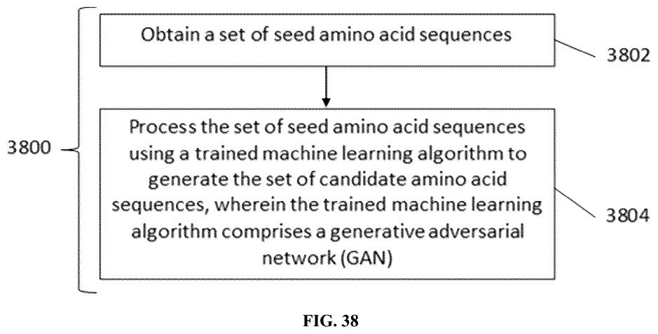

FIG. 38 shows an example method 3800 for generating a set of candidate amino acid sequences of an antibody, a nanobody, or a fragment thereof, having binding ability to a target antigen, using a generative adversarial network (GAN). The method 3800 may comprise obtaining a set of seed amino acid sequences (as in operation 3802). The method 3800 may comprise processing the set of seed amino acid sequences using a trained machine learning algorithm to generate the set of candidate amino acid sequences (as in operation 3804). The trained machine learning algorithm may comprise a generative adversarial network (GAN).

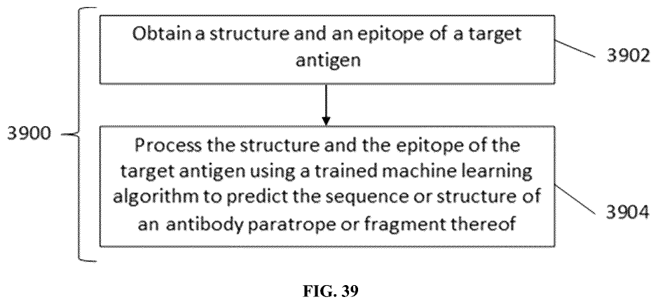

FIG. 39 shows an example method 3900 for predicting a sequence or structure of an antibody paratrope or fragment thereof, having binding ability to a target antigen. The method 3900 may comprise obtaining a structure and an epitope of a target antigen (as in operation 3902). The method 3900 may comprise processing the structure and the epitope of the target antigen using a trained machine learning algorithm to predict the sequence or structure of an antibody paratrope or fragment thereof (as in operation 3904).

According to some embodiments, at least one computer-readable storage medium size storing computer-executable instructions and/or a server storing and executing computer-executable instructions that, when executed by at least one processor can perform a model and/or method according to the techniques and methods described above.

The models may comprise deep learning-based models of large training datasets of antibody/nanobody amino acid sequences that are trained by using high performance graphic processing units (GPUs).

Described below are examples of ways in which the techniques and methods described above maybe applied in various embodiments. The below examples are merely for illustration purposes, and the embodiments are not limited to operating in accordance with any one or more of the examples. The below examples and the embodiments are not limited to operating in line with any one or more type of datasets and targets. Examples of models generated by machine learning based and deep learning-based methods using training data is discussed further below.

As described herein, the antibody/nanobody discovery techniques are implemented using deep learning models. Deep learning models include, but are not limited to, artificial neural networks.

An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can optionally be arranged in a plurality of layers such as input layer, output layer, and one or more hidden layers. Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tan H, or rectified linear unit (ReLU) function), and provide an output in accordance with the activation function. Additionally, each node is associated with a respective weight. ANNs are trained with a data set to minimize the cost function, which is a measure of the ANN's performance (e.g., error such as L1 or L2 loss) during training. The training algorithm tunes the node weights and/or bias to minimize the cost function. This disclosure contemplates that any algorithm that finds the minimum of the cost function can be used for training the ANN. Training algorithms for ANNs include, but are not limited to, backpropagation. An artificial neural network is provided only as an example machine learning model. This disclosure contemplates that the machine learning model may be a supervised learning model, semi-supervised learning model, or unsupervised learning model. The machine learning model may comprise a deep learning algorithm, an autoencoder (AE), a variational autoencoder (VAE), a support vector machine (SVM), a neural network, a Random Forest, a generative adversarial network (GAN), a graph neural network (GNN), a deep neural network (DNN), or a combination thereof.

### Computer Systems

The present disclosure provides computer systems that are programmed to implement methods of the disclosure. FIG. 32 shows a computer system 3201 that is programmed or otherwise configured to perform analyses of the methods, such as (i) generating a set of candidate amino acid sequences of an antibody, a nanobody, or a fragment thereof, having binding ability to a target antigen, (ii) predicting a sequence or structure of an humanized antibody paratrope or fragment thereof, having binding ability to a target antigen, (iii) predicting antibody paratope: antigen epitope interactions to design antibodies, nanobodies, or fragments thereof with identified mechanism of action, (iv) analyzing antibodies/nanobodies and/or fragments for their key biophysical and therapeutic properties, having the binding ability to a target antigen, (v) screening antibodies/nanobodies and/or fragments for their key biophysical and therapeutic properties through any automated and multimodal approaches, (vi) classifying antibodies/nanobodies and/or fragments having the binding ability to a target antigen, and (vii) repurposing antibodies/nanobodies and/or fragments having binding ability to a target, to other targets. The computer system 3201 can regulate various aspects of methods and systems of the present disclosure, such as, for example, perform an algorithm, input training data, analyze amino acid sequences, or output data. The computer system 3201 can be an electronic device of a user or a computer system that is remotely located with respect to the electronic device. The electronic device can be a mobile electronic device.

The computer system 3201 includes a central processing unit (CPU, also “processor” and “computer processor” herein) 3205, which can be a single core or multi core processor, or a plurality of processors for parallel processing. The computer system 3201 also includes memory or memory location 3210 (e.g., random-access memory, read-only memory, flash memory), electronic storage unit 3215 (e.g., hard disk), communication interface 3220 (e.g., network adapter) for communicating with one or more other systems, and peripheral devices 3225, such as cache, other memory, data storage and/or electronic display adapters. The memory 3210, storage unit 3215, interface 3220 and peripheral devices 3225 are in communication with the CPU 3205 through a communication bus (solid lines), such as a motherboard. The storage unit 3215 can be a data storage unit (or data repository) for storing data. The computer system 3201 can be operatively coupled to a computer network (“network”) 3230 with the aid of the communication interface 3220. The network 3230 can be the Internet, an internet and/or extranet, or an intranet and/or extranet that is in communication with the Internet. The network 3230 in some cases is a telecommunication and/or data network. The network 3230 can include one or more computer servers, which can enable distributed computing, such as cloud computing. The network 3230, in some cases with the aid of the computer system 3201, can implement a peer-to-peer network, which may enable devices coupled to the computer system 3201 to behave as a client or a server.

The CPU 3205 can execute a sequence of machine-readable instructions, which can be embodied in a program or software. The instructions may be stored in a memory location, such as the memory 3210. The instructions can be directed to the CPU 3205, which can subsequently program or otherwise configure the CPU 3205 to implement methods of the present disclosure. Examples of operations performed by the CPU 3205 can include fetch, decode, execute, and writeback.

The CPU 3205 can be part of a circuit, such as an integrated circuit. One or more other components of the system 3201 can be included in the circuit. In some cases, the circuit is an application specific integrated circuit (ASIC).

The storage unit 3215 can store files, such as drivers, libraries and saved programs. The storage unit 3215 can store user data, e.g., user preferences and user programs. The computer system 3201 in some cases can include one or more additional data storage units that are external to the computer system 3201, such as located on a remote server that is in communication with the computer system 3201 through an intranet or the Internet.

The computer system 3201 can communicate with one or more remote computer systems through the network 3230. For instance, the computer system 3201 can communicate with a remote computer system of a user (e.g., a medical professional or patient). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 3201 via the network 3230.

Methods as described herein can be implemented by way of machine (e.g., computer processor) executable code stored on an electronic storage location of the computer system 3201, such as, for example, on the memory 3210 or electronic storage unit 3215. The machine executable or machine readable code can be provided in the form of software. During use, the code can be executed by the processor 3205. In some cases, the code can be retrieved from the storage unit 3215 and stored on the memory 3210 for ready access by the processor 3205. In some situations, the electronic storage unit 3215 can be precluded, and machine-executable instructions are stored on memory 3210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 3201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 3201 can include or be in communication with an electronic display 3235 that comprises a user interface (UI) 3240 for providing, for example, an input of amino acid sequences, or an visual output relating to amino acid sequences or structure of an antibody/nanobody. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 3205. The algorithm can, for example, (i) generate a set of candidate amino acid sequences of an antibody, a nanobody, or a fragment thereof, having binding ability to a target antigen, (ii) predict a sequence or structure of an humanized antibody paratrope or fragment thereof, having binding ability to a target antigen, (iii) predict antibody paratope: antigen epitope interactions to design antibodies, nanobodies, or fragments thereof with identified mechanism of action, (iv) analyze antibodies/nanobodies and/or fragments for their key biophysical and therapeutic properties, having binding ability to a target antigen, (v) screen antibodies/nanobodies and/or fragments for their key biophysical and therapeutic properties through any automated and multimodal approaches, (vi) classify antibodies/nanobodies and/or fragments having the binding ability to a target antigen, and (vii) repurpose antibodies/nanobodies and/or fragments having binding ability to a target, to other targets.

### Example 1: Generative “VAEResTL” Model to Discover Amino Acid Sequences Against Novel Target Antigens

Antibodies play an important role in therapeutic discovery and vaccine development for a variety of diseases ranging from infectious diseases to cancer and autoimmune diseases (Zohar, T. and Alter, G. (2020). Dissecting antibody-mediated protection against sars-cov-2. Nature Reviews Immunology, 20(7):392-394, which is incorporated by reference herein). Wet-lab methods for antibody discovery may be very time-consuming and costly. One of these methods is high throughput screening, which is a drug discovery process used to identify the antibody leads that bind to their antigen targets and are within the therapeutics and developability index range (Sharma, V. K., Patapoff, T. W., Kabakoff, B., Pai, S., Hilario, E., Zhang, B., Li, C., Borisov, O., Kelley, R. F., Chorny, I., et al. (2014). In silico selection of therapeutic antibodies for development: viscosity, clearance, and chemical stability. Proceedings of the National Academy of Sciences, 111(52):18601-18606, which is incorporated by reference herein). The binding site of an antibody/nanobody includes a region referred to as complementarity determining region (CDR) (Murphy, K., Travers, P., Walport, M., and Janeway, C. (2008). Janeway's Immunobiology—7th (Seventh) edition. Garland Science, New York, which is incorporated by reference herein). Amongst CDRs, CDR-H3 on the heavy chain is the most variable CDR and may contribute the most to antigen specificity for antibodies and nanobodies (Tsuchiya, Y. and Mizuguchi, K. (2016). The diversity of h 3 loops determines the antigen-binding tendencies of antibody cdr loops. Protein Science, 25(4):815-825, which is incorporated by reference herein). The COVID-19 pandemic illustrates the importance of developing approaches capable of rapidly designing and developing therapeutics and prophylactics against novel viral infections. Designing CDR-H3 plays a critical role in antibody/nanobody-based therapeutics. Artificial intelligence (Al) and machine learning (ML) techniques may be used for COVID-19 vaccine development to stop the spread of the virus (Ong, E., Wong, M. U., Huffman, A., and He, Y. (2020). Covid-19 coronavirus vaccine design using reverse vaccinology and machine learning. Frontiers in immunology, 11:1581, which is incorporated by reference herein); however, rapid development of antibodies and nanobodies which can offer therapeutics and prophylactics benefits is of critical importance.

Biologically plausible deep learning (Yoo, D. K., Lee, S. R., Jung, Y., Han, H., Lee, H. K., Han, J., Kim, S., Chae, J., Ryu, T., and Chung, J. (2020). Machine learning-guided prediction of antigen-reactive in silico clonotypes based on changes in clonal abundance through bio-panning. Biomolecules, 10(3):421, which is incorporated by reference herein) and computational models (Adolf-Bryfogle, J., Kalyuzhniy, O., Kubitz, M., Weitzner, B. D., Hu, X., Adachi, Y., Schief, W. R., and Dun-brack Jr, R. L. (2018). Rosetta antibody design (rabd): A general framework for computational antibody de-sign. PLoS computational biology, 14(4):e1006112, which is incorporated by reference herein) may be applied to design and optimize CDR loops using deep sequencing data (Norman, R. A., Ambrosetti, F., Bonvin, A. M., Colwell, L. J., Kelm, S., Kumar, S., and Krawczyk, K. (2020). Computational approaches to therapeutic antibody de-sign: established methods and emerging trends. Brief-ings in bioinformatics, 21(5):1549-1567, which is incorporated by reference herein). Deep generative models using Variational Autoencoder (VAE) may be applied in designing proteins (Friedensohn, S., Neumeier, D., Khan, T. A., Csepregi, L., Parola, C., de Vries, A. R. G., Erlach, L., Mason, D. M., and Reddy, S. T. (2020). Convergent selection in antibody repertoires is revealed by deep learning. bioRxiv, which is incorporated by reference herein), and in predicting protein structures (Guo, X., Tadepalli, S., Zhao, L., and Shehu, A. (2020). Generating tertiary protein structures via an interpretative variational autoencoder. arXiv preprint arXiv:2004.07119, which is incorporated by reference herein). Moreover, deep Residual Neural Network models (Resnet) may also greatly improve protein design and protein structure predictions (Lu, S., Hong, Q., Wang, B., and Wang, H. (2020). Efficient resnet model to predict protein-protein interactions with gpu computing. IEEE Access, 8:127834-127844, which is incorporated by reference herein), and antibody-epitope classification (Ripoll, D. R., Chaudhury, S., and Wallqvist, A. (2021). Using the antibody-antigen binding interface to train image-based deep neural networks for antibody-epitope classification. PLoS computational biology, 17(3):e1008864, which is incorporated by reference herein). Nevertheless, there remains a need for application of Resnet in antibody/nanobody discovery. Deep learning and VAE-based approaches may be used in conjunction with large datasets and may be incapable of discovering and designing new antibodies/nanobodies when facing data shortage for novel targets such as SARS-CoV-2 and its variants. Transfer learning (TL) techniques may be used to perform in biomedical image classification and various protein prediction tasks (Heinzinger, M., Elnaggar, A., Wang, Y., Dallago, C., Nechaev, D., Matthes, F., and Rost, B. (2019). Modeling aspects of the language of life through transfer-learning protein sequences. BMC bioinformatics, 20(1):1-17; Valeri, J. A., Collins, K. M., Ramesh, P., Alcantar, M. A., Lepe, B. A., Lu, T. K., and Camacho, D. M. (2020). Sequence-to-function deep learning frameworks for engineered riboregulators. Nature communications, 11(1):1-14; each of which is incorporated by reference herein). However, there remains a need for incorporating a deep generative model of VAE with transfer learning (TL) to tackle the lack of training data in antibody/nanobody discovery. Using systems and methods of the present disclosure, deep learning algorithms are developed to predict therapeutic antibodies with binding ability to SARS-CoV-2. A novel end-to-end generative “VAEResTL” model is developed to discover amino acid sequences against novel target antigens that lack training data. The VAEResTL model is based on a VAE model, a Resnet structure, and a network-based TL technique. Resnet was demonstrated to improve the deep generative model VAERes's performance efficiently while providing a deep neural network capable of learning CDR-H3 sequence complexity. The learning from the VAERes pre-trained on sufficient antibody amino acid CDR-H3 sequences on different target antigens can be efficiently transferred for predicting antibody/nanobody amino acid sequences with binding ability to SARS-CoV-2. The VAEResTL predicted CDR-H3 sequences were then subject to in-silico screening and filtering for developability parameters namely viscosity, clearance, solubility, stability, and immunogenicity, resulting in potential lead antibody/nanobody CDR-H3 sequence candidates with optimal therapeutic properties.

As an example, a Variational Autoencoder (VAE) was developed, which adopts a Residual Neural Network (Resnet) structure to model and discover new CDR-H3 sequences for SARS-CoV-2. The VAE model further employs a transfer learning technique to address the data shortage problem for this novel target. The methodology is illustrated in FIG. 1.

Datasets were obtained and data pre-processing was performed as follows. Amino acid sequences of antibody CDR-H3 derived from deep sequencing data were used to develop and train the deep neural network algorithms. CDR-H3 sequences were filtered out based on their binding ability to SARS-CoV-2 (Raybould, M. I., Marks, C., Krawczyk, K., Taddese, B., Nowak, J., Lewis, A. P., Bujotzek, A., Shi, J., and Deane, C. M. (2019). Five computational developability guidelines for therapeutic antibody profiling. Proceedings of the National Academy of Sciences, 116(10):4025-4030, which is incorporated by reference herein), resulting in 2,298 sequences which were used as the primary training data. For the TL method, three large datasets were used on three different antigenic targets with a wide variety of antibodies, including ranibizumab (Rani) (The size of the dataset is 67,769) (Liu, G., Zeng, H., Mueller, J., Carter, B., Wang, Z., Schilz, J., Horny, G., Birnbaum, M. E., Ewert, S., and Gifford, D. K. (2020). Antibody complementarity determining region design using high-capacity machine learning. Bioinformatics, 36(7):2126-2133, which is incorporated by reference herein), yeast display scFV display scFv (Yeast display scFV) (The size of the dataset is 11,038) (Adams, R. M., Mora, T., Walczak, A. M., and Kinney, J. B. (2016). Measuring the sequence-affinity landscape of antibodies with massively parallel titration curves. Elife, 5:e23156, which is incorporated by reference herein), and chicken ovalbumin (OVA) (The size of the dataset is 65,638) (Goldstein, L. D., Chen, Y.-J. J., Wu, J., Chaudhuri, S., Hsiao, Y.-C., Schneider, K., Hoi, K. H., Lin, Z., Guerrero, S., Jaiswal, B. S., et al. (2019). Massively parallel single-cell b-cell receptor sequencing enables rapid discovery of diverse antigen-reactive antibodies. Communications biology, 2(1):1-10, which is incorporated by reference herein) to pre-train the algorithms.

Amino acid sequences were converted into a 2-dimensional matrix through one-hot encoding. To have the sequences of various lengths of 8-20 with the fixed lengths of 20, padding was performed, and a null character (e.g., “J”) was added to the left and right sides of sequences. Since there are 24 amino acids (20 standards, two rare (U, O), one unknown (X), and one null (J)), a CDR-H3 sequence with a length of 20 amino acids results in a 20×24 matrix. Each row includes of a single ‘1’ in the column corresponding to the amino acid in that position, whereby this value for all other columns in that row is equal to ‘0’.

Model architecture and training was performed as follows. The VAEResTL comprised an end-to-end trainable model with a convolutional VAE that adopts a Resnet structure and is enhanced with a TL technique. The method utilized only the amino acid sequences data without the need for structural data. CDR-H3 sequences may be subject to high variation in the amino acid distribution and may offer the highest contribution to antigen specificity. A deep neural network capable of learning from complex and highly variant sequences is essential to model CDR-H3 sequences; nevertheless, increasing the depth of the neural network by adding more layers leads to vanishing gradient problem (Goceri, E. (2019). Analysis of deep networks with residual blocks and different activation functions: classification of skin diseases. In 2019 Ninth international conference on image processing theory, tools and ap-plications (IPTA), pages 1-6. IEEE, which is incorporated by reference herein). Resnet introduces skip connections to jump over some layers. The skip connections allow gradients of a deep neural network to flow easily from layer to layer and prevent gradients from vanishing (He, K., Zhang, X., Ren, S., and Sun, J. (2016). Deep residual learning for image recognition. In Proceedings of the IEEE conference on computer vision and pattern recognition, pages 770-778, which is incorporated by reference herein). Resnet blocks were incorporated into the convolutional VAE model to increase the depth of the neural network for modeling and predicting CDR-H3 sequences, while preventing the vanishing gradient problem. The convolutional VAE that adopts a Resnet approach may be referred to as VAERes. Further, a network-based TL technique (Tan, C., Sun, F., Kong, T., Zhang, W., Yang, C., and Liu, C. (2018). A survey on deep transfer learning. In International conference on artificial neural networks, pages 270-279. Springer, which is incorporated by reference herein) was incorporated into the VAERes, which may be referred to as a VAEResTL model (FIG. 1).

Five experiments were executed and the results of VAE, VAERes, and VAEResTL were compared with baseline models of hidden Markov model (HMM) and long short-term memory (LSTM) neural network (Table 1A). In experiment 1 and 2, the convolutional VAE and VAERes were trained directly on the SARS-CoV-2 dataset. In experiment 3, (VAERes), the VAERes was trained directly on the SARS-CoV-2 dataset. In experiment 3, VAERes was trained on all the data together including Rani+Yeast display scFV+OVA sequences. Then the VAERes, which was pre-trained on Rani+Yeast display scFV+OVA sequences, was trained on SARS-CoV-2 dataset. In experiment 4 and 5, the baseline models of HMM and LSTM were trained directly on the SARS-CoV-2 dataset. HMM and LSTM are based on sequence models described as follows:
(a) LSTM: We developed an LSTM model, and replaced one-hot encoding with an embedding layer. The network includes two layers of LSTM with 100 units, cross-entropy loss function, and Adam optimizer.
(b) HMM: We developed an HMM model as a character-based model where amino acid characters are considered as states. Each state has a probability distribution over a set of possible sequences. Amino acid characters were then selected to form CDR-H3 sequences.

TABLE 1A
Baseline models comparison. Machine learning and biological
characteristics for CDR-H3 sequences generated for SARS-COV-2 by VAERes,
VAEResTL, HMM, and LSTM.

| | | | | | | Machine Learning Metrics | | | |
|---|---|---|---|---|---|---|---|---|---|
| | Biophysical Characteristics | | | | | BLEU | | | |
| Method | NW | H | ISO | Charge | Stability | (2-gram) | (3-gram) | (4-gram) | JSD |
| VAERes | 25.07 | 0.26 | 5.08 | −2.15 | 13.18 | 76.56 | 75.66 | 75.59 | 0.007 |
| VAEResTL- | 39.31 | 0.24 | 5.77 | −1.20 | 25.46 | 77.57 | 76.41 | 75.20 | 0.005 |
| ALL | | | | | | | | | |
| LSTM | 40.51 | 0.20 | 4.20 | −1.60 | 57.48 | 81.12 | 80.01 | 78.54 | 0.11 |
| HMM | 9.12 | 0.52 | 9.5 | 0.68 | 45.9 | 82.1 | 80.50 | 78.60 | 0.009 |
| Seed | NA | 0.23 | 5.64 | −0.84 | 33.36 | NA | NA | NA | NA |

TABLE 1B
Baseline models comparison: machine learning and biological
characteristics for CDR-H3 sequences generated for SARSCOV-2 by
VAEResTL-OVA, VAEResTL-Rani, and VAEResTL-Yeast display scFV.

| | Machine Learning Metrics | | | | | | | | |
|---|---|---|---|---|---|---|---|---|---|
| | BLEU | | | | Biophysical Characteristics | | | | |
| Method | (2-gram) | (3-gram) | (4-gram) | JSD | NW | H | ISO | Charge | Stability |
| VAEResTL- | 73.62 | 71.42 | 68.15 | 0.007 | 28.12 | 0.22 | 5.55 | −1.02 | 31.09 |
| OVA | | | | | | | | | |
| VAEResTL- | 79.59 | 78.75 | 77.90 | 0.006 | 41.05 | 0.23 | 6.18 | −0.76 | 29.08 |
| Rani | | | | | | | | | |
| VAEResTL- | 80.21 | 79.90 | 79.26 | 0.006 | 25.23 | 0.24 | 4.53 | −3.27 | 23.45 |
| Yeast | | | | | | | | | |

Machine learning (ML) and biophysical metrics were used to evaluate the methods' performance. In-silico screening was performed to assess the predicted antibody/nanobody CDR-H3 sequences that may bind to SARS-CoV-2.

Three different metrics were used to evaluate sequence similarity. First, Bilingual Evaluation Understudy (BLEU) (Papineni, K., Roukos, S., Ward, T., and Zhu, W.-J. (2002). Bleu: a method for automatic evaluation of machine translation. In Proceedings of the 40th annual meeting of the Association for Computational Linguistics, pages 311-318, which is incorporated by reference herein) was evaluated by employing 2-gram, 3-gram, and 4-gram to estimate BLEU, using the nltk python library. A higher BLEU score indicates a higher degree of similarity between the seed and the generated sequences. Second, statistical measures of Jensen-Shannon divergence (JSD) were used. When the JSD value is close to zero, the distribution of the generated sequences is very close to that of seed sequences. Third, pairwise sequence similarity method of Needleman-Wunch (NW), which is a biophysical characteristic, was used to evaluate the sequence similarity between seed and generated sequences (Wang, Y., Yadav, P., Magar, R., et al. (2020). Bio-informed protein sequence generation for multi-class virus mutation prediction. bioRxiv, which is incorporated by reference herein). The higher the NW value, the more similar the two sequences.

The sequence diversity was estimated by measuring the number of shared n-grams for different values of n between generated and seed sequences, referred to as Sn (Das, P., Wadhawan, K., Chang, O., Sercu, T., Santos, C. D., Riemer, M., Chenthamarakshan, V., Padhi, I., and Mojsilovic, A. (2018). Pepcvae: Semi-supervised targeted design of antimicrobial peptide sequences. arXiv preprint arXiv:1810.07743, which is incorporated by reference herein). Therefore, a value of Snmodel1/Snmodel2<1 implies more diversity of generated sequences by model 1 at a particular n compared to that of model 2.

Biophysical metrics were evaluated using the Bio and modLAMP library (Muller, A. T., Gabernet, G., Hiss, J. A., and Schneider, G. (2017). modlamp: Python for antimicrobial peptides. Bioinformatics, 33(17):2753-2755, which is incorporated by reference herein) which incorporates several modules, like descriptor calculation of biophysical characteristics of amino acid CDR-H3 sequences, e.g., stability, isoelectric-point, charge, and hydrophobicity (H) to evaluate the biophysical characteristics of predicted CDR-H3 sequences (Sharma, V. K., Patapoff, T. W., Kabakoff, B., Pai, S., Hilario, E., Zhang, B., Li, C., Borisov, O., Kelley, R. F., Chorny, I., et al. (2014). In silico selection of therapeutic antibodies for development: viscosity, clearance, and chemical stability. Proceedings of the National Academy of Sciences, 111(52):18601-18606, which is incorporated by reference herein).

In-silico screening was performed using the CamSol method at pH=7.0, a protein solubility predictor (Sormanni, P., Aprile, F. A., and Vendruscolo, M. (2015). The camsol method of rational design of protein mutants with enhanced solubility. Journal of molecular biology, 427(2):478-490, which is incorporated by reference herein), at pH=7.0, to estimate the protein solubility score for each CDR-H3 sequence. Each sequence variant's net-charge and hydrophobicity (H) were measured to predict the sequences' viscosity and clearance. We predicted the peptide binding affinity of the variant CDR-H3 sequences to MHC Class II molecules to a reference set of 26 HLA alleles by employing NetMHCIIpan (Jensen, K. K., Andreatta, M., Marcatili, P., Buus, S., Green-baum, J. A., Yan, Z., Sette, A., Peters, B., and Nielsen, M. (2018). Improved methods for predicting peptide binding affinity to mhc class ii molecules. Immunology, 154(3):394-406, which is incorporated by reference herein) to reduce their immunogenicity. The NetMHCIIpan's output provides a percentile rank that reflects sequences' affinity compared with a set of random natural peptides. The percentile rank classifies the peptides weak and strong binders to specific MHC Class II alleles. The strong binders have percentile rank above two, and the weak binders have percentile rank below ten. The minimum percentile rank is also classified as weak binders, with percentile rank below ten, and the average percentile rank is calculated across all 26 HLA alleles. The weaker the peptide affinity binding, the less immunogenic is a sequence (Mason, D. M., Friedensohn, S., Weber, C. R., Jordi, C., Wagner, B., Meng, S., Gainza, P., Correia, B. E., and Reddy, S. T. (2019). Deep learning enables therapeutic antibody optimization in mammalian cells by deciphering high-dimensional protein sequence space. BioRxiv, page 617860, which is incorporated by reference herein).

Comparison of the VAE models revealed that the convolutional VAE algorithm did not learn the complexity of CDR-H3 sequences, and 100% of the generated amino acid sequences were abnormal (invalid padding or invalid characters) and inappropriate for further analysis. Nevertheless, when the Resnet structure was incorporated into the convolutional VAE, the VAERes model demonstrated significant improvements in generating CDR-H3 sequences for SARS-CoV-2. When the VAERes was trained on SARS-CoV-2 seed CDR-H3 sequences, 98% of the generated sequences were valid (valid padding with valid characters), and only 2% of the generated sequences were abnormal (invalid padding or invalid characters). Further, 86.8% of the valid generated CDR-H3 sequences for SARS-CoV-2 were duplicate sequences, and only 11.2% of the generated sequences were unique. These results indicate that VAERes may learn a small range of CDRH3 sequences, reflecting the lack of adequate training data for SARS-CoV-2 seed CDR-H3 sequences as a new target. With VAEResTL, 100% of the generated CDR-H3 sequences for SARS-CoV-2 were valid (valid padding with valid characters), 70.7% of the valid generated sequences were unique, and only 29.3% of the CDR-H3 sequences were duplicate sequences.

Baseline models were compared using biophysical and NLP metrics. When HMM was trained on SARS-CoV-2 seed sequences, HMM was able to generate a very small library of CDR-H3 sequences which is 1/56 of the size of VAEResTL-generated library of SARS-CoV-2 CDRH3, where only 17% of the generated sequences were valid (valid padding with valid characters) and unique. These results indicate that HMM as a classic model may not be capable of learning the complexity of CDR-H3 sequences while suffering from lack of sufficient training data. LSTM was also able to generate a small library of sequences, where 95.5% of the sequences were valid (valid padding with valid characters). However, 86.1% of the valid generated CDR-H3 sequences for SARS-CoV-2 were duplicate sequences, and only 9.4% of the valid sequences were unique. LSTM was able to only generate a library of SARS-CoV-2 CDR-H3 sequences which is ⅙ of the size of VAEResTL-generated library of CDR-H3 sequences. A number of antibody heuristics were calculated, including Needleman (NW), Hydrophobicity (H), Isoelectric Point (ISO), Charge, and Stability, that give biological clues about how VAERes and VAEResTL perform compared to the baseline models of HMM and LSTM (Table 1A). The average pairwise sequence similarity of NW was consistently lower for VAERes, and HMM than VAEResTL. However, the higher NW for LSTM may be due to the bias of such a small ratio of the unique sequences among the small library of valid sequences. The average H value for VAEResTL generated sequences was closer to the seed sequences than generated sequences by VAERes, HMM, and LSTM. The ISO values ranged between 4.20 and 5.77 across all the other models that are close to the ISO values for the seed sequences, except the HMM generated sequences that has the highest ISO value. The average Charge values for all models were negative except for HMM. The average stability values for VAERes generated sequences were too low. The average stability values for LSTM generated sequences and HMM generated sequences were too high. Therefore, predicted sequences by VAERes, LSTM, and HMM were not appropriate for their biophysical stability property. Moreover, the VAEResTL had a stability value within the range of seed sequences. The overall BLEU values for VAEResTL generated sequences had higher values for 2-gram, 3-gram, and 4-gram than the BLEU for VAERes. The small ratio of valid and unique sequences for LSTM and HMM may bias their learning ability to only a small range of CDR-H3 sequences, reflecting the higher BLEU values for LSTM and HMM. Nonetheless, the JSD value for LSTM and HMM is higher than other models presented in Table 1A.

The overall ML and Biophysical metrics demonstrated that the VAEResTL model can more efficiently predict CDR-H3 sequences with binding ability to SARS-CoV-2. With transfer learning, VAEResTL is capable of learning a more “biologically plausible” latent space by utilizing a much larger dataset than VARes and the baseline models of LSTM and HMM. These results further indicated that VAEResTL architecture loads more biological context during the CDR-H3 sequence generation process. The baseline model comparison analysis indicated that VAEResTL outperforms baseline model techniques by predicting a more extensive library of valid and biologically more valuable antibody/nanobody CDR-H3 sequences with binding ability to SARS-CoV-2 more accurately despite the shortage of training data.

FIGS. 3A-3E show a comparison of predicted sequences by VAERes, VAEResTL, with the seed CDR-H3 sequences that bind to SARS-CoV-2 to visually imply the impact of transfer learning. Molecular features, e.g., sequence length, amino acid sequence distribution, charge, and H, were reported, as they play a crucial role in determining the membrane-binding affinity and specificity. The VAEResTL results were reported when the method is pre-trained by Rani+Yeast display scFV+OVA. The average amino acid composition (frequency and length distribution), net-charge, stability, and hydrophobicity (FIGS. 3A-3E) of generated CDR-H3 by VAEResTL matched the seed sequences more than the generated CDR-H3 by VAERes. This observation indicated that VAERes with TL performed well in capturing the charge patterning, hydrophobicity, and composition within generated sequences.

Further the diversity of the generated sequences was analyzed in terms of their shared n-grams (Sn). The n-gram similarity was lower for VAEResTL with respect to VAERes (SnVAEResTL/SnVAERes<1) for n>2 (2-gram, 3-gram, 4-gram, 5-gram are 0.91, 0.78, 0.63 and 0.44 respectively). These results indicated that VAEResTL sequences demonstrate strong long-range diversity; however, they are still consistent with biological sequences, as evident from sequence similarity comparison (Table 1A). The VAEResTL sequences showed more substantial diversity at higher n-grams (lower Sn values) as a desirable feature that can prevent viral resistance while designing next-generation antivirals and provide a more diverse pool of sequences for the in-silico screening and therapeutics development studies. Also, as indicated by the ML visualization heatmap (FIGS. 4A1-4C1) and logo plots (FIGS. 4A2-4C2) that, although VAEResTL changed certain CDR-H3 amino acid positions and their frequency distributions, the overall pattern of VAEResT-generated sequences were closer to seed sequences as compared to the VAERes-generated CDR-H3 sequences. High similarity of predicted CDR-H3 sequences and seed CDRH3 sequences observed by biophysical and ML analysis indicated that the VAEResTL-generated CDRH3 sequences also have binding ability to SARS-CoV-2. Moreover, ML and biophysical characteristics of the VAEResTL-generated CDR-H3 sequences (Table 1A, FIGS. 3A-3E, and FIGS. 4A1-C2) demonstrated that VAEResTL was able to predict more biologically valuable CDR-H3 sequences with binding ability to SARS-CoV-2, given that a diverse training database was used. These results show that the generalization for the VAEResTL in antibody/nanobody design is significantly improved.

With current advances in computational forecasts, a number of parameters including viscosity, clearance, solubility, stability, and immunogenicity are used as the guideline for in-silico screening to select the best in class lead candidates. The VAEResTL-generated SARS-CoV-2 CDR-H3 sequences were characterized compared to seed CDR-H3 sequences on a number of these in-silico methods. In order to screen the CDRH3 sequences' viscosity and clearance, net-charge and hydrophobicity (H) were determined by calculating every amino acid sequence of the CDR-H3 sequences. For all the sequences in the library, the net-charge was calculated at a given pH=7.0, and the hydrophobicity scale used was “eisenberg” (M Müller, A. T., Gabernet, G., Hiss, J. A., and Schneider, G. (2017). modlamp: Python for antimicrobial peptides. Bioinformatics, 33(17):2753-2755, which is incorporated by reference herein). The optimal net charge for drug clearance was between 0-6.2 with hydrophobicity of <0. Therefore, sequences with a net-charge of <0 (FIG. 5A, marked in box) and a H of <0 (FIG. 5B, marked in red box) were filtered out.

The stability of CDR-H3 sequences was further determined based on proteins and their dipeptide composition, and sequences with stability values of >40 and <20 (FIG. 5C, marked with red boxes) were filtered out. The VAEResTL-generated CDR-H3 amino acid sequences were analyzed using CamSol to estimate their solubility. Sequences with a CamSol score of <0:2 (FIG. 5D, marked with box) were filtered out. The low immunogenicity of antibodies/nanobodies may be an essential biophysical property for their therapeutics developability. The peptide binding affinity of all padded CDR-H3 sequences to MHC Class II was predicted using NetMHCIIpan to reduce their immunogenicity. We then used each peptide's % Rank of predicted affinity that we calculated when comparing CDR-H3 sequences with a set of 200,000 random natural peptides. The affinity for a set of 26 HLA alleles was predicted, which covers over 98% of the global population. The sequences with a % Rank of <2:5 (FIG. 5E, marked with box) (SARS-CoV-2 minimum % Rank) and % Rank of <70 (FIG. 5F, marked with box) (SARS-CoV-2 average % Rank) were filtered out. After employing in-silico screening antibody/nanobody CDR-H3 variants with desired viscosity, clearance, solubility, stability, and immunogenicity remained as potential lead candidates (Table 2). All remaining predicted CDR-H3 variants against SARS-CoV-2, marked outside the red boxes, confined values equal or superior to the parameters of the SARS-CoV-2 seed sequences. Through the in-silico screening, CDR-H3 sequence variants were identified with optimized multi-parameters that can be further evaluated in a wet-lab setting. Additional filters, including specificity and humanization, may be implemented to find the most developable therapeutic candidates. In addition, validation may be performed by mapping the predicted CDR-H3 sequences in-silico on protein/epitope targets.

TABLE 2
List of lead candidates amongst the CDR-H3 predicted
by VAEResTL after the in-silico screening with their
corresponding physiochemical properties such as net-
charge, hydrophobicity, solubility, and stability.

Predicted CDR-H3

| Sequences against | In-Silico screening values | | | |
|---|---|---|---|---|
| SARS-CoV-2 | Stability | Charge | Hydrophobicity | Solubility |
| ARARGGGGDAFAI | 31.18 | 0.80 | 0.09 | 1.95 |
| AAAGQFGGAGARRAF | 39.40 | 1.80 | 0.15 | 1.89 |
| ARAQSAAGAAANY | 36.5 | 0.80 | 0.06 | 1.82 |

A Resnet adapted VAE model was successfully developed for generating novel CDR-H3 sequences. Transfer learning techniques were used to maximize the power of the VAERes model for antibody/nanobody discovery when dealing with the lack of training data for novel targets such as SARS-CoV-2. The model was trained on hundreds of thousands of known and diverse CDR-H3 sequences from well-studied targets and created a readily usable tool with extensive generalization capabilities to discover new antibody/nanobody based therapeutics. To select antibodies/nanobodies with improved characteristics, the best lead CDR-H3 sequences were identified with binding ability to SARSCoV-2 through in-silico screening. These results may be used for validation of lead candidates through wet-lab experiments. Further, the model may be expanded for discovery of other CDR fractions to develop therapeutics against different variants of SARS-CoV-2 including Delta and Omicron, as well as other targets. The VAEResTL may be employed to design bispecific and trispecific antibodies to develop next generation disease therapeutics (e.g., cancer therapeutics).

### Example 2: VAEResDR Method for Dimensionality Reduction

Antibody-based therapeutics have increasingly fewer adverse effects due to their high specificity to given targets. Next-generation antibodies are offering promising classes of therapeutics for treating various diseases, particularly those requiring long-term treatment plans, e.g., cancer [14]. The third complementarity determining region of the heavy chains (CDR-H3) of an antibody/nanobody plays an essential role in recognizing and binding an antibody to its targets [26].

High variation in CDR-H3 amino acid sequences allows exploring antibody binding to one or multiple targets amongst a big pool of CDR-H3 sequences. However, this requires costly and laborious other wet-lab experimentation to screen and analyze CDR-H3 sequences for their specificity and binding responses to one or multiple targets [5]. In-silico analysis of antibody amino acid sequences can significantly shorten the time and reduce the cost of specificity screening and cross-reactivity studies [14]. Such analysis may also improve the utility of antibodies/nanobodies in multiple model organisms, offering potential therapeutic candidates for multiple targets [11].

Machine learning-based (ML) approaches such as clustering and dimensionality reduction (DR) may be developed to analyze the sequence databases. Clustering algorithms such as K-means [8], and Gaussian Mixture Model (GMM) [30] may be utilized for sequence analysis. Nonetheless, these algorithms work reasonably well for low-dimensional databases; however, for high-dimensional databases such as antibody sequences, their clustering performance degrades considerably due to the curse of dimensionality [17]. To reduce this computational complexity on large and high-dimensional antibody sequence databases, dimensionality reduction (DR) methods are used alongside clustering. Machine learning-based DR methods such as Principal Component Analysis (PCA) [29], t-distributed Stochastic Neighbor Embedding (t-SNE) [27], and Uniform Manifold Approximation and Projection (UMAP) [19] are utilized to reduce the sequences' dimensions. Yet, compared to original features, using clustering alongside these DR methods often degrades clustering quality since important features are lost during dimensionality reduction. Thus, a DR model capable of holding key features is required to obtain a more accurate and efficient sequence analysis.

Deep learning (DL) empowers finding the high-value bio-physicochemical parameters hidden within the sequences space resulting in improved sequence analysis and clustering due to their keen learning ability. DL methods have shown improvement in the clustering of bioimaging, biomedical text mining, and genomics sequences [10] due to their advanced featurization and DR ability. Antibody amino acid sequences analysis may be performed using ML [16, 13] and DL methods [22, 4]. Nevertheless, DL-based DR methods for multi-target specificity and cross-reactivity analysis of antibody sequences have not been explored yet.

Using systems and methods of the present disclosure, a deep learning model was developed to perform a dimensionality reduction method, and may be referred to as VAEResDR. The VAEResDR is based on a Resnet-adopted convolutional Variational Autoencoder (VAE). The scalable and generalizable VAEResDR was demonstrated to reduce sequences' high-dimensional space to a two-dimensional visualization space while keeping the important features. The key extracted features were utilized to accurately analyze antibody CDR-H3 sequences and effectively improve clustering. These analyses were indicative of whether CDR-H3 sequences have specificity or cross-reactivity to one or multiple targets.

Various architectures were developed to reduce the dimensions of amino acid sequences of CDR-H3 databases, group the amino acid sequences into clusters, and detect the hidden patterns within the amino acid sequences data. With unsupervised clustering approaches, CDR-H3 sequences that share similar, same, and/or different latent spaces within various clusters of CDR-H3 sequences were identified. These analyses were indicative of whether an antibody has multi-target specificity and/or cross-reactivity to one or multiple targets.

Four large datasets were utilized on four different antigenic targets with 183,284 CDR-H3 sequences as training data to train the algorithms. Trastuzumab (Tras) dataset contains 38,839 sequences, of which 29% are classified as strong binders (Tras-Positive), and 71% are classified as weak binders (Tras-Negative) [18]. The Ranibizumab (Rani) dataset contained 67,769 sequences, of which 22% are classified as strong binders (Rani-Positive), and 78% are classified as weak binders (Rani-Negative) [12]. The Yeast display scFV display scFv (Yeast display scFV) dataset contains 11,038 sequences [1]. Chicken Ovalbumin (OVA) contains 65,638 sequences [7].

A one-hot encoding method was used to convert amino acid sequences letters into an integer representation in a two-dimensional matrix. The amino acid CDR-H3 sequences' length varies between 8-20 among different databases. Therefore, Central padding was used to obtain sequences with fixed lengths of 20.

A Resnet-adopted convolutional VAE was developed, which may be referred to as VAEResDR. The VAEResDR architecture, as shown in FIG. 6, is based on the VAEResTL model, and comprises an encoder and a decoder. The encoder scales down high-dimensional input sequences into a two-dimensional feature space, while the decoder reconstructs the sequences from these two-dimensional features. To empower the encoder's ability to learn and extract the CDR-H3 sequences' key features more competently, an optimized number of Resnet blocks was incorporated into the encoder. Resnet blocks extend the depth of the neural network to expand the learning ability of the algorithm while preventing vanishing gradient problems. Moreover, to demonstrate the effectiveness of Resnet on improving the DR application of the model, the VAEResDR model (with Resnet blocks) was compared with a VAEDR model (a VAEResDR model without Resnet blocks). To train the VAEResDR model, all four datasets were used, containing 183,284 CDR-H3 sequences. The optimized VAEResDR successfully extracts key features as it scales down the CDR-H3 sequences into two-dimension spaces. The extracted key features were also fed into clustering algorithms to determine the similarity and diversity of CDR-H3 sequences and group them into different clusters, thereby resulting in improved clustering. The efficient feature extraction also enabled the mapping of CDR-H3 sequences better and the recognition of hidden patterns within the sequences space for a more efficient sequence analysis.

The VAEResDR model was optimized using the grid-search approach. The model is fed the input data while having optimal hyperparameters such as Adam optimizer, total loss=reconstruction loss (binary cross-entropy)+KL-Divergence loss, and a learning rate of 0.001.

In a first series of experiments, to assess the impact of the DL model in DR tasks, improved visualizations, and hidden patterns finding, three classic DR methods of PCA [29], t-SNE [27], UMAP [19], and two autoencoder-based models, VAE [2] and scCCESS (single-cell Consensus Clusters of Encoded Subspaces) models [6] were compared as baseline methods with the VAEDR and VAEResDR models. The Rani, Tras, and Yeast display scFV databases were used, in which the deep learning DR model was developed to visually recognize CDR-H3 sequences in 5 groups of Rani-Positive, Rani-Negative, Tras-Positive, Tras-Negative, and Yeast display scFV. The initial biophysical and ML analysis showed high similarity between OVA and Rani CDR-H3 sequences. Therefore, the OVA CDR-H3 sequences were included in a second series of experiments to illustrate the VAEResDR's performance in DR and biophysical feature extraction within the hidden patterns. In this experiment, the DR model was developed to visually illustrate CDR-H3 sequences in 6 groups of Rani-Positive, Rani-Negative, Tras-Positive, Tras-Negative, Yeast display scFV, and OVA and recognize whether there are CDR-H3 sequences with similar biophysical properties between different targets.

For benchmarking the DR performance and its effectiveness in clustering, two clustering algorithms of K-means [15] and Gaussian mixture model (GMM) [30] were used. The VAEResDR reported results obtained on a P100 GPU with 25 GB memory and a run time of 5 hours.

Visualization plots and clustering quality metrics were generated to evaluate the impact of the VAEResDR model on antibody amino acid CDR-H3 sequences' DR and clustering as compared to baseline methods. Further, biophysical metrics were measured and ML visualization heatmaps were generated to better demonstrate the biological validity of hidden patterns. The clustering quality metrics comprise five metrics: Mutual Information (MI) [25], Adjusted Rank Index (ARI) [9], Homogeneity Score (HS) [28], Completeness Score (CS) [28], and V-measure Score (VMS) [28]. The MI, ARI, HS, CS, and VMS values are confined in [0,1], equaling 1 when the two clusters are identical and 0 when they are independent. The larger values (closer to 1) are indicative of better performance. A set of biophysical metrics of Charge, Hydrophobicity (H), and Isoelectric Point (ISO) was used to screen CDR-H3 amino acid sequences, as they are highlighted to be the key features in antibody specificity. The bio and modLAMP libraries [20] were used to calculate the Charge, the H, and the ISO [24]. The Pairwise sequence similarity method of Needleman-Wunch (NW) was used to evaluate the similarity of sequences, where higher NW scores are indicative of more similarity between two sequences. The diversity of the sequences was determined by measuring the number of shared n-grams for different values of n between CDR-H3 sequences sharing the same latent space compared to CDR-H3 sequences in different latent spaces, referred to as Sn [3]. Therefore, a value of SnCondition1/SnCondition2<1 indicates more diversity of sequences in latent space Condition 1 at a particular n than Condition 2. We refer to Condition 1 for sequences in different latent spaces and Condition 2 for sequences in the same latent space.

The two-dimensional visualizations for five clusters in the series one experiments (FIGS. 7A-7G) and that of six clusters in the series two experiments (FIGS. 7A-7N) by the VAEResDR model are shown, as compared to the baseline models. In the series one experiments, the visualization plots demonstrate moderately weak separation in PCA (FIGS. 7A, FIG. 7H), t-SNE (FIG. 7B, FIG. 7I), UMAP (FIG. 7C, FIG. 7J), VAE (FIG. 7D, FIG. 7K), and scCCESS (AE) (FIG. 7E, FIG. 7L). Although the VAEDR model did recognize the three target datasets of Yeast display scFV, Tras, and Rani in different latent spaces, it was not able to recognize the two classes of Rani-Positive and Rani-Negative sequences and Tras-positive and Tras-Negative sequences (2F). In the series two experiments, when the OVA sequences were included in the input datasets, for ML methods of PCA, t-SNE, UMAP, and DL methods of VAE, and scCCESS (AE) (FIGS. 711-7L), OVA (orange dots) sequences spread in all latent spaces. However, although VAEDR (FIG. 7M) was able to well-separate the OVA sequences (orange dots) from Yeast display scFV and Tras sequences, it was unable to separate OVA sequences from Rani sequences. In VAEResDR visualization plots (FIG. 7G, FIG. 7N), moderately well-separated patterns were observed between 2 target datasets of Rani-Positive, Rani-Negative, and Yeast display scFV in both 5 clusters and 6 clusters except the Tras-Positive and Tras-Negative sequences. When including the OVA sequences in the input datasets, OVA (orange dots) sequences shared similar latent space with a portion of Rani-Negative and are well separated from Rani-Positive, Yeast display scFV, and Tras sequences (FIG. 7N). Comparing the VAEDR model's performance (without Resnet) with that of the VAEResDR model (Resnet), it was observed that Resnet improved the learning ability of the VAEResDR by separating the two classes of Rani-Positive and Rani-Negative, and was able to recognize the overlapping patterns of OVA with Rani-Negative sequences. With well-separated groups and valuable biophysical parameters recognized within the two-dimensional sequences space by the VAEResDR model, Tras-Positive and Tras-Negative sequences are not yet separated. These observations may be associated with the small range of weak and strong binders of Tras CDR-H3 sequences. The slight range difference between weak and strong binders may be due to the high accuracy of the CRISPR-Cas9 gene-editing method, a wet-lab antibody production technique used to produce Tras CDR-H3 sequences [18].

K-Means and GMM were tested on the extracted features by the VAEDR and VAEResDR models compared to the baseline models. Quality metrics of ARI, MI, HS, CS, and VMS were reported for five clusters (series one experiment) and six clusters (series two experiments) (Table 3), in which the VAEResDR model with GMM shows its best performance. In particular, according to the quantitative analysis, we observed MI of 0.915 and CS of 0.652 for five cluster experiments, and MI of 0.949 and CS 0.611 for six cluster experiments. When OVA sequences were added to the input datasets, the quantitative values for both K-Means and GMM degraded slightly due to the existing OVA sequences overlaps. Nevertheless, the VAEResDR outperformed PCA, t-SNE, UMAP, and DL methods of VAE, scCCESS (AE), and the VAEDR model (Table 3) in combination with clustering methods in both five cluster and six cluster experiments.

TABLE 3
Clustering baseline models comparison. Quality Metrics comparison of ARI, MI, HS, CS, VMS,
for K-Means and GMM on CDR-H3 sequences in 5 clusters and in 6 clusters.

| | Clustering | 5 Cluster | | | | | 6 Cluster | | | | |
|---|---|---|---|---|---|---|---|---|---|---|---|
| Method | Algorithms | ARI | MI | HS | CS | VMS | ARI | MI | HS | CS | VMS |
| PCA | K-Means | 0.316 | 0.7 [symbol] 2 | 0. [symbol] | 0. [symbol] 02 | 0. [symbol] 18 | 0.142 | 0.504 | 0.324 | 0.297 | 0.310 |
| | GMM | 0.344 | 0.786 | 0. [symbol] 0 | 0. [symbol] 21 | 0. [symbol] 0 | 0.124 | 0.48 [symbol] | 0.312 | 0.310 | 0.311 |
| t-SNE | K-Means | 0.258 | 0.602 | 0.429 | 0.380 | 0.4 [symbol] | 0.227 | 0.64 [symbol] | 0.363 | 0.363 | 0.388 |
| | GMM | 0.300 | 0.666 | 0.47 [symbol] | 0.422 | 0.447 | 0.239 | 0.685 | 0.441 | 0.384 | 0.413 |
| UMAP | K-Means | 0. [symbol] | 0.780 | 0. [symbol] | 0. [symbol] 01 | 0.527 | 0.200 | 0.634 | 0.408 | 0.366 | 0. [symbol] |
| | GMM | 0.342 | 0.774 | 0.552 | 0.498 | 0. [symbol] | 0.2 [symbol] | 0.767 | 0.494 | 0.442 | 0.467 |
| VAE | K-Means | 0.283 | 0.489 | 0.316 | 0.499 | 0. [symbol] 7 | 0.104 | 0. [symbol] | 0.255 | 0.273 | 0.263 |
| | GMM | 0.368 | 0.4 [symbol] 9 | 0. [symbol] | 0.478 | 0.39 [symbol] | 0.092 | 0.409 | 0.277 | 0.2 [symbol] 3 | 0.270 |
| scCCESS | K-Means | 0.2 [symbol] | 0.468 | 0.295 | 0.477 | 0.3 [symbol] | 0.128 | 0.4 [symbol] 8 | 0.261 | 0.295 | 0.277 |
| (AE) | GMM | 0.297 | 0.495 | 0.313 | 0. [symbol] | 0. [symbol] | 0.111 | 0.400 | 0.237 | 0.257 | 0.247 |
| VAEDR | K-Means | 0.449 | 0.681 | 0.448 | 0.695 | 0.541 | 0.328 | 0.840 | 0.484 | 0. [symbol] 1 | 0. [symbol] 11 |
| | GMM | 0.4 [symbol] 7 | 0.684 | 0.449 | 0.698 | 0.546 | 0. [symbol] 6 | 0.794 | 0.468 | 0.511 | 0. [symbol] |
| VAEResDR | K-Means | 0.4 [symbol] 7 | 0. [symbol] | 0.450 | 0.700 | 0.548 | 0. [symbol] 6 | 0. [symbol] | 0.48 [symbol] | 0.538 | 0.510 |
| | GMM | 0.530 | 0.915 | 0.683 | 0.652 | 0.667 | 0.426 | 0.949 | 0.552 | 0.613 | 0.580 |

[symbol]

indicates data missing or illegible when filed

To better illustrate the performance of DR methods in improving clustering analysis, the rank statistics of the compared methods were compared in a bar chart according to the quality metrics values (FIG. 8A, 5 clusters; and FIG. 8B, 6 clusters). The performance of the VAEResDR model ranked 1, 2, and 3 in 5 cluster experiments (FIG. 8A) and ranked 1 and 2 in 6 cluster experiments (FIG. 8B), demonstrating its best performance. The VALDR model achieved second place for its performance as it contains a distribution of ranks 1, 2, and 3 in 6 cluster experiments (FIG. 8B). The scCCESS (AL) and VAL showed the lowest performance, with a distribution performance rank of 6 and 7 in the 6 cluster experiments (FIG. 8B). The overall bar chart performance rank plots confirmed clustering performance degraded when OVA sequences were included in the input datasets. However, the VAEResDR model alongside GMM effectively showed high performance with improved antibody CDR-H3 sequences clustering. Other clustering approaches may be explored to determine the impact of the VAEResDR model in improving the clustering's performance.

Next, we quantitatively assessed whether the learned representation by the VAEResDR was able to prompt the biological characteristics of the CDR-H3 sequences on their specificity and cross-reactivity. The two-dimensional visualization plot provided by the VAResDR model was used for further analysis (FIG. 9A). CDR-H3 sequences were selected from different regions of the two-dimensional visualization plot, which were marked as group (a), group (b), and group (c) (FIG. 9A). CDR-H3 sequences in group (a) and group (b) have H (FIG. 9B), Charge (FIG. 9C), and ISO (FIG. 9D) values closer to each other that differentiate them from the group (c). The ML visualization heatmaps (FIGS. 9E-9G) also showed that sequences in group (a) and group (b) have higher similarity and the greatest difference with the sequences in the group (c). Further, the diversity of CDR-H3 sequences in the three mentioned groups were analyzed in terms of their shared n-grams (Sn). Sngroup(b&c)/Sngroup(a)<1, for n<2, 2-gram, 3-gram, 4-gram, and 5-gram are 0.76, 0.64, 0.54 and 0.25 respectively. These results were indicative of strong diversity between CDR-H3 sequences in group (a) with group (b) and (c). The average pairwise sequence similarity of NW for randomly selected sequences within group (a) was 44.2. The NW values for group (b) and group (c) were 32.5. These results demonstrated a higher biological similarity between sequences within group (a). The higher similarity in ISO, H, and Charge, which was confirmed by ML heatmap visualization plots for sequences in group (a) and group (b), demonstrated that the CDR-H3 sequences in group (a) and (b) may have specificity to multiple targets. Moreover, the higher NW score for sequences within group (a) demonstrated that the CDR-H3 sequences within group (a) may have specificity to multiple targets.

The color codes indicate group (a) is marked where OVA (FIG. 9A, orange dots) and Rani-Negative (FIG. 9A, green dots) are present. Group (b) is marked where Rani-Positive (FIG. 9A, blue dots) occupies the latent space and group (c) is marked where Yeast display scFV sequences (FIG. 9A, red dots) are accumulated. A close-up of the CDR-H3 sequences in group (a) illustrates that Rani-Negative CDR-H3 sequences share the same latent space with OVA CDR-H3 sequences. These results indicated that Rani-Negative sequences with weak binding to target Rani may have specificity to target OVA, and OVA sequences may have specificity to target Rani. The multi-target specificity of OVA sequences to Rani and OVA targets and Rani-Negative to OVA and Rani targets, may be used to expand the CDR-H3 sequences library for sequence screening in therapeutic developments of either targets. The sequence analysis by the VAEResDR model may also demonstrate that OVA sequences may have cross-reactivity with Rani target and Rani-Negative CDR-H3 sequences may have cross-reactivity with OVA target. With these analyses, false-positive or false-negative results may be prevented when performing sequence screening for either of the targets. The VAEResDR model may be used to expand the library of hit candidates to design the next generation of antibody-based therapeutics in-silico, especially for bispecific and trispecific antibody-based therapeutics.

A deep learning-based dimensionality reduction method, the VAEResDR model, was developed for antibody amino acid CDR-H3 sequence analysis. The quantitative analysis and results demonstrated that the VAEResDR model achieved superior DR performance in the two designed experimental series, and has significant generalizability for different datasets with diverse structure in the original and high-dimensional spaces. The VAEResDR model further demonstrated effective improvement in performing clustering of CDR-H3 sequences. This model provides a readily usable tool for screening of CDR-H3 sequences' specificity and for identifying highly specific CDR-H3 sequences that bind to one or multiple targets. The antibody analysis may be expanded to other antibody/nanobody sequences' fractions as well as other targets. The VAEResDR model may be applied to design bispecific and trispecific antibody therapeutics.
[1] Rhys M Adams, Thierry Mora, Aleksandra M Walczak, and Justin B Kinney. Measuring the sequence-affinity landscape of antibodies with massively parallel titration curves. Elife, 5:e23156, 2016, is incorporated by reference herein.
[2] Dongmei Ai, Yuduo Wang, Xiaoxin Li, and Hongfei Pan. Colorectal cancer prediction based on weighted gene co-expression network analysis and variational auto-encoder. Biomolecules, 10(9):1207, 2020, is incorporated by reference herein.
[3] Payel Das, Kahini Wadhawan, Oscar Chang, Tom Sercu, Cicero Dos Santos, Matthew Riemer, Vijil Chenthamarakshan, Inkit Padhi, and Aleksandra Mojsilovic. Pepcvae: Semi-supervised targeted design of antimicrobial peptide sequences. arXiv preprint arXiv:1810.07743, 2018, is incorporated by reference herein.
[4] Youyi Fong and Jun Xu. Forward stepwise deep autoencoder-based monotone nonlinear dimensionality reduction methods. Journal of Computational and Graphical Statistics, pages 1-10, 2021, is incorporated by reference herein.
[5] Norbert Furtmann, Marion Schneider, Nadja Spindler, Bjoern Steinmann, Ziyu Li, Ingo Focken, Joachim Meyer, Dilyana Dimova, Katja Kroll, Wulf Dirk Leuschner, et al. An end-to-end auto-mated platform process for high-throughput engineering of next-generation multi-specific antibody therapeutics. In Mabs, volume 13, page 1955433. Taylor & Francis, 2021, is incorporated by reference herein.
[6] Thomas A Geddes, Taiyun Kim, Lihao Nan, James G Burchfield, Jean YH Yang, Dacheng Tao, and Pengyi Yang. Autoencoder-based cluster ensembles for single-cell ma-seq data analysis. BMC bioinformatics, 20(19):1-11, 2019, is incorporated by reference herein.
[7] Leonard D Goldstein, Ying-Jiun J Chen, Jia Wu, Subhra Chaudhuri, Yi-Chun Hsiao, Kellen Schneider, Kam Hon Hoi, Zhonghua Lin, Steve Guerrero, Bijay S Jaiswal, et al. Massively parallel single-cell b-cell receptor sequencing enables rapid discovery of diverse antigen-reactive antibodies. Communications biology, 2(1):1-10, 2019, is incorporated by reference herein.
[8] John A Hartigan and Manchek A Wong. Algorithm as 136: A k-means clustering algorithm. Journal of the royal statistical society. series c (applied statistics), 28(1):100-108, 1979, is incorporated by reference herein.
[9] Lawrence Hubert and Phipps Arabie. Comparing partitions. Journal of classification, 2(1):193-218, 1985, is incorporated by reference herein.
[10] Md Rezaul Karim, Oya Beyan, Achille Zappa, Ivan G Costa, Dietrich Rebholz-Schuhmann, Michael Cochez, and Stefan Decker. Deep learning-based clustering approaches for bioinformatics. Briefings in Bioinformatics, 22(1):393-415, 2021, is incorporated by reference herein.
[11] David LaFleur, Donara Abramyan, Palanisamy Kanakaraj, Rodger Smith, Rutul Shah, Geping Wang, Xiao-Tao Yao, Spandana Kankanala, Ernest Boyd, Liubov Zaritskaya, et al. Monoclonal antibody therapeutics with up to five specificities: functional enhancement through fusion of target-specific peptides. In MAbs, volume 5, pages 208-218. Taylor & Francis, 2013, is incorporated by reference herein.
[12] Ge Liu, Haoyang Zeng, Jonas Mueller, Brandon Carter, Ziheng Wang, Jonas Schilz, Geraldine Horny, Michael E Birnbaum, Stefan Ewert, and David K Gifford. Antibody complementarity determining region design using high-capacity machine learning. Bioinformatics, 36(7):2126-2133, 2020, is incorporated by reference herein.
[13] Hao Lou and Michael J Hageman. Machine learning attempts for predicting human subcutaneous bioavailability of monoclonal antibodies. Pharmaceutical Research, 38(3):451-460, 2021, is incorporated by reference herein.
[14] Ruei-Min Lu, Yu-Chyi Hwang, I-Ju Liu, Chi-Chiu Lee, Han-Zen Tsai, Hsin-Jung Li, and Han-Chung Wu. Development of therapeutic antibodies for the treatment of diseases. Journal of biomedical science, 27(1):1-30, 2020, is incorporated by reference herein.
[15] James MacQueen et al. Some methods for classification and analysis of multivariate observations. In Proceedings of the fifth Berkeley symposium on mathematical statistics and probability, volume 1, pages 281-297. Oakland, CA, USA, 1967, is incorporated by reference herein.
[16] Rishikesh Magar, Prakarsh Yadav, and Amir Barati Farimani. Potential neutralizing antibodies discovered for novel corona virus using machine learning. Scientific reports, 11(1):1-11, 2021, is incorporated by reference herein.
[17] Rosalind B Marimont and Marvin B Shapiro. Nearest neighbour searches and the curse of dimensionality. IMA Journal of Applied Mathematics, 24(1):59-70, 1979, is incorporated by reference herein.
[18] Derek M Mason, Simon Friedensohn, Ce'dric R Weber, Christian Jordi, Bastian Wagner, Simon M Meng, Roy A Ehling, Lucia Bonati, Jan Dahinden, Pablo Gainza, et al. Optimization of therapeutic antibodies by predicting antigen specificity from antibody sequence via deep learning. Nature Biomedical Engineering, 5(6):600-612, 2021, is incorporated by reference herein.
[19] Leland McInnes, John Healy, and James Melville. Umap: uniform manifold approximation and projection for dimension reduction. 2020, is incorporated by reference herein.
[20] Alex T Mu*ller, Gisela Gabernet, Jan A Hiss, and Gisbert Schneider. modlamp: Python for antimicrobial peptides. Bioinformatics, 33(17):2753-2755, 2017, is incorporated by reference herein.
[21] Saul B Needleman and Christian D Wunsch. A general method applicable to the search for similarities in the amino acid sequence of two proteins. Journal of molecular biology, 48(3):443-453, 1970, is incorporated by reference herein.
[22] David Prihoda, Jad Maamary, Andrew Waight, Veronica Juan, Laurence Fayadat-Dilman, Daniel Svozil, and Danny Asher Bitton. Biophi: A platform for antibody design, humanization and humanness evaluation based on natural antibody repertoires and deep learning. bioRxiv, 2021, is incorporated by reference herein.
[23] Khalilian Saeed., Moti Zahra., Baloochestani Arian., Hallaj Yeganeh., Chavosh AliReza., and Hemmatian Zahra. Vaerestl: A novel generative model for designing complementarity determining region of antibody for sars-cov-2. In Proceedings of the 15th International Joint Conference on Biomedical Engineering Systems and Technologies—BIOINFORMATICS, pages 107-114. IN-STICC, SciTePress, 2022, is incorporated by reference herein.
[24] Vikas K Sharma, Thomas W Patapoff, Bruce Kabakoff, Satyan Pai, Eric Hilario, Boyan Zhang, Charlene Li, Oleg Borisov, Robert F Kelley, Ilya Chorny, et al. In silico selection of therapeutic antibodies for development: viscosity, clearance, and chemical stability. Proceedings of the National Academy of Sciences, 111(52):18601-18606, 2014, is incorporated by reference herein.
[25] Alexander Strehl and Joydeep Ghosh. Cluster ensembles-a knowledge reuse framework for combining multiple partitions. Journal of machine learning research, 3(Dec):583-617, 2002, is incorporated by reference herein.
[26] Yuko Tsuchiya and Kenji Mizuguchi. The diversity of h 3 loops determines the antigen-binding tendencies of antibody cdr loops. Protein Science, 25(4):815-825, 2016, is incorporated by reference herein.
[27] Laurens Van der Maaten and Geoffrey Hinton. Visualizing data using t-sne. Journal of machine learning research, 9(11), 2008, is incorporated by reference herein.
[28] Nguyen Xuan Vinh, Julien Epps, and James Bailey. Information theoretic measures for clusterings comparison: Variants, properties, normalization and correction for chance. The Journal of Machine Learning Research, 11:2837-2854, 2010, is incorporated by reference herein.
[29] Svante Wold, Kim Esbensen, and Paul Geladi. Principal component analysis. Chemometrics and intelligent laboratory systems, 2(1-3):37-52, 1987, is incorporated by reference herein.
[30] Zoran Zivkovic. Improved adaptive gaussian mixture model for background subtraction. In Proceedings of the 17th International Conference on Pattern Recognition, 2004. ICPR 2004., volume 2, pages 28-31. IEEE, 2004, is incorporated by reference herein.

### Example 3: Generative Adversarial Network for In-Silico Library Design of Antibody Variable Regions (AbGAN)

Provided herein are methods and systems for machine learning based design of antibodies and nanobodies in-silico. The methods and systems may comprise a Generative Adversarial Network for In-Silico Library Design of Antibody Variable Regions.

In-silico antibody library design methods may be developed to discover antibody (Ab) therapeutics. However, other methods may focus on the antibody's complementarity-determining regions (CDRs), specifically on the third CDR of the heavy chain (CDR-H3). While CDRs play a significant role in antibody binding and specificity, the antibody variable regions (Fvs) determine antibody binding, stability, and CDR orientation. Therefore, predicting antibody Fv sequences with improved therapeutics and developability properties may be of critical importance. Using systems and methods of the present disclosure, an end-to-end deep generative model was developed based on a generative adversarial network (GAN) and a Reinforcement Learning (RL) model, which may be referred to as AbGAN. The AbGAN model was demonstrated to be able to competently predict antibody Fv sequences, even in case with sparse training data. The results demonstrate that the AbGAN model outperforms other in-silico methods by generating a large and diverse library of more human-like antibody Fv sequences. Further, the method was demonstrated to be able to predict antibody Fv sequences of heavy chains (VH) and light chains (VL) to design monoclonal and bispecific antibody therapeutics. Through in-silico screening, the predicted antibody Fv sequences were demonstrated to present improved therapeutic and developability properties.

The next generation of antibody (Ab) therapeutics, including monoclonal, bispecific, and trispecific antibody therapeutics, are becoming an essential class of biologic-based therapeutics for known and novel diseases due to their unique specificity and efficacy. However, the long development times and high-cost are key challenges in the availability of antibody therapeutics (Xu et al., 2019) despite the increasing need to access those therapies. Moreover, given the massive antibody space, and the other methods' limitations, it is very likely that numerous antibody mutations are yet to be discovered for known or novel diseases.

To rapidly provide therapeutics for known or novel targets such as SARS-CoV-2, oncology targets (e.g., HER2), etc. to address the global pandemic, antibody therapeutics may need to be designed from a large and diverse landscape of antibody sequences and be optimized for activities within the therapeutics and developability indexes contemporary. In-silico antibody library design methods may be viable ways to boost the speed of antibody discovery while providing a massive antibody space. Such methods may comprise, for example, Generative Adversarial Networks (GANs), Autoencoder (AEs), and Variational Autoencoder Networks (VAEs), which may be statistically capable of generating unique sequences of proteins, nanobodies, and antibody complementary-determining regions (CDRs). Nevertheless, other in-silico library generation approaches may focus merely on designing the antibodies' complementary-determining regions (CDRs), specifically the CDRs in the heavy chains (CDR-H3). Despite the critical role of the CDRs in antibody therapeutics discovery due to their high specificity in binding to a target, other fractions of the variable region residues, including frameworks (FWRs), play a significant role in antibodies' therapeutic properties. Mutations in the FRs may lead to a shifted CDR which may impact antibody binding. Furthermore, FRs' mutations may lead to better binding by decreasing the entropy and antibody biochemical properties such as stability, charge, and hydrophobicity (Ewert et al., 2004). Thus, rapid design of the antibody's variable region, composed of seven amino acid regions, in which four are the frameworks (FWRs) and three are the complementarity determining regions (CDRs), may be of critical importance.

Using systems and methods of the present disclosure, the AbGAN model was developed as an in-silico approach to designing the next generation of antibody therapeutics. The AbGAN is a modified SeqGAN (Yu et al., 2017) for generating antibody variable regions (Fvs) in the heavy chain (VH). The AbGAN was able to learn natural antibody sequence diversity and generate functional antibody sequences to design monoclonal and bispecific antibody-based therapeutics. The generative method learns the transformative relationships of antibody sequences directly from the complex and high-dimensional amino acid sequences space and designs new, highly diverse sequence variants with improved physicochemical properties.

The AbGAN was demonstrated to be scalable and generalizable by generating antibody Fv sequences across a wide variety of antibodies, including SARS-CoV-2, human immunodeficiency virus (HIV), and chicken ovalbumin (OVA) as the case studies with the chemical and biophysical properties within the therapeutic index. The method was demonstrated to be able to generate a large and diverse library of antibody Fvs for heavy chains (VH) and light chains (VL). The AbGAN-generated FVs sequences were then subjected to in-silico screening for developability parameters such as clearance, viscosity, stability, solubility, immunogenicity, and humaneness. The in-silico library of FV sequences designed by the method was determined to have improved therapeutics and developability properties with more human-like properties.

Amino acid sequences of antibody variable regions from the heavy chains composed of three CDR and four framework (FR) fractions were derived from deep sequencing data and used to develop and train the method. Three datasets were utilized on three different antigenic targets containing human and non-human variable regions with a wide variety of antibodies, including SARS-CoV-2 (the size of the dataset is 5744 sequences) (Alsoussi et al., 2020), human immunodeficiency virus (HIV) (the size of the dataset is 5551 sequences) (Setliff et al., 2019), and chicken ovalbumin (OVA) (the size of the dataset is 65638 sequences) (Goldstein et al., 2019). The Keras tokenizer (Gulli & Pal, 2017) was used to convert the amino acid letters into integer representation. To have the sequences of the variable regions with various lengths of 107-134 with the fixed lengths of 134, we used a central padding technique (Liu et al., 2020).

The AbGAN is a generative model based on a generative adversarial network (GAN) and Reinforcement Learning (RL) architecturally tailored to learn long antibody sequences as discrete data. For the in-silico antibody library design, the method utilized only amino acid sequences of the variable region (Fv) without the need for structural data. The architecture of the AbGAN was based on the SeqGAN model (Yu et al., 2017), in which the generative model was combined with Reinforcement Learning (RL). The generative model considered a stochastic parametrized policy, in which a Monte Carlo search was used to estimate state and action value in RL. The generator took random noise to generate Fv sequences (FIG. 11). The discriminator was trained by the seed Fv sequences, and the Fv sequences were generated by the generator (FIG. 11). The generator was then trained by employing a policy gradient. The discriminator computed a defined reward and used it to determine the action values for the Monte Carlo search to predict the finished antibody Fv sequences ultimately. A different word embedding model called Global Vectors GloVe (Pennington et al., 2014) was implemented to the AbGAN, which does not rely only on local statistics but integrates global statistics to fetch word vectors. Although the AbGAN model architecture was inspired by the SeqGAN model, its hyperparameters were optimized by executing a randomized search of different variables, including nodes per layer, optimizer, loss function, activation function, and the number of the epoch.

To assess the generalizability of the AbGAN in generating antibodies with natural-like physicochemical properties, three experiments were executed, and the model was trained on a wide variety of antibody sequences. In experiment one, the AbGAN model was trained on SARS-CoV-2 Fv amino acid sequences. In experiment two, the AbGAN model was trained on HIV Fv amino acid sequences. In experiment three, the AbGAN model was trained on OVA Fv amino acid sequences. To further demonstrate the performance of the model, three experiments were executed, and the AbGAN model was compared with baseline models of LSTM (Gupta et al., 2018), VAEGP (Lim et al., 2018), and DCGAN (Radford et al., 2015) on antibody Fv sequences of SARS-CoV-2, HIV, and OVA.

Biophysical and machine learning (ML) metrics were generated and used to evaluate the performance of the model compared to baseline models. As a case study, the SARS-CoV-2 AbGAN-generated Fv sequences were further assessed across multiple biophysical properties through in-silico screening to identify new antibodies towards improved chemical and biophysical properties.

A number of biophysical metrics were generated and used, including isoelectric point (ISO), stability, charge, hydrophobicity (H), and aromaticity (Sharma et al., 2014), to demonstrate the similarity of amino acid sequences in the antibody variable regions. The Bio and modLAMP libraries (Muller et al., 2017) were used, which incorporates several modules, such as descriptor calculation of biophysical characteristics of amino acid sequences. Three machine learning metrics were generated and used to evaluate and compare the algorithms' performance. These metrics are Bilingual Evaluation Understudy (BLEU) (Papineni et al., 2002), Statistical measures of Jensen-Shannon divergence (JSD), and Maximum Mean Discrepancy (Lin, 1991).

To computationally assess the developability properties of the in-silico antibody Fv sequences generated by the Ab-GAN model, the sequences were screened on several key therapeutic properties. Each sequence variant's net-charge and hydrophobicity (H) (Sharma et al., 2014) were determined to predict the sequences' viscosity and clearance. The CamSol method (Sormanni et al., 2015) was used at pH=7.0 to estimate the protein solubility score for each Fv sequence.

To determine the scalability and generalizability of the AbGAN model for antibody in-silico library design, the model was trained on three different antigenic targets of SARS-CoV-2, HIV, and OVA. FIG. 12A compares the value of antibody heuristics, including Isoelectric Point (ISO) and Stability (S), for the in-silico generated Fv sequences for all three antigenic targets to evaluate the biophysical properties of Fv sequences compared to the seed sequences. The average biophysical characteristics of ISO and Stability for the AbGAN model-generated sequences were indicative of the high physicochemical similarity between generated Fv sequences and seed Fv sequences for all three antigenic targets of SARS-CoV-2, HIV, and OVA. The Machine Learning (ML) metrics of JSD and MMD values (FIG. 12A) across AbGAN-generated Fv sequences for three antigenic targets of SARS-CoV-2, HIV and, OVA were between 0.03-0.08. When the JSD and MMD values were close to zero, the distribution of the generated sequences and seed Fv sequences were very close. The UMAP visualization plot (FIG. 12B) further demonstrated that the AbGAN model was able to generate Fv sequences specific to their targets. In the UMAP plot, SARS-CoV2 seed Fv sequences (brown dots) and SARS-CoV2 AbGAN-generated Fv sequences (Blue dots) occupied similar latent space. The HIV seed Fv sequences (Torque dots) and HIV AbGAN-generated Fv sequences (Green dots) also occupied similar latent space, and were well separated from the Fv SARS-CoV-2 sequences. The OVA seed Fv sequences (Red dots) and OVA AbGAN-generated Fv sequences (Yellow dots) also occupied similar latent space, and were well separated from the SARS-CoV-2 and HIV. The UMAP visualization, biophysical characteristics, and ML metrics demonstrated that the AbGAN model was able to accurately generate Fv sequences with high specificity to their targets. These results show that the AbGAN model was able to learn a more biologically plausible latent space and offers generalizability properties in antibody design within a wide range of antigenic targets.

When the LSTM model was trained on the SARS-CoV-2, HIV, and OVA seed sequences, the LSTM model was able to generate an in-silico library of valid and unique Fv sequences as small as 1/29 of the size of AbGAN in-silico library of Fv sequences for SARS-CoV-2, 1/7 of the size of AbGAN in-silico library of Fv sequences for OVA and ¼ of the size of AbGAN in-silico library of Fv sequences for HIV. The DCGAN model also was able to generate a very small in-silico library of valid and unique Fv sequences as small as 1/28 of the size of AbGAN in-silico library of Fv sequences for SARS-CoV-2, 1/36 of the size of AbGAN in-silico library of Fv sequences for OVA, and 1/48 of the size of AbGAN in-silico library of Fv sequences for HIV. The VAEGP model, a VAE-based generator, was able to generate a large in-silico library of valid and unique Fv sequences as large as AbGAN. Further, the diversity of the generated Fv sequences was analyzed in terms of their shared n-grams (Sn) (Das et al., 2018). The n-gram similarity was lower for the AbGAN model with respect to the VAEGP model (Sₙ^{AbGAN}=Sₙ^{VAEGP}<1) for n>2. The 2-gram, 3-gram, 4-gram, 5-gram similarities were 0.97, 0.90, 0.88, and 0.81, respectively. These results indicated that the AbGAN-generated sequences showed strong long-range diversity. Despite the large in-silico library of Fv sequences generated by the VAEGP model, the VAEGP model exhibited lower diversity within the generated library of Fv sequences than the AbGAN model's in-silico library of Fv sequences. FIG. 13A compares the AbGAN, LSTM, VAEGP, and DCGAN models' predicted Fv sequences for SARS-CoV-2, HIV, and OVA with their seed Fv sequences for their biophysical characteristics. Molecular features, such as ISO, Hydrophobicity (H), Stability (S), aromaticity (A), and Charge (CH) were reported, as they play an essential role in defining antibody therapeutics' membrane binding affinity and specificity. To better demonstrate the performance of generative models compared to the model, in generating Fv sequences with improved biophysical properties, the statistics of the ranks of the compared methods were further reported in a bar chart according to the biophysical metrics values (FIG. 13B). The AbGAN model ranked 1 (color bar blue) and 2 (color bar orange) with a higher rank 1. The LSTM model ranked 2 (color bar orange), 3 (color bar green), and 4 (color bar red). The VAEGP model ranked 1 (color bar blue) and 2 (color bar orange) with a higher ratio of rank 2. The DCGAN model ranked 3 (color bar green) and 4 (color bar red). The overall bar chart performance rank plots also show the AbGAN model's performance is superior to that of other baseline models.

FIGS. 14A-14D compare SARS-CoV-2 generated Fv sequences by the AbGAN, LSTM, VAEGP, and DCGAN models with SARS-CoV-2 seed Fv sequences as a case example, and demonstrated the AbGAN model's performance compared to other baseline models visually. Molecular features, such as charge, amino acid sequence distribution, sequence length, and global hydrophobicity, were reported. The average net-charge (FIG. 14A), amino acid composition-frequency distribution (FIG. 14B), length distribution (FIG. 14C), and H (FIG. 14D) of the AbGAN-generated SARS-CoV-2 Fv sequences match the seed sequences more than the LSTM-generated, VAEGP-generated, and DCGAN-generated SARSCoV-2 Fv sequences. These results indicated that the AbGAN model performed well in learning the physicochemical properties of charge, H, and composition within seed Fv sequences to generate Fv sequences.

As a case example, all possible combinations of SARS-CoV-2 amino acids generated in-silico were used by the AbGAN model to validate the Fv sequences on their therapeutics parameters. Several physicochemical properties of amino acid sequences, e.g., viscosity, clearance, stability, and solubility, may preclude whether an antibody can become therapeutic. Although the generated sequences are not in the clinical stage yet, the AbGAN-generated SARS-CoV-2 Fv sequences were characterized and compared to seed SARS-CoV-2 Fv sequences through in-silico screening on these parameters. To screen the antibody variable region (Fv) viscosity and clearance, their net charge and hydrophobicity (H) were measured. For all the sequences in the in-silico library, the net charge was calculated at pH=7.0, and the sum across all residues of the variable regions was then calculated as the Fv net charge. The H values were also calculated and based on the “Eisenberg” values (Ewert et al., 2004).

The optimal net-charge for drug clearance may be between 0-6.2 with H of <0. Therefore, the sequences with a net-charge of <0 (FIG. 15A, marked with box) and an H of <0 (FIG. 15B, marked with box) were filtered out. The stability of variable region sequences was calculated based on proteins and their dipeptide composition with stability values of >40 and <20 (FIG. 15C, marked with boxes). The protein solubility score was determined for each full-length variable region sequence using the CamSol method (Sormanni et al., 2015) at pH 7.0. Sequences with a CamSol score of <0.2 (FIG. 15D, marked with box) were filtered out according to Sormanni et al. (Sormanni & Vendruscolo, 2019) guidelines. By the in-silico screening, Fv sequence variants were identified with optimized multi-parameters. However, additional filters, including immunogenicity, specificity, and humanization, may be implemented to find the most developable therapeutic candidates.

An end-to-end differentiable generative model, AbGAN, was developed, which is capable of synthesizing large and diverse sets of antibody Fv sequences with high fitness. The AbGAN model outperformed other in-silico methods by generating a large and diverse library of antibody Fv sequences. The antibody Fv sequences generated by AbGAN were demonstrated be usable to design monoclonal and bispecific antibodies. Further, the predicted antibody Fv sequences were demonstrated to present improved therapeutic and developability properties by utilizing an in-silico screening approach. These results may be used to develop human-like antibodies for different antigenic targets.

### REFERENCES

Alsoussi, W. B., Turner, J. S., Case, J. B., Zhao, H., Schmitz, A. J., Zhou, J. Q., Chen, R. E., Lei, T., Rizk, A. A., McIntire, K. M., et al. A potently neutralizing antibody protects mice against sars-cov-2 infection. The Journal of Immunology, 205(4):915-922, 2020 is incorporated by reference in its entirety.
Das, P., Wadhawan, K., Chang, O., Sercu, T., Santos, C. D., Riemer, M., Chenthamarakshan, V., Padhi, I., and Mojsilovic, A. Pepcvae: Semi-supervised targeted de-sign of antimicrobial peptide sequences. arXiv preprint arXiv:1810.07743, 2018 is incorporated by reference in its entirety.
Ewert, S., Honegger, A., and Plu ckthun, A. Stability improvement of antibodies for extracellular and intracellular applications: Cdr grafting to stable frameworks and structure-based framework engineering. Methods, 34(2): 184-199, 2004 is incorporated by reference in its entirety.
Friedensohn, S., Neumeier, D., Khan, T. A., Csepregi, L., Parola, C., de Vries, A. R. G., Erlach, L., Mason, D. M., and Reddy, S. T. Convergent selection in antibody reper-toires is revealed by deep learning. bioRxiv, 2020 is incorporated by reference in its entirety.
Goldstein, L. D., Chen, Y.-J. J., Wu, J., Chaudhuri, S., Hsiao, Y.-C., Schneider, K., Hoi, K. H., Lin, Z., Guerrero, S., Jaiswal, B. S., et al. Massively parallel single-cell b-cell receptor sequencing enables rapid discovery of diverse antigen-reactive antibodies. Communications biology, 2 (1):1-10, 2019 is incorporated by reference in its entirety.
Goodfellow, I., Pouget-Abadie, J., Mirza, M., Xu, B., Warde-Farley, D., Ozair, S., Courville, A., and Bengio, Y. Generative adversarial nets. Advances in neural information processing systems, 27, 2014 is incorporated by reference in its entirety.
Gulli, A. and Pal, S. Deep learning with Keras. Packt Publishing Ltd, 2017 is incorporated by reference in its entirety.
Gupta, A., Muller, A. T., Huisman, B. J., Fuchs, J. A., Schneider, P., and Schneider, G. Generative recurrent networks for de novo drug design. Molecular informatics, 37(1-2):1700111, 2018 is incorporated by reference in its entirety.
Lim, J., Ryu, S., Kim, J. W., and Kim, W. Y. Molecular generative model based on conditional variational autoencoder for de novo molecular design. Journal of cheminformatics, 10(1):1-9, 2018 is incorporated by reference in its entirety.
Lin, J. Divergence measures based on the shannon entropy. IEEE Transactions on Information theory, 37(1):145-151, 1991 is incorporated by reference in its entirety.
Liu, G., Zeng, H., Mueller, J., Carter, B., Wang, Z., Schilz, J., Horny, G., Birnbaum, M. E., Ewert, S., and Gifford, D. K. Antibody complementarity determining region design using high-capacity machine learning. Bioinformatics, 36(7):2126-2133, 2020 is incorporated by reference in its entirety.
Müller, A. T., Gabernet, G., Hiss, J. A., and Schneider, G. modlamp: Python for antimicrobial peptides. Bioinformatics, 33(17):2753-2755, 2017 is incorporated by reference in its entirety.
Papineni, K., Roukos, S., Ward, T., and Zhu, W.-J. Bleu: a method for automatic evaluation of machine translation. In Proceedings of the 40th annual meeting of the Association for Computational Linguistics, pp. 311-318, 2002 is incorporated by reference in its entirety.
Pennington, J., Socher, R., and Manning, C. D. Glove: Global vectors for word representation. In Proceedings of the 2014 conference on empirical methods in natural language processing (EMNLP), pp. 1532-1543, 2014 is incorporated by reference in its entirety.
Pinaya, W. H. L., Vieira, S., Garcia-Dias, R., and Mechelli, A. Autoencoders. In Machine learning, pp. 193-208. Elsevier, 2020 is incorporated by reference in its entirety.
Radford, A., Metz, L., and Chintala, S. Unsupervised representation learning with deep convolutional generative adversarial networks. arXiv preprint arXiv:1511.06434, 2015 is incorporated by reference in its entirety.
Repecka, D., Jauniskis, V., Karpus, L., Rembeza, E., Rokaitis, I., Zrimec, J., Poviloniene, S., Laurynenas, A., Viknander, S., Abuajwa, W., et al. Expanding functional protein sequence spaces using generative adversarial net-works. Nature Machine Intelligence, 3(4):324-333, 2021 is incorporated by reference in its entirety.
Riesselman, A., Shin, J.-E., Kollasch, A., McMahon, C., Simon, E., Sander, C., Manglik, A., Kruse, A., and Marks, D. Accelerating protein design using autoregressive gen-erative models. BioRxiv, pp. 757252, 2019 is incorporated by reference in its entirety.
Setliff, I., Shiakolas, A. R., Pilewski, K. A., Murji, A. A., Mapengo, R. E., Janowska, K., Richardson, S., Oosthuy-sen, C., Raju, N., Ronsard, L., et al. High-throughput mapping of b cell receptor sequences to antigen speci-ficity. Cell, 179(7):1636-1646, 2019 is incorporated by reference in its entirety.
Sharma, V. K., Patapoff, T. W., Kabakoff, B., Pai, S., Hilario, E., Zhang, B., Li, C., Borisov, O., Kelley, R. F., Chorny, I., et al. In silico selection of therapeutic antibodies for development: viscosity, clearance, and chemical stability. Proceedings of the National Academy of Sciences, 111 (52):18601-18606, 2014 is incorporated by reference in its entirety.
Sormanni, P. and Vendruscolo, M. Protein solubility pre-dictions using the camsol method in the study of protein homeostasis. Cold Spring Harbor perspectives in biology, 11(12):a033845, 2019 is incorporated by reference in its entirety.
Sormanni, P., Aprile, F. A., and Vendruscolo, M. The cam-sol method of rational design of protein mutants with enhanced solubility. Journal of molecular biology, 427 (2):478-490, 2015 is incorporated by reference in its entirety.
Xu, Y., Wang, D., Mason, B., Rossomando, T., Li, N., Liu, D., Cheung, J. K., Xu, W., Raghava, S., Katiyar, A., et al. Structure, heterogeneity and developability assessment of therapeutic antibodies. In MAbs, volume 11, pp. 239-264. Taylor & Francis, 2019 is incorporated by reference in its entirety.
Yu, L., Zhang, W., Wang, J., and Yu, Y. Seqgan: Sequence generative adversarial nets with policy gradient. In Proceedings of the AAAI conference on artificial intelligence, volume 31, 2017 is incorporated by reference in its entirety.

### Example 4: Improving Antibody Sequences Classification using BioOne-Hot Preprocessing

Antibody therapeutics have become increasingly popular drugs against various targets specifically where small molecule drugs and other therapeutics have proven to be inefficient. Antibody therapeutics often require binding to a specific epitope in an antigenic target to employ their functions. These functions may include e.g., blocking a ligand-receptor interaction, drawing out agonistic activities on receptors, and engaging two proteins to form a complex. Therefore, it may be essential to select and identify antibodies that bind to a specific conformational epitope of an antigen when developing antibody therapeutics. Machine learning (ML)-based classification methods may be applied in antibody drug design to select the antibody lead candidates. ML classification methods identify antibodies that bind to a specific antigenic target. However, ML classification methods may lack sufficient accuracy and performance in antibody classification due to the complexity of antibody sequences. Therefore, systems and methods of the present disclosure may comprise performing a data pre-processing method using amino acids' bio-physicochemical properties to classify the antibodies that are specific to a target more accurately. The data pre-processing approach was evaluated for the classification of antibodies with different binding strengths to HER2, VEGF-A, and MHC II receptors.

A data preprocessing approach was developed based on the One-Hot encoding to convert the amino acid sequences of CDR-H3 into integers for improving antibody sequences classification. With the preprocessing approach, CDR-H3 sequences specific to different targets were grouped, and the CDR-H3 sequences were classified into groups with different binding strengths to a specific target. These analyses were used to identify antibody lead candidates based on their therapeutic properties and enhance the antibody therapeutics design process.

Four large datasets were utilized on three different antigenic targets with 172,246 sequences as training data to train the algorithms. The antibody sequences datasets comprised the antibody third complementarity determining region of the heavy chain (CDR-H3) sequences. The Trastuzumab (Tras) dataset contained 38,839 sequences, of which 29% were classified as strong binders (Tras-Positive), and 71% were classified as weak binders (Tras-Negative) [1]. The Ranibizumab (Rani) dataset contained 67,769 sequences, of which 22% were classified as strong binders (Rani-Positive), and 78% were classified as weak binders (Rani-Negative) [2]. The Chicken Ovalbumin (OVA) contains 65,638 sequences [3].

Amino acid sequences were converted into a 2-dimensional matrix by using a modified version of the one-hot encoding process. Amino acids' bio-physicochemical values were incorporated into the One-Hot encoding, which may be referred to as BioOne-Hot encoding. In the BioOne-Hot encoding, each row includes a single ‘value’ or a ‘combination of several values’ associated with amino acids' bio-physicochemical properties in the column corresponding to that amino acid in that position, whereby this value for all other columns in that row is equal to ‘0’. The bio-physicochemical properties comprise isoelectric point, volume, hydrophobicity, solubility, charge, and solvent-accessible surface area (SASA). The antibody CDR-H3 sequences have various lengths; hence, they may not be suitable for use as input data of a machine learning model directly. Thus, three types of padding methods were used, Pre-Padding, Post-Padding, and Ext-Padding to evaluate which method best fixed the length of CDRH3 amino acid sequences to 20 across all antibodies CDR-H3 sequences. Since there are 24 amino acids (20 standards, two rare (U, O), one unknown (X), and one null (J)), a CDR-H3 sequence with a length of 20 amino acids resulted in a 20×24 matrix. Regular One-Hot encoding was used as a baseline model, in which, each row includes a single ‘1’ in the column corresponding to the amino acid in that position, whereby this value for all other columns in that row is equal to ‘0’.

For benchmarking the impact of BioOne-Hot encoding in improving the antibody amino acid sequences' classification, four machine learning-based classification methods were used: Decision Tree (DT), K-Nearest Neighbors (KNN), Support-Vector Machines (SVM), and Random Forests (RF).

Three experiments were executed, and each of the classifiers was compared with implemented BioOne-Hot encoding as compared to the classifiers with implemented One-Hot encoding. In experiment 1, Tras-Positive and Tras-Negative were used as the input data, and BioOne-Hot encoding was used with implemented amino acid bio-physicochemical properties to convert the sequences to integers. The DT, KNN, SVM, and RF classifiers were trained using BioOne-Hot preprocessed sequences of Tras-Positive and Tras-Negative. The classifiers were train to distinguish the sequences into two classes.

In experiment 2, Tras-Positive, Tras-Negative, Rani-Positive, and Rani-Negative were used as the input data, and BioOne-Hot encoding was used with implemented amino acid bio-physicochemical properties to convert the sequences to integers. The DT, KNN, SVM, and RF classifiers were used using BioOne-Hot preprocessed sequences of Tras-Positive, Tras-Negative, Rani-Positive, and Rani-Negative. The classifiers were trained to distinguish the sequences into four classes.

In experiment 3, OVA, Tras-Positive, Tras-Negative, Rani-Positive, and Rani-Negative were used as the input data and used BioOne-Hot encoding with implemented amino acid bio-physicochemical properties to convert the sequences to integers. The DT, KNN, SVM, and RF classifiers were using BioOne-Hot preprocessed sequences of OVA, Tras-Positive, Tras-Negative, Rani-Positive, and Rani-Negative. The classifiers were trained to distinguish the sequences into five classes.

In the experimental settings 1, 2, and 3, the bio-physicochemical properties of isoelectric, volume, hydrophobicity, solubility, charge, and solvent accessible surface area (SASA) were determined using a grid search approach to obtain the best combination of the bio-physicochemical properties with the highest impact on the classifiers' performance improvement.

To evaluate the impact of the preprocessing method on improving antibody classification, the metrics of (a) Accuracy (ACC), (b) Sensitivity (Sn), (c) Specificity (Sp), and (d) F-measure were calculated as follows:

$$ACC = \frac{( TP + TN )}{TP + FN + FP + TN )} \\ Sn = \frac{TP}{( TP + FN )} \\ Sp = \frac{TN}{FP + TN} \\ F - measure = \frac{( 2 \times PR \times RC )}{( PR + RC )}$$

where TP is a true positive, TN is a true negative, FP is a false positive, and FN is a false negative. Precision (PR) and Recall (RC) are equivalent to sensitivity (Sn) and specificity (Sp) respectively.

The Area Under the Receiver Operating Characteristic (ROC) curve, or AUROC, was used to further evaluate the performance of the classifiers. The AUROC is equal to the probability that the predictor may rank a randomly chosen positive sample higher than a randomly chosen negative sample. The AUROC is in the range of 0.5 and 1, where 0.5 indicates that a classifier does not perform better than random guesses, and where 1 indicates that the classifier perfectly separates positive samples from negative samples (i.e., with 100% classification accuracy).

Amongst the three padding methods, the pre-padding showed an improvement in the classifier's performance. Therefore, the pre-padding method was used to complete experiments 1, 2, and 3. Amino acids' bio-physicochemical properties of charge and hydrophobicity may play a significant role in an antibody's specificity and binding to its target. When the charge, hydrophobicity, and volume were implemented in combination as the defined value in the BioOne-Hot encoding, the highest improvements were obtained in antibody classification performance based on antibodies' specificity and binding to their targets.

Trastuzumab is an antibody targeted against the HER2 tyrosin kinase receptor to treat breast cancer. Tras-Positive and Tras-Negative CDR-H3 sequences were classified into two classes based on their binding strength to ERBB2 receptor to a region in domain IV of the extracellular domain. The DT, KNN, SVM and RF classifiers were trained on the Tras-Positive and Tras-Negative CDR-H3 sequences that were preprocessed with the BioOne-Hot encoding implemented with charge, hydrophobicity, and volume. The classifiers were trained to distinguish the sequences into two classes of strong binder and weak binder to HER2 receptor. The classification metrics of Sn, Sp, F-measure, and ACC for DT-BioOne-Hot, KNN-BioOne-Hot, SVM-BioOne-Hot and RF-BioOne-Hot increased as compared to the baseline experiments of the DT-One-Hot, KNN-One-Hot, SVM-One-Hot, and RF-One-Hot. The K-Nearest Neighbors' accuracy in the KNN-BioOne-Hot of experiment 1 showed a 3.4% improvement compared to its baseline experiment (Table 4). In these experiments, AUROC increased by 1% for the DT, KNN, and RF models, when the CDR-H3 sequences were preprocessed by the BioOne-Hot (FIG. 17A, Panels A1-C2), while the AUROC for the SVM model remained the same compared to its baseline experiment (FIG. 17A, Panels D1-D2).

TABLE 4
Classification Models Comparison. Classification metrics of Sn, Sp, F-measure, and ACC are reported on DT, KNN,
SVM, and RF classification models when BioOne-Hot is used to process the CDR-H3 sequences data compared to One-Hot.

| | 2 Classes | | | | 4 Classes | | | | 5 Classes | | | |
|---|---|---|---|---|---|---|---|---|---|---|---|---|
| Classification | Classification Metrics | | | | Classification Metrics | | | | Classification Metrics | | | |
| Models | Sn | Sp | F-measure | ACC | Sn | Sp | F-measure | ACC | Sn | Sp | F-measure | ACC |
| DT-BioOne-hot | 0.715 | 0.715 | 0.715 | 0.716 | 0.645 | 0.623 | 0.598 | 0.652 | 0.610 | 0. [symbol] | 0. [symbol] 15 | 0. [symbol] 4 |
| DT-one-hot | 0.701 | 0.699 | 0.597 | 0.698 | 0.588 | 0. [symbol] | 0.520 | 0.569 | 0.552 | 0.541 | 0. [symbol] | 0. [symbol] 39 |
| KNN-BioOne-hot | 0.772 | 0.760 | 0.7588 | 0.761 | 0.772 | 0.760 | 0.758 | 0.761 | 0.736 | 0.732 | 0.730 | 0.732 |
| KNN-one-hot | 0.748 | 0.728 | 0.722 | 0.727 | 0.748 | 0.728 | 0.722 | 0.727 | 0.724 | 0.723 | 0.720 | 0.717 |
| SVM-BioOne-hot | 0.799 | 0.799 | 0.797 | 0.797 | 0.724 | 0.722 | 0.722 | 0.753 | 0.763 | 0.764 | 0.762 | 0.766 |
| SVM-one-hot | 0.789 | 0.798 | 0.797 | 0.787 | 0.707 | 0.706 | 0.705 | 0.739 | 0.765 | 0.766 | 0.763 | 0.764 |
| RF-BioOne hot | 0.784 | 0.783 | 0.783 | 0.784 | 0.712 | 0.703 | 0. [symbol] | 0.700 | 0.706 | 0.708 | 0.70 [symbol] | 0.707 |
| RF-one-hot | 0.780 | 0.729 | 0.779 | 0.779 | 0.709 | 0. [symbol] | 0. [symbol] | 0. [symbol] | 0. [symbol] | 0. [symbol] | [symbol] | 0.703 |

[symbol]

indicates data missing or illegible when filed

Ranibizumab is an antibody that binds to and inhibits vascular endothelial growth factor A (VEGF-A). Rani-Positive and Rani-Negative CDR-H3 sequences were classified into two classes based on their binding strength to VEGF-A receptor. Ranibizumab is used to treat age-related macular degeneration, macular edema following retinal vein occlusion, diabetic macular edema, myopic choroidal neovascularization, and diabetic retinopathy. In experiment 2, the DT, KNN, SVM and RF classifiers were trained on the Tras-Positive, Tras-Negative, Rani-Positive, Rani-Negative CDR-H3 sequences. The CDR-H3 sequences were preprocessed with the BioOne-Hot encoding with implemented charge, hydrophobicity, and volume values. The classifiers were trained to distinguish the sequences into four classes of strong binder and weak binder to HER2 receptor and strong binder and weak binder to VEGF-A receptor. The classification metrics of Sn, Sp, F-measure, and ACC for DT-BioOne-Hot, KNN-BioOne-Hot, SVM-BioOne-Hot and RF-BioOne-Hot increased as compared to the baseline experiments. In this experiment, the Decision Tree's accuracy in the DT-BioOne-Hot on classifying 4 classes of Tras-Positive, Tras-Negative, Rani-Positive, and Rani-Negative showed an 8.3% improvement compared to its baseline (Table 4). The DT-BioOne-Hot demonstrated the maximum improvement by the BioOne-Hot preprocessing approach in experiment 2. The AUROC increased by 5% for the DT model (FIG. 17B, Panels E1-E2), 1% for the KNN model (FIG. 17B, Panels F1-F2), while it remained the same for the SVM model (FIG. 17B, Panels G1-G2), and the RF model (FIG. 17B, Panels H1-H2).

Ovalbumin is usually used to sensitize immune reactions and binds to major histocompatibility complex class II I-A(d) protein (MHC class II molecules (I-Ad)). OVA CDR-H3 sequences were included in the input data on experiment 3, and the DT, KNN, SVM, and RF classifiers were trained on the OVA, Tras-Positive, Tras-Negative, Rani-Positive, and Rani-Negative CDR-H3 sequences. The CDR-H3 sequences were preprocessed with the BioOne-Hot encoding with implemented charge, hydrophobicity, and volume values. The classifiers were trained to distinguish five classes of strong binder and weak binder to HER2 receptor and strong binder and weak binder to VEGF-A receptor and strong binder to MHC class II molecules (I-Ad) by the classifiers. In experiment 3, the classification metrics of Sn, Sp, F-measure, and ACC increased when the CDR-H3 sequences were preprocessed with the BioOne-Hot encoding. The SVM-BioOne-Hot and RF-BioOne-Hot demonstrated the maximum improvement in classifying 5 classes of OVA, Tras-Positive, Tras-Negative, Rani-Positive, and Rani-Negative CDR-H3 sequences with improved ACC of 1.5%. Moreover, the AUROC increased by 4% for the DT model (FIG. 17C, Panels I1-I2), 1% for the KNN model (FIG. 17C, Panels J1-J2), and 1% for the RF model (FIG. 17C, Panels L1-L2), while it remained the same for the SVM model (FIG. 17C, Panels K1-K2).

The results demonstrated the successful impact of the BioOne-Hot in classification models' improvement. The KNN model demonstrated higher improvement in differentiating Tras-Positive stronger binding to HER2 when the sequences were processed using the BioOne-Hot preprocessing. The BioOne-Hot preprocessing further shows maximum improvements in the DT model for classifying Trastuzumab binding strength to HER2 and Ranibizumab binding strength to VGEF-A as well as differentiating the Trastuzumab and Ranibizumab. Using systems and methods of the present disclosure, antibody/nanobody lead candidates may be validated through their specificity and binding to a given antigenic target via more accurate classifier screening.

### REFERENCES

[1] Derek M Mason, Simon Friedensohn, C'edric R Weber, Christian Jordi, Bastian Wagner, Simon M Meng, Roy A Ehling, Lucia Bonati, Jan Dahinden, Pablo Gainza, et al. Optimization of therapeu-tic antibodies by predicting antigen specificity from antibody sequence via deep learning. Nature Biomedical Engineering, 5(6):600-612, 2021 is incorporated by reference herein in its entirety.
[2] Ge Liu, Haoyang Zeng, Jonas Mueller, Brandon Carter, Ziheng Wang, Jonas Schilz, Geraldine Horny, Michael E Birnbaum, Stefan Ewert, and David K Gifford. Antibody complementarity determining region design using high capacity machine learning. Bioinformatics, 36(7):2126-2133, 2020 is incorporated by reference herein in its entirety.
[3] Leonard D Goldstein, Ying-Jiun J Chen, Jia Wu, Subhra Chaudhuri, Yi-Chun Hsiao, Kellen Schneider, Kam Hon Hoi, Zhonghua Lin, Steve Guerrero, Bijay S Jaiswal, et al. Massively parallel single-cell b-cell receptor sequencing enables rapid discovery of diverse antigen-reactive antibodies. Communications biology, 2(1):1-10, 2019 is incorporated by reference herein in its entirety.
[4] Khalilian Saeed., Moti Zahra., Baloochestani Arian., Hallaj Yeganeh., Chavosh AliReza., and Hemmatian Zahra. Vaerestl: A novel generative model for designing complementarity determin-ing region of antibody for sars-cov-2. In Proceedings of the 15th International Joint Conference on Biomedical Engineering Systems and Technologies—BIOINFORMATICS, pages 107-114. IN-STICC, SciTePress, 2022 is incorporated by reference herein in its entirety.
[5] J. R. Quinlan Introduction to Decision Trees-Machine Learning 1: 81-106, 1986 is incorporated by reference herein in its entirety.
[6] Aeberhard, S., Coomans, D., de Vel, O. Comparison of Classifiers in High Dimensional Settings. Tech. Rep. no. 92-02, (1992), Dept. of Computer Science and Dept. of Mathematics and Statistics, James Cook University of North Queensland, 1992 is incorporated by reference herein in its entirety.
[7] Support-Vector Networks, Corrina Cortes, Vladimir Vapnik, Machine Learning, 20, 273-297 (1995) is incorporated by reference herein in its entirety.
[8] Breiman, L. Random Forests. Machine Learning 45, 5-32 (2001). https://doi.org/10.1023/A:1010933404324 is incorporated by reference herein in its entirety.
[9] Baldi et al., 2000. Baldi, P., Brunak, S., Chauvin, Y., Andersen, C. A., and Nielsen, H. (2000). Assessing the accuracy of prediction algorithms for classification: an overview. Bioinformatics, 16(5):412-424 is incorporated by reference herein in its entirety.
[10] Matthews, 1975. Matthews, B. W. (1975). Comparison of the predicted and observed secondary structure of t4 phage lysozyme. Biochimica et Biophysica Acta (BBA)-Protein Structure, 405(2):442-451 is incorporated by reference herein in its entirety.
[11] Chen P J, Lin M C, Lai M J, Lin J C, Lu H H, Tseng V S. Accurate Classification of Diminutive Colorectal Polyps Using Computer-Aided Analysis. Gastroenterology. 2018 February; 154(3):568-575. doi: 10.1053/j.gastro.2017.10.010. Epub 2017 Oct 16. PMID: 29042219 is incorporated by reference herein in its entirety.
[12] Feng, Z., Wen, H., Bi, R. et al. A clinically applicable molecular classification for high-grade serous ovarian cancer based on hormone receptor expression. Sci Rep 6, 25408 (2016). https://doi.org/10.1038/srep25408 is incorporated by reference herein in its entirety.
[13] Zhang, Chen, Li, Qiuchi, Song, Dawei, Aspect-based Sentiment Classification with Aspect-specific Graph Convolutional Networks, Proceedings of the 2019 Conference on Empirical Methods in Natural Language Processing and the 9th International Joint Conference on Natural Language Processing (EMNLP-IJCNLP), 2019, doi=10.18653/vl/D19-1464 is incorporated by reference herein in its entirety.
[14] Hajian-Tilaki K. Receiver Operating Characteristic (ROC) Curve Analysis for Medical Diagnostic Test Evaluation. Caspian J Intern Med. 2013 Spring; 4(2):627-35. PMID: 24009950; PMCID: PMC3755824 is incorporated by reference herein in its entirety.

### Example 5: Computational Antibody Design Methodology for ML-Based Design of Antibody Variable Region Residues (Fvs), Including CDRs and FRs, and Discovery of Antibody Therapeutic Hits with Drug-Like Properties

Methods and systems of the present disclosure are used to perform improved classification of antibody sequences, using data preprocessing approaches such as one-hot encoding. The methods and systems may comprise a fast, high-throughput, and precise computational antibody design methodology for accurate machine learning based design of antibody variable region residues (Fvs), including CDRs and FRs, to discover antibody therapeutic hits with drug-like properties. The methods and systems may comprise AI-based algorithms to predict antibody paratope 3D structure, analyze antibodies for their biological and key therapeutic properties, and/or identify antibody therapeutics with optimal drug-like properties.

Antibody (Ab) therapeutics are becoming an essential class of biologics used to treat known and novel diseases due to their unique specificity and efficacy. However, the long time and the high cost associated with developing therapeutic antibodies present key challenges in the availability of antibody therapeutics despite the increasing need for these therapies. Moreover, given the massive antibody space and existing methods' limitations, numerous antibody mutations may be yet to be discovered for known or novel diseases³.

In contrast to other approaches, in-silico antibody library design methods may offer fast, efficient, accurate, and low-cost discovery approaches to boost the speed of antibody discovery, while exploring a massive antibody space that may not be feasible through other approaches. In antibody therapeutics, the complementarity determining regions (CDRs) of the variable region residue (Fv) play a significant role in antibody specificity and antibody binding to a target. However, besides CDRs, other fractions of the Fv region, including frameworks (FWRs), play a substantial role in determining the therapeutic properties of antibodies. For example, mutations in the FRs may lead to a shifted CDR, which can impact antibody binding ability. Furthermore, an FR's mutations may lead to better antibody binding by decreasing the entropy and antibody biochemical properties such as stability, charge, and hydrophobicity. Other in-silico library generation approaches may focus merely on designing antibodies' CDRs, specifically the CDR3 of heavy chains (CDR-H3), and cannot design the entire variable regions. Furthermore, those approaches may not humanize the Fv region of the designed antibodies to alleviate unwanted therapeutic properties, which may trigger immune responses that can lead to antidrug antibody formation. Thus, the rapid design and humanization of the antibody's Fv, composed of seven amino acid regions, in which four are the frameworks (FWRs), and three are the complementarity determining regions (CDRs), may be of critical importance. A deep learning-based model was developed to design new antibody sequences against a novel antigenic target. The deep learning model offers significant advantages over other models, such as its unique ability to rapidly and accurately design CDR-H3 sequences when the input data is scarce for a novel or existing target. The model was used for designing new antibodies against SARS-CoV-2 and successful development of an antibody CDR-H3 hit generation tool. Further, the extracted key features offered by the deep learning model were used to analyze antibody sequences and for successful demonstration of an antibody's specificity and cross-reactivity to one or multiple targets.

Using systems and methods of the present disclosure, the following may be achieved:

Developing a novel computational model to design and humanize Fvs of the heavy chain (VH) and light chain (VL) for novel and/or difficult targets prioritized with specific proteins (e.g., SARS-CoV-2).

Demonstrating the scalability of the computational model in designing humanized antibodies against diverse targets.

Assessing biological values of the humanized antibody FVs predicted by the computational model.

Systems and methods of the present disclosure may be used to accurately design humanized antibody variable region residues (Fvs), including CDRs and FRs, to discover the best humanized antibody therapeutic hits with drug-like properties through a fast, high-throughput, and precise computational antibody design methodology. The approach may use a generative model that can learn the transformative relationships of antibody sequences directly from the complex and high-dimensional amino acid sequences space. The generative model can design new and highly diverse humanized sequence variants with improved physicochemical properties. A few baseline models are adapted to evaluate the performance of the model compared to the baseline models. Evaluation metrics, such as accuracy, sensitivity, and specificity, are reported on the predicted sequences to assess the biologically valuable predicted humanized antibodies' VH and VL. To increase the scalability of the AI engine, the deep generative model is trained using antibody sequences against various antigens.

These capabilities can be demonstrated through the following processes:
1. Acquire antibody sequence datasets.
2. Develop and train a deep generative model to design and humanize antibody FVs.
3. Develop an automated AI engine that designs humanized antibody Fv sequences, including FRs, and CDRs for both heavy chain (VH) and light chain (VL).
4. Evaluate the deep generative model performance.
5. Assess the quality of designed humanized antibody sequences of the VH and VL.

Generally, the limited availability of massive training data may constrain progress in deep generative and computational design. However, systems and methods of the present disclosure were demonstrated to be capable of designing biologically valuable antibody CDR-H3 sequences against SARS-CoV-2 despite training data shortage.

By using systems and methods of the present disclosure, a cost and time-effective approach may be developed, while reducing the risks associated with other antibody therapeutics discovery process. In addition, an AI technology approach may be developed to improve the design quality of humanized antibodies. As demonstrated by new antibody sequences discovery results against SARS-CoV-2, systems and methods of the present disclosure may be used to accurately design and humanize antibody variable regions for novel and difficult targets (such as SARS-CoV-2), which can enhance the rapid response ability against a future pandemic.

Moreover, the AI-based tool is also capable of discovering therapeutic antibodies against various disease targets, such as cancer targets. Therefore, systems and methods of the present disclosure may be used to develop effective and safe therapeutic antibodies for single antibody therapy, combination antibody therapy, and the design of next-generation antibody therapeutics may be considerably enhanced.

### Example 6: AI-Based Algorithms to Predict Antibody Paratope 3D Structure, Analyze Antibodies for their Biological and Key Therapeutic Properties, and/or Identify Antibody Therapeutics with Optimal Drug-Like Properties

Antibody (Ab) therapeutics are becoming an essential class of biologics used to treat known and novel diseases due to their unique specificity and efficacy. However, the long time and the high cost associated with developing therapeutic antibodies may present key challenges.

Using methods and systems of the present disclosure, antibodies' paratope 3D structure are computationally predicted against given targets for a more precise antibody therapeutics design. Antibodies' multifaceted properties through their interactions with specific epitopes make them an important modality in cancer treatment. For example, antibodies against Human epidermal growth factor receptor 2 (HER2) may be utilized for breast cancer treatment. To increase the effectiveness of systemic treatment through targeted therapy, accurate antibody design is required to identify an antibody that specifically binds to a given antigen. Moreover, the antibody's specificity and binding affinity are ruled by the 3D binding interface between the antibody residues (paratope) and the antigen residues (epitope). Thus, modeling the 3D structure interaction between an epitope and a paratope is critical for designing the best lead antibody candidates in-silico. However, other computational models for designing antibody paratopes do not model the 3D structure interactions between the antibody paratope and the given antigen epitope. Furthermore, antibody leads identification through antibody hits analysis requires biological properties analysis, which is difficult to put into practice by experimental techniques as they need an extensive set of tests. In addition, an optimal selection of antibody therapeutics with drug-like properties requires key therapeutic properties to be considered, similarly to fitting puzzle pieces together. These extensive wet-lab experimental testing and antibody drug-like selection are costly and time-consuming. In contrast, in-silico analysis methods offer faster and more efficient approaches to rigorously analyze and validate antibody candidates from the early stage of the discovery process to mitigate the risk of drug failure later on during clinical testing.

Using methods and systems of the present disclosure, novel deep learning models are developed that disentangle an antibody paratope sequence together with its associated side-chain and backbone 3D structure for any given antigen to design and analyze antibody leads more precisely. An example is applied to breast cancer treatment through anti-HER2 antibodies. Specific applications are as follows:

Specific Application 1. Develop deep learning-based algorithms to design antibody paratopes and predict their 3D structures. These studies are designed to develop deep learning algorithms to generate antibody paratopes and predict their 3D structures. The approach is a conditional model where a known target antigen is conditioned for our generative model. We assume the antigen 3D structure and its epitope are given as input for our AI engine. Then we design the paratope residue and its 3D structure that binds to the given epitope. We adopt different baseline models to evaluate the performance of our model compared to the baseline models. To measure contact prediction accuracy, we also report evaluation metrics on the predicted paratopes, such as accuracy, ROC, and AUC—milestone: to reduce the antibody drug discovery time by >10×.

Specific Application 2. Develop Deep Learning Models to analyze antibodies for their biological and key therapeutic properties. To assess the designed antibodies for lead identification and lead optimization, we develop deep learning-based models to analyze antibodies' binding affinity, epitope-paratope interaction, clearance, viscosity, solubility, stability, immunogenicity, developability, affinity maturation, manufacturability, half-life, pharmacokinetic, yield, aggregation, function, humanization, and humaneness. We train our deep learning-based models using predicted antibody paratope 3D structures and antibody paratope sequences. We use quality metrics to estimate the predicted values such as accuracy, sensitivity, and specificity—milestone: to complete the above-mentioned biological and key therapeutic properties analysis with at least 90% accuracy and sensitivity.

Specific Application 3. Identify antibody therapeutics with optimal drug-like properties through an automated and multimodal screening method. To identify the shortlist of antibody therapeutics with optimal drug-like properties, we develop an ensemble model by utilizing the biological and key therapeutic properties offered by our deep learning models. We also formulate developability and simulation methods to evaluate the selected antibodies with optimal drug-like properties in-silico-milestone: to automate antibody analysis for rapid and multimodal antibody therapeutics identification.

Results of the above applications include: (a) establish deep learning-based antibody design tool that can significantly improve the pre-clinical antibody discovery efficiency by reducing the time, the cost, and the risks associated with antibody drug discovery and development, thereby shortening the time patients will have to wait for the drugs, (b) establish a solid AI-based tool to rapidly analyze antibody candidates against novel or existing antigenic targets, (c) offer fully automated AI-based tool capable of discovering therapeutic antibodies for breast cancer treatment.

Using the applications above, developing effective and safe therapeutic antibodies for single antibody therapy, combination antibody therapy, and the design of next-generation antibody therapeutics are considerably enhanced.

To identify therapeutic antibody candidates against a target, the following discovery and development operations may be required prior to the clinical stage: antibody discovery and screening based on the antibody's binding ability and specificity to antigenic targets, antibody lead identification based on the antibody's biological properties, and antibody lead optimization to enhance the balance between safety, efficacy, and manufacturability [1]. However, these may require costly, laborious, and time-consuming conventional wet-lab experimentations to design, screen, and analyze antibodies for their biological and key therapeutic properties, which only in the pre-clinical stage may take about 6 years and may cost over $800M [2]. Nonetheless, AI-based models can design and analyze antibodies for their biological and therapeutic properties more accurately and more efficiently at a significantly lower cost and a considerably shorter time [3,4,5,6]. In-Silico antibody therapeutics design requires designing an antibody that specifically binds to a given antigen [7,8]. Moreover, the antibody's specificity and binding affinity are ruled by the 3D binding interface between the antibody residues (paratope) and the antigen residues (epitope). Thus, modeling the 3D structure interaction between an epitope and a paratope is critical for designing the best lead antibody candidates in-silico [9]. However, the majority of existing computational models for designing antibody paratopes do not consider modeling the 3D structure interactions between the antibody paratope and the given antigen epitope [10,11]. Furthermore, antibody leads identification through antibody hits analysis requires biological properties analysis, which is extremely difficult to put into practice through experimental techniques as they need an extensive set of tests. In addition, an optimal selection of antibody therapeutics with drug-like properties requires key therapeutic properties to be screened [12,13]. The extensive wet-lab experimental testing and antibody drug-like selections are costly and time-consuming. Nonetheless, In-silico analysis of designed antibodies and antibody drug-like selections in the early discovery phase [14] is a rigorous optimization and validation stage and a preemptive operation that is a lot less expensive and faster than discovering the potential drug failure later on during the clinical testing.

To date, we have successfully developed a deep learning-based model to design new antibody sequences against a novel antigenic target [8]. The deep learning model offers significant advantages over other models, such as its unique ability to rapidly and accurately design the third complementarity determining regions in the heavy chain (CDR-H3) sequences when the input data is scarce for a novel or existing target. We completed pressure testing our AI technology on designing new antibodies against antigenic targets and successfully achieved an antibody CDR-H3 hit-generation tool. We further utilized the extracted key features offered by the deep learning model to analyze antibody sequences and successfully analyzed antibody's specificity and cross-reactivity to one or multiple targets for lead identification purposes [14].

Using methods and systems of the present disclosure, approaches based on deep learning technologies can provide a solution to the growing need for rapid, high-resolution, and efficient antibody design and its challenges. Our deep learning-based model autoregressively generates antibody paratope sequences by receiving an antigen structure and its epitopes while recursively improving antibody paratope's backbone and side chain structure. We also develop a deep learning-based analysis model to rapidly and accurately analyze antibodies based on their biological properties including binding affinity, epitope-paratope interaction, clearance, viscosity, solubility, stability, immunogenicity, developability, affinity maturation, manufacturability, half-life, pharmacokinetic, yield, aggregation, function, and humanization and humaneness. In addition, we develop an ensemble method for a multimodal screening to identify antibody therapeutics with optimal drug-like properties through an automated method. We use the interaction between the human epidermal growth factor receptor (HER2) and anti-HER2 antibodies as a well-studied antibody-antigen interaction to develop our proposed deep learning models27-31. There is an extensive availability of anti-HER2 antibody data which in combination with our proprietary in-silico-designed anti-HER2 antibody data enable us to rigorously train our deep learning models.

In-silico antibody design methods can rapidly and accurately generate antibodies that bind to a given antigen. Binding affinity depends on how a paratope matches an epitope like the typical example of lock and key. A high-quality epitope-paratope match results in a high affinity at the binding interaction of antibody:antigen. In contrast, there is no affinity to the given antigen when there is no match between an epitope and a paratope (FIG. 18). Thus, finding the best epitope-paratope match is an essential task in antibody design and discovery which can be achieved through studying the interaction of antibody paratopes 3D structure with the antigen epitopes 3D structure. Antibodies retain a diverse set of clinically significant properties because they can bind to different epitopes of an antigen, however, some of these antibodies may establish a more desirable binding through a unique mechanism of action which can result in unique therapeutic properties. Thus, conditioning the antibody design process to a specific antigen epitope provides a controllable and adaptable antibody design capability. Moreover, screening the designed antibodies based on their key biophysical and therapeutics properties which should be rigorously studied at the earliest stage of the antibody therapeutics discovery process is lengthy, inefficient, and costly. Therefore, the need for automated, accurate, fast, and scalable technologies in antibody analysis and epitope prediction is rising. Here, we develop in-silico technologies that can accurately and efficiently design antibody therapeutics by generating new antibody paratopes and predicting their 3D structures. In addition, we propose in-silico methods and systems to analyze the designed antibodies through automated and multimodal screening approaches.

Specific innovations of the approach include the following.

Integration of deep learning models for high-quality design of binders through accurate antibody paratope generation and prediction of their 3D structure. Current in-silico paratope folding and 3D structure prediction models cannot provide flexible modeling capabilities to design antibody mutations while predicting the paratope structures [15,16]. Moreover, previous models are not capable of modeling both antigen and antibody simultaneously and do not consider the rotation of epitope in the space [17]. In addition, current in-silico atom-based level 3D structure and design models are either computationally very expensive or just model the backbone and ignore the side-chain conformation [18]. However, a deep learning technology that can generate and design antibody paratope sequences and 3D structures conditioned to a given antigen while modeling both backbones and side-chains, promises a flexible, efficient, and high throughput approach for rapid, and accurate contact prediction and paratope design.

Modeling the antigen epitope and antibody paratope simultaneously to test the antibody paratope-antigen epitope interactions. In contrast to other computational models, the technology models a given antigen epitope using its sequences and 3D structure to generate antibody sequences and to predict antibodies paratope 3D structures that have binding interactions with the given antigen faster and more accurately.

Integration of deep learning models for analyzing the predicted paratopes and their 3D structure for their biological and therapeutic properties. We establish a deep learning-based model to further assess the predicted contacts and designed paratopes for their key biological and therapeutic properties.

Development of an automated and ensemble model for multimodal screening. The in-silico approach is armed with an automated ensemble method to filter out the undesirable antibodies and identify the desirable antibodies with optimal drug-like properties. The ensemble model selects among antibody paratopes interacting with the antigen epitopes the best lead candidates that have the full spectrum of desirable key therapeutics and biological properties. This results in a highly efficient paratope 3D prediction and identification of the antibody lead candidates.

Systematic analysis for antibody paratope-antigen epitope interaction modeling. We recruit evaluation metrics, including accuracy, specificity, and sensitivity, to validate our deep learning and ensemble models. While screening antibody competition with respect to a specific antigen epitope, we combine competition index, biological, and machine learning metrics to better characterize the designed antibody paratopes and their predicted 3D structures.

Modeling Anti-HER2 antibodies and HER2 receptor interactions using the deep learning-based model. Overexpression of human epidermal growth factor receptor (HER2) may occur in breast cancer patients and anti-HER2 antibodies may have robust anti-tumor activity in patients with HER2-overexpressing breast cancer. Moreover, in addition to the extensive availability of anti-HER2 antibody data against HER2 variant epitope domains, HER2 receptor comprises variant epitope domains that have binding interactions with various antibodies leading to different mechanisms of action (MOAs) [22-26]. As a result, the interaction between anti-HER2 antibodies and HER2 receptor can serve as an excellent model for developing our deep learning-based antibody paratope generation and 3D structure prediction model for designing and discovering the best-in-class antibody therapeutics for breast cancer treatment.

We pursue complementary approaches for systematic and efficient design of antibody paratopes and their associated 3D structures for a given antigen to design best-in class antibody therapeutics. To do so, we develop different deep learning and artificial intelligence models and train them by using both natural and designed antibody sequences to achieve a model with high scalability (FIG. 19). Our Deep learning technology predicts antibody binding affinity by modeling antigen epitope-antibody paratope interactions. Our model also generates new antibody paratopes by generating one residue at a time and predicting their associated 3D structures with both backbones and side chains. We also develop deep learning-based algorithm to evaluate the generated paratopes and predicted contacts. We also develop deep learning-based models for automated analysis and screening of the generated antibody paratopes and their predicted structures for their biological and therapeutic properties to identify the best-in-class antibodies with drug-like properties.

Application 1. Develop deep learning-based algorithms to design antibody paratopes and predict their 3D structures

In-silico model for designing disruptive antibody paratope mutations [8]. We have successfully developed a deep learning-based model, VAEResTL, to design new antibody sequences and predict antibody mutations specific to a novel antigenic target (FIG. 20). We got to leverage the power of deep learning algorithms to predict therapeutic antibodies/nanobodies against a novel and difficult target. We presented a novel end-to-end generative “VAEResTL” model to discover amino acid sequences against specific target antigens for which the available training data was very limited. The VAEResTL model is based on a Convolutional Variational Autoencoder (VAE) model, a Residual Neural Network (Resnet) structure, and a Network-based Transfer Learning (TL) technique. We demonstrated Resnet improved our deep generative model VAERes's performance efficiently in designing and predicting antibody/nanobody amino acid sequences of the third complementarity determining regions in the heavy chain (CDR-H3) by preventing vanishing gradient problems while providing a deep neural network capable of learning CDR-H3 sequence complexity. We further exhibit in our VAEResTL, the learning from our VAERes pre-trained on sufficient antibody amino acid CDR-H3 sequences for different target antigens that can be efficiently transferred for predicting antibody/nanobody amino acid sequences against a new target with appropriate properties. The in-silico library of predicted antibody paratope residues of CDR-H3 sequences by VAEResTL were then subject to in-silico screening and filtering for developability parameters such as viscosity, clearance, solubility, stability, and immunogenicity, resulting in antibody paratope residues of CDR-H3 sequences as lead candidates with optimal therapeutic properties.

The study demonstrates a unique ability to rapidly and accurately design the antibody paratope residues of CDR-H3 sequences against a novel target when the input training data is scarce. Our systematic deep learning algorithm development and validation demonstrate Resnet and transfer learning extend our in-silico generative model capabilities by extending a large library of new and valid antibody paratopes. Our statistical analysis demonstrate that our solo convolutional VAE algorithm could not learn the complexity of antibody paratope residue of CDR-H3 sequences, and 100% of the generated amino acid sequences were abnormal (invalid padding or invalid characters) and inappropriate for further analysis. Nevertheless, when we incorporated Resnet structure into our convolutional VAE, our VAERes model demonstrated significant improvements in generating antibody paratope residue of CDR-H3 sequences for a given target. When we trained our VAERes on training antibody paratope residue of CDR-H3 sequences, 98% of the generated sequences were valid (valid padding with valid characters), and only 2% of the generated sequences were abnormal (invalid padding or invalid characters). Notwithstanding, 86.8% of the valid generated antibody paratope residue of CDR-H3 sequences were duplicate sequences, and only 11.2% of the generated sequences were unique. These results suggest that VAERes could learn a small range of antibody paratope residue of CDR H3 sequences, reflecting the lack of adequate training data for a new target for which the training data was scarce. With VAEResTL, 100% of the generated antibody paratope residue of CDR-H3 sequences were valid (valid padding with valid characters), 70.7% of the valid generated sequences were unique, and only 29.3% of the CDR-H3 sequences were duplicate sequences.

We further compared three baseline generative models' with our deep learning engine model and the designed antibody paratope residue of CDR-H3 sequences (Table 5). The overall machine learning (ML) and biophysical metrics demonstrate our deep generative model, VAEResTL, can more efficiently predict antibody paratope residue of CDR-H3 sequences with binding ability to the antigenic target (Table 5) compared to the other baseline models. We measured the biophysical properties of designed CDR-H3 sequences compared to the biological CDR-H3 sequences and validated the accuracy of our model in designing novel antibody sequences with valuable biophysical properties (FIG. 21, panels A-D). This observation may suggest that VAERes with TL perform well in capturing the charge patterning, H, and composition within generated sequences. We further analyzed the diversity of the generated sequences in terms of their shared n-grams (Sn). The n-gram similarity is lower for VAEResTL with respect to VAERes (Sₙ^{VAEResTL}/Sₙ^{VAERes}>1) for n>2. 2-gram, 3-gram, 4-gram, 5-gram are 0.91, 0.78, 0.63 and 0.44 respectively. These results imply that VAEResTL-generated sequences show strong long-range diversity; however, they are still consistent with biological sequences, as evident from the sequence similarity comparison (Table 5). The VAEResTL-generated antibody paratopes residue of CDR-H3 sequences show more substantial diversity at higher n-grams (lower Sn values) as a desirable feature that can prevent drug resistance while designing next-generation antibody therapeutics for oncology and can provide a larger and more diverse pool of antibody paratopes residue of CDR-H3 sequences for in-silico screening and therapeutics development studies. We also found from the ML visualization heatmaps (FIG. 22, panels A1-C1) and logo plots (FIG. 22, panels A2-C2) that although VAEResTL changed certain CDR-H3 amino acid positions and their frequency distributions, the overall pattern of VAEResTL-generated sequences are closer to seed sequences as compared to the VAERes-generated CDR-H3 sequences (FIG. 22). We tested our deep generative model across various antigenic targets and proved the scalability of our model on antibody sequence design against a specific target. Our deep-learning design model provides a large library of CDR-H3 sequences mutations that can have binding specificity to different epitopes of a given antigenic target. Importantly, the antibody mutations designed by our model can extend our model for antibody paratope design and 3D structure prediction to study the antibodies' binding competition with respect to appropriate epitope residues on the antigen surface.

TABLE 5
Baseline models comparison. Biological Characteristics and Machine
Learning Metrics for generated CDR-H3 sequences by VAERes,
VAEResTL, HMM, LSTM, and Seed sequences

| | | | | | | Machine Learning Metrics | | | |
|---|---|---|---|---|---|---|---|---|---|
| | Biophysical Characteristics | | | | | BLEU | | | |
| Method | NW | H | ISO | Charge | Stability | (2-gram) | (3-gram) | (4-gram) | JSD |
| VAERes | [symbol] | [symbol] | [symbol] | [symbol] | [symbol] | [symbol] | [symbol] | [symbol] | [symbol] |
| VAEResTL | [symbol] | [symbol] | [symbol] | [symbol] | [symbol] | [symbol] | [symbol] | [symbol] | [symbol] |
| LSTM | [symbol] | [symbol] | [symbol] | [symbol] | [symbol] | [symbol] | [symbol] | [symbol] | [symbol] |
| HMM | [symbol] | [symbol] | [symbol] | [symbol] | [symbol] | [symbol] | [symbol] | [symbol] | [symbol] |
| Seed | [symbol] | [symbol] | [symbol] | [symbol] | [symbol] | [symbol] | [symbol] | [symbol] | [symbol] |

[symbol]

indicates data missing or illegible when filed

Our core approach uses antibody paratope residue mutations that have binding specificity to specific antigen epitope targets to design new antibody paratopes and predict their 3D structures. Here, we accurately design antibodies for given antigen residues that contribute to a binding interaction between antibody paratope and antigen epitope leading to a specific mechanism of action. To achieve this goal, we develop and train deep learning models to first predict the contact between the paratope and the epitope. Our model encodes the binding interface of epitope-paratope through a graph neural network approach. The inputs for training our deep learning models are an antigen 3D structure with its specified epitope, and antibody paratope sequences having binding interactions with the given antigen. We assume the 3D structures of the antibody paratopes are not given. For simplicity, we utilize the antibody paratope residue of CDR-H3 sequences on the heavy chain, which is the most variable CDR amongst antibody CDRs and typically contributes the most to antigen specificity in antibodies and nanobodies. Our model predicts the correct residue contacts between the epitopes and the paratopes. Our deep learning model then is conditioned to antigen structure to design new antibody paratope amino acid sequences that bind to the given target and predicts their 3D structure including both backbones and side chains. We focus on HER2 receptor on the surface of the HER2-expressing breast cancer cells and anti-HER2 antibodies that have defined binding labels to specific epitopes of the HER2 while developing our deep learning model. As an example, we focus on antibodies that bind to various domains of HER2 receptor. We test all mutations across a large library of antibody amino acid sequences including the amino acid sequences that we have generated using our proprietary deep learning model [8] and the publicly available anti-HER2 antibody amino acid sequences to generate antibody sequences that interact with specific epitopes on the HER2 surface. Our deep learning-based model is conditioned to HER2 receptor structure and its sequences to generate new anti-HER2 antibody paratopes and predict their structures. With this approach, we can efficiently extract binding information related to the binding specificity of anti-HER2 antibodies to given residues of HER2 receptor to identify the best paratope-epitope matches. The results of our deep learning model can enhance the discovery and design of new antibody therapeutics that block a specific signaling pathway, combat antibody drug resistance, or eradicate the cancerous cell for an effective and safe breast cancer treatment. We adopt different baseline models to evaluate the performance of our proposed model. We report quantitative and qualitative metrics which include but are not limited to accuracy, precision, perplexity (PPL), Area under the ROC Curve (AUC), amino acid recovery (AAR), and contact amino acid recovery (CAAR) to compare the performance of our model with the baseline models for the best epitope-paratope contact prediction and generation outcomes.

We successfully achieved development of a deep learning model for designing novel antibody paratope residue of CDR-H3 sequences against a new target with scarce input data and validated the large library of antibody mutations generated by our proprietary deep learning model through in-silico screening. Using these results, a deep learning model is developed for antibody paratope docking and generation, which demonstrates the accurate and efficient antibody paratope-antigen epitope interaction prediction for the discovery of effective antibody therapeutics for breast cancer treatment. If we do not obtain sufficient contact prediction and paratope generation, we may extend our training data from antibody CDR-H3 sequences to other fractions of paratope residue. Finally, if we obtain little consistency with the contact prediction and paratope design, then we selectively incorporate amino acid's biological properties such as hydrophobicity, volume, or charge into our deep learning model for a highly efficient contact prediction and paratope generation.

Application 2. Develop Deep Learning Models to analyze antibodies for their biological and key therapeutic properties.

A Deep learning model for analysis of antibody sequences [¹⁴]. We have successfully developed a method by utilizing the extracted key features offered by our deep learning model, that is a dimensionality reduction (DR) approach, which we named VAEResDR, (FIG. 23) to analyze antibodies for their two key therapeutic and biophysical properties, specificity and cross-reactivity, to one or multiple targets to identify the antibody lead candidates.

We demonstrated that our scalable and generalizable VAEResDR reduces sequences' high-dimensional space to a two-dimensional visualization space while keeping the important features. The key extracted features are utilized to accurately analyze antibody CDR-H3 sequences and effectively improve clustering. These analyses may suggest whether antibody paratope residue of CDR-H3 sequences have specificity or cross-reactivity to one or multiple targets. To systematically evaluate our model we designed and executed two sets of experiments by using antibodies including Trastuzumab (anti-HER2), Ranibizumab (anti-VEGF), yeast display scFV, and Chicken Ovalbumin (OVA). We trained our deep learning model on three antigenic targets in experiment one. We trained our deep learning model on four antigenic targets in experiment two. We used visualization plots (FIG. 24, panels A-N) and clustering quality metrics (Table 6) to evaluate the impact of our VAEResDR model on antibody amino acid CDR-H3 sequences' DR and clustering as compared to baseline methods.

TABLE 6
Clustering Results: Quality Metrics comparison for K-Means and GMM on
CDR-H3 sequences on PCA, t-SNE, UMPA, VAE, scCCESS (AE), VAEDR
and VAEResDR for three targets, 5 clusters and four targets, 6 clusters

| | Clustering | 5 Cluster | | | | | 6 Cluster | | | | |
|---|---|---|---|---|---|---|---|---|---|---|---|
| Method | Algorithms | ARI | MI | HS | CS | VMS | ARI | MI | HS | CS | VMS |
| PCA | K-Means | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| | GMM | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| t-SNE | K-Means | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| | GMM | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| UMAP | K-Means | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| | GMM | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| VAE | K-Means | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| | GMM | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| scCCESS | K-Means | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| (AE) | GMM | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| VAEDR | K-Means | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| | GMM | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| VAEResDR | K-Means | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| | GMM | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |

[symbol]

indicates data missing or illegible when filed

The 2D visualization plots demonstrate our deep learning analysis tool is capable of well-separating antibody sequence groups with valuable biophysical parameters recognized within the two-dimensional sequences space (FIG. 24, panels G and N). We tested both clustering models of K-Means and GMIM on the extracted features by our VAEResDR compared to baseline models. We demonstrated that our deep learning analysis tool of VAEResDR outperformed PCA, t-SNE, UMAP, and deep learning methods of VAE, scCCESS (AL) and our VAEDR model (Table 6) in both five cluster and six cluster experiments. To better illustrate the performance of DR methods in improving clustering analysis, we further reported the ranks statistics of the compared methods bar chart according to the quality metrics values (FIG. 25, panel A, 5 clusters, and FIG. 25, panel B, 6 clusters). VAEResDR ranked 1, 2, and 3 in 5 cluster experiments (FIG. 25, panel A) and ranked 1 and 2 in 6 cluster experiments (FIG. 25, panel B), demonstrating its best performance. Our VAEDR has a second place for its performance as it contains a distribution of ranks 1, 2, and 3 in 6 cluster experiments (FIG. 25, panel B). The scCCESS (AL) and VAL showed the lowest performance, with a distribution performance rank of 6 and 7 in the 6 cluster experiments (FIG. 25, panel B). The overall bar chart performance rank plots confirmed clustering performance degraded when OVA sequences were included in the input datasets. However, our VALResDR model alongside GMIM effectively showed high performance with improved antibody CDR-H3 sequences clustering.

We then established an analysis method to assess the validity of the antibody specificity and cross-reactivity to one or multiple antigenic targets using biological and machine learning metrics (FIG. 26, panels A-H). The results of our antibody analysis model may suggest that antibody sequences against antigenic target (a) may have specificity to antigenic target (b). Our antibody sequences analysis model may also demonstrate that sequences against antigen target (a) may have cross-reactivity with the target (b) (FIG. 26). The results of these analyses may suggest anti-MHC II antibodies may have binding ability similar to OVA antibodies, thus OVA antibodies may have binding affinity to MHC II receptor. With these analyses, we could prevent any false-positive or false-negative results when performing sequence screening for either of the targets. Our quantitative analysis demonstrates that our model achieved superior DR performance in the two designed experimental series and has significant generalizability on different datasets with diverse structures in the original and high-dimensional spaces. Our model provides a readily usable tool for screening CDR-H3 sequences' specificity and identifying highly specific CDR-H3 sequences that bind to one or multiple antigenic targets.

We establish an analysis model to accurately and efficiently analyze the predicted contacts and designed antibody paratopes provided by our deep learning model. We establish a deep learning-based screening model to analyze every generated antibody paratope mutations and their associated 3D structures against HER2 receptor and measure their binding interactions by further filtering the analysis. We add more functions to our deep learning model to further analyze every generated antibody paratope mutation against HER2 receptor for their clearance, viscosity, solubility, stability, immunogenicity, developability, affinity, affinity maturation, manufacturability, half-life, pharmacokinetic, yield, aggregation, function, humanization, and humaneness. We incorporate a binding interaction index such as minimum residue score into our method to optimize our analysis model. We use visualization and statistical measures to assess our analysis model's performance. We further validate the predicted contacts and the generated paratopes by measuring contact accuracy, interface RMSD, and ligand RMSD. Amino acids physicochemical attributes, including hydrophobicity, polarity, exposed surface, or accessibility, will also be used to assess the predicted paratopes.

Results were shown in developing antibody analysis methods and validation of two therapeutic properties of specificity and cross-reactivity through analysis of biological values and machine learning metrics[¹⁴]. If necessary, we selectively incorporate amino acid sequence's biophysical properties such as hydrophobicity, charge, or volume into our deep learning analysis method to optimize the screen analysis of antibodies for a given epitope for highly-throughput and efficient antibody analysis.

Specific Application 3. Identify antibody therapeutics with optimal drug-like properties through an automated and multimodal screening method.

In-Silico Screening for identification of antibodies with drug-like properties.[⁸]. We have successfully developed an in-silico screening method to identify the desirable antibodies while filtering out the undesirable antibodies. With current advances in computational forecasts, a number of parameters including viscosity, clearance, solubility, stability, and immunogenicity are used as the guideline for in-silico screening to select the best-in-class antibody lead candidates. Although the predicted sequences are not tested in wet-lab yet, we characterized the VAEResTL-generated CDR-H3 sequences compared to seed CDR-H3 sequences on a number of therapeutic indexes through in-silico screening (FIG. 27, panels A-F). In order to screen the CDR-H3 sequences' viscosity and clearance, we measured net-charge and hydrophobicity (H) by calculating every amino acid sequence of the CDR-H3 sequences. For all the sequences in the library the net-charge is calculated at a given pH=7.0 and the hydrophobicity scale used is “eisenberg”. The optimal net-charge for drug clearance is between 0-6.2 with H of <0. Therefore, we filtered out the sequences with a net-charge of <0 (FIG. 27, panel A, marked with red box) and a H of <0 (FIG. 27, panel B, marked with red box). We also calculated the stability of CDR-H3 sequences based on proteins and their dipeptide composition and filtered out sequences with stability values of >40 and <20 (FIG. 27, panel C, marked with red boxes). We then ran VAEResTL-generated CDR-H3 amino acid sequences through CamSol to estimate their solubility. We filtered out sequences with the CamSol score of <0.2 (FIG. 27, panel D, marked with red box) according to Sormanni et al., guidelines. The low immunogenicity of antibodies/nanobodies is an essential biophysical property for their therapeutics developability. We predicted the peptide binding affinity of all padded CDR-H3 sequences to MHC Class II by utilizing NetMHCIIpan to reduce their immunogenicity. We then used peptide's % Rank of predicted affinity that we calculated when comparing CDR-H3 sequences with a set of 200,000 random natural peptides. We predicted affinity for a set of 26 HLA alleles which covers over 98% of the global population. We filtered out the sequences with a % Rank of <2.5 (FIG. 10E, marked with red box) and % Rank of <70 (FIG. 27, panel F, marked with red box) (SARS-CoV-2 average % Rank). After employing in-silico screening anti-body/nanobody CDR-H3 variants with desired viscosity, clearance, solubility, stability, and immunogenicity remained as potential lead candidates.

Through the in-silico screening, we identified antibody paratope residue CDR-H3 sequence variants with optimized multi-parameters that can be further evaluated in a wet-lab setting. The results of our in-silico analysis method set the stage for developing an automated and multimodal screening approach for a faster, more efficient, and more accurate antibody screening.

In the antibody discovery process, millions of antibodies are evaluated for many biophysical and therapeutic properties to find the top few with the highest potential for being possibly effective drugs. The final candidates are selected through a series of tests that assess how potent they are, how they work, and how easy they are to manufacture by rigorously measuring many biophysical and therapeutic properties. These complex processes are lengthy, costly, and inefficient through conventional antibody discovery and wet-lab experimentations. In contrast, an automated in-silico and multimodal screening approach can identify antibody paratopes with drug-like properties rapidly and efficiently. To achieve this, we develop an ensemble model by utilizing the biological and key therapeutic property values offered by our deep learning model to identify a shortlist of antibody therapeutics with optimal drug-like properties. Our model is based on an automated and multimodal screening approach to identify novel drug candidates. Our multimodal screening approach has the capacity to simultaneously capture the total biophysical and therapeutic properties space to select the best antibody lead candidates faster. We screen the generated anti-HER2 antibody paratopes and their predicted 3D structures by our proposed model to identify the best-in-class antibodies for breast cancer treatment. We formulate developability and simulation methods to assess and evaluate the selected antibodies with optimal drug-like properties in-silico.

Using methods and systems of the present disclosure, we demonstrate the ability to develop deep learning models for antibody-antigen binding interaction studies. We illustrate comprehensive computational antibody generation and analysis tools for contact prediction and novel antibody paratope generation and achieve results on anti-HER2 antibodies with identified MOAs. The results establish a solid deep learning-based model for rapid and accurate antibody design and analysis by generating new antibody paratopes and predicting their 3D structures against novel or existing targets. These studies allow us to gain a more in-depth understanding of how to design novel, effective, and safe antibody therapeutics rapidly and accurately. We have gathered sufficient preliminary results and developed the expertise to ensure that the studies proposed here are highly feasible to accomplish. This research has a significant impact on our unique ability to design and discover antibody therapeutics more importantly for novel and difficult targets and for unmet therapeutic needs that are struggling to be fulfilled. The disclosed technologies dramatically cut the antibody therapeutics discovery and development time, cost, and risks from the very early stage of the process and increase the development success rate of effective and safe antibody therapeutics for cancer treatment.

This study is extended to evaluate the scalability of the deep learning paratope generation and 3D structure prediction model on other breast cancer targets. The deep learning algorithms are expanded to leverage the antibody specificity and affinity with selective validation approaches. We further validate the novel antibodies designed and generated by its deep learning-based platform through future in-house in-vitro validation.

### REFERENCES

[1] Lu, R M., Hwang, Y C., Liu, I J. et al. Development of therapeutic antibodies for the treatment of diseases. J Biomed Sci 27, 1 (2020). https://doi.org/10.1186/s12929-019-0592-z is incorporated by reference in its entirety.
[2] Obrezanova, Olga et al. “Aggregation risk prediction for antibodies and its application to biotherapeutic development.” mAbs vol. 7, 2 (2015): 352-63. doi:10.1080/19420862.2015.1007828 is incorporated by reference in its entirety.
[3] Sankar, K., Hoi, K. H., Yin, Y., Ramachandran, P., Andersen, N., Hilderbrand, A., McDonald, P., Spiess, C., & Zhang, Q. (2018). Prediction of methionine oxidation risk in monoclonal antibodies using a machine learning method. mAbs, 10(8), 1281-1290. https://doi.org/10.1080/19420862.2018.1518887 is incorporated by reference in its entirety.
[4] Vanessa Isabell Kurtz, Alexander Rosenberg Johansen, Morten Nielsen, Jose Juan Almagro Armenteros, Henrik Nielsen, Casper Kaae Sonderby, Ole Winther, Soren Kaae Sonderby, An introduction to deep learning on biological sequence data: examples and solutions, Bioinformatics, Volume 33, Issue 22, 15 Nov. 2017, Pages 3685-3690, https://doi.org/10.1093/bioinformatics/btx531 is incorporated by reference in its entirety.
[5] Richard A Norman, Francesco Ambrosetti, Alexandre M J J Bonvin, Lucy J Colwell, Sebastian Kelm, Sandeep Kumar, Konrad Krawczyk, Computational approaches to therapeutic antibody design: established methods and emerging trends, Briefings in Bioinformatics, Volume 21, Issue 5, September 2020, Pages 1549-1567, https://doi.org/10.1093/bib/bbz095 is incorporated by reference in its entirety.
[6] Ge Liu, Haoyang Zeng, Jonas Mueller, Brandon Carter, Ziheng Wang, Jonas Schilz, Geraldine Horny, Michael E Birnbaum, Stefan Ewert, David K Gifford, Antibody complementarity determining region design using high-capacity machine learning, Bioinformatics, Volume 36, Issue 7, 1 Apr. 2020, Pages 2126-2133, https://doi.org/10.1093/bioinformatics/btz895 is incorporated by reference in its entirety.
[7] Graves J, Byerly J, Priego E, Makkapati N, Parish S V, Medellin B, Berrondo M. A Review of Deep Learning Methods for Antibodies. Antibodies (Basel). 2020 Apr. 28; 9(2):12. doi: 10.3390/antib9020012. PMID: 32354020; PMCID: PMC7344881 is incorporated by reference in its entirety.
[8] Khalilian, S.; Moti, Z.; Baloochestani, A.; Hallaj, Y.; Chavosh, A. and Hemmatian, Z. (2022). VAEResTL: A Novel Generative Model for Designing Complementarity Determining Region of Antibody for SARS-CoV-2. Proceedings of the 15th International Joint Conference on Biomedical Engineering Systems and Technologies—Volume 3: BIOINFORMATICS, 2022, pages 109-116, ISBN 978-989-758-552-4, DOI: 10.5220/0010823700003123 ISSN 2184-4305 is incorporated by reference in its entirety.
[9] Raphael J. L. Townshend, Rishi Bedi, Patricia A. Suriana, and Ron O. Dror. 2019. End-to-end learning on 3D protein structure for interface prediction. Proceedings of the 33rd International Conference on Neural Information Processing Systems. Curran Associates Inc., Red Hook, NY, USA, Article 1401, 15642-15651 is incorporated by reference in its entirety.
[10] Jin, W., Wohlwend, J., Barzilay, R., and Jaakkola, T. Iterative refinement graph neural network for anti-body sequence-structure co-design. arXiv preprint arXiv:2110.04624, 2021 is incorporated by reference in its entirety.
[11] Ingraham, J., Garg, V. K., Barzilay, R., and Jaakkola, T. Generative models for graph-based protein design. Neural Information Processing Systems, 2019 is incorporated by reference in its entirety.
[12] Matthew I. J. Raybould, Claire Marks, Konrad Krawczyk, Bruck Taddese, Jaroslaw Nowak, Alan P. Lewis, Alexander Bujotzek, Jiye Shi, and Charlotte M. Deane. Five computational developability guidelines for therapeutic antibody profiling, Proc Natl Acad Sci USA. 2019 Mar. 5; 116(10):4025-4030. doi: 10.1073/pnas.1810576116. Epub 2019 Feb 14. PMID: 30765520; PMCID: PMC6410772 is incorporated by reference in its entirety.
[13] Mason D M, Friedensohn S, Weber C R, Jordi C, Wagner B, Meng S M, Ehling R A, Bonati L, Dahinden J, Gainza P, Correia B E, Reddy S T. Optimization of therapeutic antibodies by predicting antigen specificity from antibody sequence via deep learning. Nat Biomed Eng. 2021 June; 5(6):600-612. doi: 10.1038/s41551-021-00699-9. Epub 2021 Apr 15. PMID: 33859386 is incorporated by reference in its entirety.
[14] Khalilian, S., Nasr I. M., Moti, Z., Baloochestani, A., Chavosh, A. and Hemmatian, Z.” A Deep Dimensionality Reduction method based on Variational Autoencoder for Antibody Complementarity Determining Region Sequences Analysis.” Proceedings of the 14th International Conference on Bioinformatics and Computational Biology (BICOB), 2022, Volume 83, pages 116-125, DOI 10.29007/x25c, ISNN 2398-7340 is incorporated by reference in its entirety.
[15] Yan, Y., Tao, H., He, J., and Huang, S.-Y. The hdock server for integrated protein-protein docking. Nature protocols, 15(5):1829-1852, 2020 is incorporated by reference in its entirety.
[16] Ganea, O.-E., Huang, X., Bunne, C., Bian, Y., Barzilay, R., Jaakkola, T., and Krause, A. Independent SE(3)-equivariant models for end-to-end rigid protein docking. arXiv preprint arXiv:2111.07786, 2021 is incorporated by reference in its entirety.
[17] Jin, W., Wohlwend, J., Barzilay, R., and Jaakkola, T. Iterative refinement graph neural network for anti-body sequence-structure co-design. arXiv preprint arXiv:2110.04624, 2021 is incorporated by reference in its entirety.
[18] Ingraham, J., Riesselman, A., Sander, C., and Marks, D. Learning protein structure with a differentiable simulator. In International Conference on Learning Representations, 2018 is incorporated by reference in its entirety.
[19] Shin, J.-E., Riesselman, A. J., Kollasch, A. W., McMahon, C., Simon, E., Sander, C., Manglik, A., Kruse, A. C., and Marks, D. S. Protein design and variant prediction using autoregressive generative models. Nature communications, 12(1):1-11, 2021 is incorporated by reference in its entirety.
[20] Saka, K., Kakuzaki, T., Metsugi, S., Kashiwagi, D., Yoshida, K., Wada, M., Tsunoda, H., and Teramoto, R. Antibody design using lstm based deep generative model from phage display library for affinity maturation. Scientific reports, 11(1):1-13, 2021 is incorporated by reference in its entirety.
[21] Satorras, V. G., Hoogeboom, E., and Welling, M. E(n) equivariant graph neural networks. arXiv preprint arXiv:2102.09844, 2021 is incorporated by reference in its entirety.
[22] Costa, R. L. B., Czerniecki, B. J. Clinical development of immunotherapies for HER2+ breast cancer: a review of HER2-directed monoclonal antibodies and beyond. npj Breast Cancer 6, 10 (2020). https://doi.org/10.1038/s41523-020-0153-3 is incorporated by reference in its entirety.
[23] Pedersen M W, Jacobsen H J, Koefoed K, Dahlman A, Kjor I, Poulsen T T, Meijer P J, Nielsen L S, Horak I D, Lantto J, Kragh M. Targeting Three Distinct HER2 Domains with a Recombinant Antibody Mixture Overcomes Trastuzumab Resistance. Mol Cancer Ther. 2015 March; 14(3):669-80. doi: 10.1158/1535-7163.MCT-14-0697. Epub 2015 Jan 22. PMID: 25612619 is incorporated by reference in its entirety.
[24] Richard S, Selle F, Lotz J P, Khalil A, Gligorov J, Soares D G. Pertuzumab and trastuzumab: the rationale way to synergy. An Acad Bras Cienc. 2016; 88 Suppl 1:565-77. doi: 10.1590/0001-3765201620150178. PMID: 27275646 is incorporated by reference in its entirety.
[25] Mohammadi M, Jeddi-Tehrani M, Golsaz-Shirazi F, Arjmand M, Bahadori T, Judaki M A, Shiravi F, Zare H A, Haghighat F N, Mobini M, Amiri M M, Shokri F. A Novel Anti-HER2 Bispecific Antibody With Potent Tumor Inhibitory Effects In Vitro and In Vivo. Front Immunol. 2021 Feb. 17; 11:600883. doi: 10.3389/fimmu.2020.600883. PMID: 33679691; PMCID: PMC7927792 is incorporated by reference in its entirety.
[26] Capelan M, Pugliano L, De Azambuja E, Bozovic I, Saini K S, Sotiriou C, Loi S, Piccart-Gebhart M J. Pertuzumab: new hope for patients with HER2-positive breast cancer. Ann Oncol. 2013 February; 24(2):273-282. doi: 10.1093/annonc/mds328. Epub 2012 Aug. 21. PMID: 22910839 is incorporated by reference in its entirety.

### Example 7: Artificial Intelligence for Humanized Antibody Therapeutics Design

Antibody (Ab) therapeutics are becoming an essential class of biologics used to treat known and novel diseases due to their unique specificity and efficacy. However, the long time and the high cost associated with developing therapeutic antibodies may present key challenges in the availability of antibody therapeutics despite the increasing need for these therapies. Moreover, given the massive antibody space and the other methods' limitations, numerous antibody mutations may yet to be discovered for known or novel diseases. The present disclosure provides in-silico antibody library design methods offer fast, efficient, accurate, and low-cost discovery approaches to boost the speed of antibody discovery while exploring a massive antibody space that is not feasible through the other approaches.

In antibody therapeutics, the complementarity determining regions (CDRs) of the variable region residue (Fv) play a significant role in antibody specificity and antibody binding to a target. However, besides CDRs, other fractions of Fv region, including framework regions (FRs), play a substantial role in determining the therapeutic properties of antibodies. For example, mutations in the FRs may lead to a shifted CDR, which can impact antibody binding ability. Furthermore, FRs' mutations may lead to better antibody binding by decreasing the entropy and antibody biochemical properties such as stability, charge, and hydrophobicity. Other in-silico library generation approaches may focus merely on designing antibodies' CDRs, specifically the CDR3 of heavy chains (CDR-H3), and cannot design the entire variable regions. Furthermore, such approaches may not humanize the Fv region of the designed antibodies to alleviate unwanted therapeutic properties, which may trigger immune responses that can lead to antidrug antibody formation. Thus, the rapid design and humanization of antibodies' Fv, composed of seven amino-acid regions, in which four are the FRs, and three are the CDRs, is of critical importance. Using methods and systems of the present disclosure, a deep learning-based model was developed to design new antibody sequences against a novel antigenic target. The deep learning model offers significant advantages over other models, such as its unique ability to rapidly and accurately design CDR-H3 sequences when the input data is scarce for a novel or existing target.

Using methods and systems of the present disclosure, new antibodies against SARS-CoV-2 were designed, and an antibody CDR-H3 hit-generation tool was developed. Further, the extracted key features offered by the deep learning model were utilized to analyze antibody sequences, and successfully demonstrated the Ab's specificity and cross-reactivity to one or multiple targets.

Using methods and systems of the present disclosure, a computational model is developed to design and humanize antibody Fvs of the heavy chain (VH) and light chain (VL) for novel and/or difficult targets (e.g., SARS-CoV-2). The scalability of the computational model is demonstrated in designing humanized antibodies against diverse targets. Biological values of the humanized antibody Fvs predicted by the computational model are assessed.

Humanized antibody variable region residues (Fvs), including CDRs and FRs, may be accurately designed, in order to discover the best-humanized antibody therapeutic hits with drug-like properties through a fast, high-throughput, and precise computational antibody design methodology. Such approaches may use a generative model that can learn the transformative relationships of antibody sequences directly from the complex and high-dimensional amino-acid sequences space. The generative model can design new and highly diverse humanized sequence variants with improved physicochemical properties. A few baseline models are adopted to evaluate the performance of the model compared to the baseline models. Evaluation metrics of the predicted sequences, including accuracy, sensitivity, and specificity, are determined to assess the biologically valuable predicted humanized antibodies V_{H} and V_{L}. To increase the scalability of the AI engine, the deep generative model is trained using antibody sequences against various antigens.

The humanized antibody therapeutics design may be summarized as follows.
1. Acquire antibody sequence datasets.
2. Develop and train a deep generative model to design and humanize antibody Fvs.
3. Design an automated AI engine that designs humanized antibody Fvs sequences, including FRs, and CDRs for both heavy chain (VH) and light chain (V_{L}).
4. Evaluate the deep generative model's performance.
5. Assess the quality of the designed humanized antibody sequences of the VH and the VL.

The availability of massive training data may constrain progress in deep generative and computational design. However, using systems and methods of the present disclosure, biologically valuable antibody CDR-H3 sequences against SARS-CoV-2 were demonstrated, despite training data shortage.

With the above capabilities, we can accurately design and humanize antibody variable regions for novel and difficult targets such as SARS-CoV-2, which can enhance the rapid response ability against a future pandemic. Moreover, the AI-based tool is capable of discovering therapeutic antibodies against various targets (e.g., disease targets such as cancer targets). Therefore, development of effective and safe therapeutic antibodies for single antibody therapy, combination antibody therapy, and the design of next-generation antibody therapeutics may be considerably enhanced.

### Example 8: Artificial Intelligence Epitope Mapping for Antibody-Drug Design

Antibodies can specifically target an antigen or a damaged cell and bind to it, which results in improving the efficacy of antibody therapeutics profile for cancer treatments. An important task in antibody therapeutics discovery is to rapidly identify whether an antibody can recognize a specific epitope on an antigen surface and bind to it. Characterization of antibody-paratope:antigen-epitope interaction provides valuable mechanistic insights that can enhance antibody therapeutics' effectiveness. However, other methods may be overly costly and labor-intensive as repetitive wet-lab experimentations are required to sufficiently explore the antibody sequence space. Moreover, due to the extended time and the significant cost associated with other methods, such analysis may be used at the late stage of the discovery and development process and on a limited number of antibody candidates or antigenic variants. This reduces the number of goal shots among the antibody sequences galaxy and increases the risk of failure.

Using methods and systems of the present disclosure, an early stage, high-throughput, and efficient method is enabled for rapid and accurate antibody analysis, which relies on only analysis of input amino acid sequences. A deep learning-based model was developed for characterization and modeling of the antibody-paratope:antigen-epitope binding interactions for epitope prediction and identifying antibodies' mechanisms of action. By characterizing the antibody-antigen binding interactions, the deep learning model is capable of grouping the antibodies with a similar mode of recognition associated with the antigen binding region and superbly localize the hot spot residues.

Further, a ranking model was developed to identify the best matches in the antibodies' competition to bind to a specific epitope on a given antigen. Overexpression of human epidermal growth factor receptor (HER2) may be present in breast cancer patients, and anti-HER2 antibodies may have robust anti-tumor activity in patients with HER2-overexpressing breast cancer. Moreover, in addition to the extensive availability of anti-HER2 antibody data against HER2 variant epitope domains, the HER2 receptor comprises variant epitope domains that have binding interactions with various antibodies leading to different MOAs. As a result, the interaction between anti-HER2 antibodies and HER2 receptor can serve as an excellent model for developing a deep learning-based epitope prediction model for breast cancer treatment. Using methods and systems of the present disclosure, an antibody hit generation tool is developed, which provides a large library of antibody mutations against a specific target to extend existing anti-HER2 databases and train a deep learning-based epitope prediction model. Validation analysis is performed on the HER2 predicted epitopes using an analysis tool of the present disclosure to analyze the predicted epitopes based on their specificity and cross-reactivity.

The design of effective and safe antibody therapeutics may rely on an accurate mechanism of action identification through precise epitope-paratope interaction mapping. Using methods and systems of the present disclosure, deep learning-based models were developed to analyze antibody binding to a given antigen to enhance the epitope prediction early in the antibody discovery process. The deep learning-based models can identify and group antibodies based on their binding preference to a specific epitope on the antigen surface using only amino acid sequence data. By enabling a more precise and rich analysis compared to wet-lab experimentation approaches and computational structural studies, methods and systems of the present disclosure enable a rapid and low-cost antibody analysis tool for the discovery and development of antibody therapeutics to treat breast cancer.

Antibody therapeutics have increasingly shown fewer adverse effects because of their unique capability to selectively recognize a specific epitope of an antigenic target and bind to it. An epitope is the specific region of an antigen to which an antibody binds through its binding site called a paratope. Antibodies' multifaceted properties through their interactions with specific epitopes make them an important modality in cancer treatment. For example, antibodies against Human epidermal growth factor receptor 2 (HER2) may be utilized for breast cancer treatment. However, different antibodies may have binding to different epitopes on HER2, leading to different mechanisms of action (MOA). The MOA for antibodies against a given antigen may be unknown in the early discovery stage. Therefore, to increase the effectiveness of systemic treatment through targeted therapy, accurate epitope prediction for determining the binding of an antibody to a specific epitope of the given antigen is an essential task in the antibody lead identification stage of the antibody therapeutics discovery process.

Accurate epitope prediction provides important mechanistic insights that can also enhance an antibody drug's safety. The identified MOA can also help design antibody therapeutics for combination therapies. Accurate epitope prediction may also help design the next generation of antibody therapeutics, such as bispecific and trispecific antibodies, and may result in strong survival rates among cancer patients. Other technologies for epitope mapping may be costly, laborious, and time-consuming, as they may require repetitive testing of many mutants to identify an antibody that binds to a specific epitope on a given antigen. Yet, these methods may not offer rich and as nearly strong information. In parallel to other experimentation methods, in-silico methods can be performed to rapidly predict molecular attributes for epitope mapping purposes, providing great potential for accelerating antibody discovery. Other computational methods may require antibody structural data; however, at the early discovery, the antibodies can only be specified by their sequences.

Recognizing the above needs, using systems and methods of the present disclosure, an in-silico model is developed can accurately and rapidly map epitopes using only the amino acid sequences. This can accelerate the development of antibody therapeutics, especially at the early stages of discovery, to mitigate the risks associated with the development of antibody therapeutics. A deep learning-based model may be developed to design new antibody sequences against a novel antigenic target. This deep learning model offers significant advantages over other models, such as its unique ability to rapidly and accurately design the third complementarity determining regions in the heavy chain (CDR-H3) sequences when the input data is scarce for a novel or existing target.

Using systems and methods of the present disclosure, a deep learning model is developed for rapid and accurate epitope prediction based on only the amino acid sequences, in order to elucidate the antibodies' MOA for accurate lead identification and for optimizing the safety and efficacy of antibody therapeutics. An example focus is on a disease target such as a cancer (e.g., breast cancer) target.

First, an AI-based algorithm is developed for accurate and fast epitope mapping. A conditional deep learning-based model is developed to analyze the interaction between antibodies' paratope with antigens' epitopes for accurate epitope mapping. The deep learning model for rapid and accurate epitope prediction is trained using publicly available antibody data in combination with antibody data against HER2 that is generated by a deep generative model of the present disclosure. A few baseline models are adopted to evaluate the performance of the deep learning model, and evaluation metrics on the predicted epitopes, including accuracy, precision, and AUC, are determined. The antibody drug discovery time can be reduced by up to 1Ox or more.

Second, antibody interactions with a given epitope on an antigen surface are analyzed using a ranking method. The best antibody match for an epitope is identified. The ranking method screens the HER2 sequences paired with antibody sequences to identify antibodies that interact with specific HER2 hot-spot residues. A binding interaction index, such as minimum residue score, is incorporated to optimize the ranking model. For example, the epitope prediction may be obtained with at least 60%, 65%, 70%, 75%, 80%, 85%, 90%, 95%, 96%, 97%, 98%, or 99% accuracy, sensitivity, specificity, positive predictive value, negative predictive value, or a combination thereof. Amino acids' physicochemical attributes, including hydrophobicity, polarity, exposed surface, and accessibility, are used to assess the predicted epitopes. The predicted epitopes are validated based on their specificity and cross-reactivity.

The AI-based model is used to rapidly analyze antibody candidates against novel or existing targets using accurate epitope mapping to identify the lead antibody candidates with low cost and runtime. This tool is used for the discovery and development of antibody therapeutics to treat breast cancer. The developed deep learning models are scalable toward other breast cancer targets to identify antibodies' MOA, which leads to accurate antibody lead identification towards designing new antibody therapeutics for the treatment of breast cancer.

Antibodies are identified as proteins that could recognize specific antigens at high specificity and affinity and bind to them through their binding site called paratope. The specific area on the antigen recognized by the antibody paratope is called the epitope. The information about the binding of antigen epitope to antibody paratope can provide imperative mechanistic intuitions indicating for what application an antibody might be useful. Thus, accurate localization of the binding region between antigen epitope and antibody paratope may be critically important for effective and safe antibody therapeutics design through an identified mechanism of action (MOA). Moreover, antibodies hold a diverse set of clinically relevant mechanisms of action, which makes them superior therapeutic candidates that can treat a wide range of diseases, including cancer. Therefore epitope: paratope analysis at the early stage of antibody therapeutics discovery is essential to distinguish antibodies' mode of recognition, which in return can identify their MOA. The early-stage epitope prediction increases the success rate of effective and safe antibody therapeutics design and reduces the risk of costly late-stage failure.

Epitope prediction techniques may be developed to characterize the epitope: paratope interactions. Wet-lab experimentation methods may require repetitive testing of many mutants, making them costly, laborious, and time-consuming for identifying an antibody that binds to a specific epitope on a given antigen. In parallel to other experimentation methods, in-silico methods can rapidly predict molecular attributes for epitope prediction purposes, providing great potential for accelerating antibody discovery. Computational methods may be based on structural studies; however, at the early discovery stage, the antibodies can only be indicated by their sequences. Hence, the development of antibody therapeutics can be expedited through an in-silico model that can accurately and rapidly characterize epitope: paratope interactions using only the amino acid sequences.

Using methods and systems of the present disclosure, deep learning technologies are developed that can provide rapid, high-resolution, and efficient epitope: paratope interaction analysis. By characterizing the antibody-antigen binding interactions, the deep learning model groups the antibodies with a similar mode of recognition associated with the antigen binding region and superbly localize the hot spot residues. A ranking model is developed to identify the best matches in the antibodies' competition to bind to a specific epitope on the given antigen. Anti-HER2 antibodies serve as an excellent model for developing the deep learning epitope prediction model, with the extensive availability of anti-HER2 antibody data in combination with our proprietary in-silico-designed anti-HER2 antibody data and existing antigen epitope variant domains of the HER2 receptor.

Antibodies retain a diverse set of clinically significant mechanisms of action (MOA) that may be rigorously studied at the earliest stage of the antibody therapeutics discovery process. Therefore, there is an urgent need for accurate, fast, and scalable technologies in antibody analysis and epitope prediction. In-silico methods can rapidly predict molecular attributes for epitope prediction purposes; however, these methods may require antibody structural data, while at the early discovery stage, the antibodies can be only specified by their sequences. Thus, in-silico technologies that can accurately and rapidly map epitopes using only the amino acid sequences can accelerate the development of antibody therapeutics to mitigate the risks associated with antibody therapeutics development at the very early stage of the discovery process.

Deep learning models may be integrated for antibody-antigen interaction studies. Other in-silico models may not be able to predict the MOA through the epitope prediction at the earliest stage of the antibody discovery process as they mainly require structural data through expensive and time-consuming crystallography methods, which may lead to a longer and more expensive drug development process. However, a deep learning technology relying on only the amino acid sequence data may provide fast and accurate insights into the antibody-antigen interactions using sequence data at the very early stage of the antibody discovery process.

Antibodies' amino acid sequences may be modeled to test the antibody paratope-antigen epitope interactions. In contrast to other computational models, the model of the present disclosure models the antibodies' paratope amino acid sequences and antigens' epitope amino acid sequences to study their binding interaction faster and more accurately.

The model may utilize antibody mutations in the binding modeling for the most efficient prediction of the best antibody match to a given epitope. The in-silico approach may be armed with a ranking model while screening the possible binding sites between antibodies and different epitopes of a specific antigen. The ranking model selects among antibodies interacting with the epitope hot spot residues to best support the epitope prediction and find the best-paired epitope-paratope matches. This provides highly efficient MOA prediction through epitope mapping and identification of the antibody lead candidates.

Systematic analysis of antibody paratope-antigen epitope interaction competition may be performed. Evaluation metrics, such as accuracy, specificity, and sensitivity, are used to validate the deep learning and ranking models. While screening antibody competition with respect to a specific antigen epitope competition index, biological, and machine learning metrics are combined to better characterize and map the epitopes.

Using methods and systems of the present disclosure, anti-HER2 antibodies and HER2 receptor interactions are modeled using a deep learning-based model. Overexpression of human epidermal growth factor receptor (HER2) may be present in breast cancer patients, and anti-HER2 antibodies may have robust anti-tumor activity in patients with HER2-overexpressing breast cancer. Moreover, in addition to the extensive availability of anti-HER2 antibody data against HER2 variant epitope domains, HER2 receptor comprises variant epitope domains that have binding interactions with various antibodies leading to different MOAs. As a result, the interaction between anti-HER2 antibodies and HER2 receptor can serve as an excellent model for developing the deep learning-based epitope prediction model for breast cancer treatment.

Complementary approaches are pursued for the systematic and efficient analysis of antibody paratope-antigen epitope interactions. Different machine learning and deep learning models are developed, and trained using natural and designed antibody sequences to achieve a model with high scalability (FIG. 28).

A deep learning-based model, VAEResTL, was developed to design new antibody sequences and predict antibody mutations specific to a novel antigenic target (FIG. 29).

The AI-based algorithm is capable of accurate and fast epitope mapping. The systems and methods of the present disclosure are used to rapidly and accurately design the antibody sequences of the third complementarity determining regions in the heavy chain (CDR-H3) against a novel target when the input data is scarce.

Three baseline models' generated sequences were compared with the generated sequences from a deep learning method of the present disclosure (Table 7). The overall machine learning (ML) and biophysical metrics demonstrate the deep generative model, VAEResTL, can more efficiently predict CDR-H3 sequences with binding ability to the antigenic target (Table 7) compared to the other baseline models.

TABLE 7
Baseline models comparison. Biological Characteristics and Machine
Learning Metrics for generated CDR-H3 sequences by VAERes,
VAEResTL, HMM, LSTM, and Seed sequences.

| | | | | | | Machine Learning Metrics | | | |
|---|---|---|---|---|---|---|---|---|---|
| | Biophysical Characteristics | | | | | BLEU | | | |
| Method | NW | H | Method | NW | H | (2-gram) | (3-gram) | (4-gram) | JSD |
| VAERes | [symbol] | [symbol] | [symbol] | [symbol] | [symbol] | [symbol] | [symbol] | [symbol] | [symbol] |
| VAEResTL | [symbol] | [symbol] | [symbol] | [symbol] | [symbol] | [symbol] | [symbol] | [symbol] | [symbol] |
| LSTM | [symbol] | [symbol] | [symbol] | [symbol] | [symbol] | [symbol] | [symbol] | [symbol] | [symbol] |
| HMM | [symbol] | [symbol] | [symbol] | [symbol] | [symbol] | [symbol] | [symbol] | [symbol] | [symbol] |
| Seed | [symbol] | [symbol] | [symbol] | [symbol] | [symbol] | [symbol] | [symbol] | [symbol] | [symbol] |

[symbol]

indicates data missing or illegible when filed

The biophysical properties of designed CDR-H3 sequences were compared to the biological CDR-H3 sequences, and the accuracy of the model was validated in designing novel antibody sequences with valuable properties (FIG. 30). It was found from the ML, visualization heatmaps (FIG. 31, panels A1-C1) and logo plots (FIG. 31, panels A2-C2) that although VAEResTL changed certain CDR-H3 amino acid positions and their frequency distributions, the overall pattern of VAEResTL-generated sequences are closer to seed sequences as compared to the VAERes-generated CDR-H3 sequences (FIG. 31). With current advances in computational forecasts, a number of parameters including viscosity, clearance, solubility, stability, and immunogenicity are used as the guideline for in-silico screening to select the best-in-class antibody lead candidates. Although the predicted sequences are not tested in wet-lab yet, the VAEResTL-generated CDR-H3 sequences were characterized as compared to seed CDR-H3 sequences on a number of therapeutic indexes through in-silico screening (FIG. 32). Through the in-silico screening, CDR-H3 sequence variants were identified with optimized multi-parameters that can be further evaluated in a wet-lab setting.

The deep generative model was tested across various antigenic targets, and the scalability of the model was demonstrated on antibody sequence design against a specific target. The deep-learning design model provides a large library of possible CDR-H3 mutations that can have binding specificity to different epitopes of a given antigen target. Importantly, the antibody mutations designed by the model can extend the epitope mapping to study the antibodies' competition with respect to appropriate epitope residues on the antigen surface.

Antibody mutations that have binding specificity to a specific epitope among various epitopes of an antigenic target are used. Systems and methods of the present disclosure are used to accurately find antibodies and the corresponding antigen residues that contribute to a binding interaction between antibody paratope and antigen epitope leading to a specific MOA using only amino acid sequences. Deep learning models are developed and trained to characterize the epitope-paratope interactions for epitope prediction purposes (FIG. 28). A focus is on HER2 receptor on the surface of the HER2-expressing breast cancer cells and anti-HER2 antibodies that have defined binding labels to specific epitopes of a given antigen while developing the deep learning model. For example, antibodies that bind to various domains of HER2 receptor are analyzed.

The model is tested on a disease (e.g., cancer such as breast cancer) antigenic target and appropriate antibodies that have binding to this antigenic target. All possible mutations are tested across a large library of antibody amino acid sequences to identify antibody sequences that interact with a specific epitopes on the HER2 surface. A deep learning-based model is developed in which a specific antigen epitope is conditioned to test antibody paratope interactions against the given antigen epitope. With this approach, we can efficiently extract the key features related to the binding specificity of antibody sequences to given specific residues of the antigenic target and identify the best match leading to a specific signaling pathway. The model uses amino acid sequences of HER2 epitopes and anti-HER2 antibodies paratope to model antibody-antigen interactions. The results of the deep learning model enhance the competition analysis between antibody paratope-antigen epitope pairs. A few baseline models are adopted to evaluate the performance of the model and compare with the baseline models. Accuracy, precision, and AUC are used to compare the performance of the deep learning model with the baseline models for the best epitope prediction outcomes.

In case sufficient hot-spot residues identification for each antibody sequence is not obtained, the training data may be extended from antibody CDR-H3 sequences to antibody variable region sequences. In case low consistency is obtained with the epitope prediction across different antigenic targets, then amino acids' biological properties, such as hydrophobicity, volume, and charge, may be selectively incorporated into the deep learning model for highly efficient epitope prediction across various antigenic targets.

Systems and methods of the present disclosure are used to analyze antibody interactions with a given epitope on an antigen surface using a ranking method. The deep learning model may be used for analysis of antibody sequences. Key features were extracted using the deep learning model, which is a dimensionality reduction (DR) approach referred to as VAEResDR (FIG. 33), to analyze antibody sequences. Antibodies' specificity and cross-reactivity to one or multiple targets was demonstrated for lead identification purposes.

The deep learning model was separately trained on three and four antigenic targets, respectively, by using antibodies including Trastuzumab, Ranibizumab, yeast display scFV display scFV, and OVA. Visualization plots (FIG. 34) and clustering quality metrics (Table 8) were used to evaluate the impact of the VAEResDR model on antibody amino acid CDR-H3 sequences' DR and clustering as compared to baseline methods. The 2D visualization plots demonstrate the deep learning analysis tool is capable of well-separating antibody sequence groups with valuable biophysical parameters recognized within the two-dimensional sequences space (FIG. 34, panels G and N). Both clustering models of K-Means and GMM were tested on the extracted features by the VAEResDR compared to baseline models. It was demonstrated that the deep learning analysis tool of VAEResDR outperformed PCA, t-SNE, UMAP, and deep learning methods of VAE, scCCESS (AE), and the VAEDR model (Table 8) in both five cluster and six cluster experiments.

TABLE 8
Clustering Results: Quality Metrics comparison for K-Means and GMM on
CDR-H3 sequences on PCA, t-SNE, UMPA, VAE, scCCESS (AE), VAEDR
and VAEResDR for three targets, 5 clusters and four targets, 6 clusters.

| | Clustering | 5 Cluster | | | | | 6 Cluster | | | | |
|---|---|---|---|---|---|---|---|---|---|---|---|
| Method | Algorithms | ARI | MI | HS | CS | VMS | ARI | MI | HS | CS | VMS |
| PCA | K-Means | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| | GMM | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| t-SNE | K-Means | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| | GMM | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| UMAP | K-Means | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| | GMM | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| VAE | K-Means | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| | GMM | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| scCCESS | K-Means | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| (AE) | GMM | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| VAEDR | K-Means | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| | GMM | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| VAEResDR | K-Means | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |
| | GMM | 0. [symbol] | 0. [symbol] | 0. [symbol] | 0. [symbol] | 0. [symbol] | 0. [symbol] | 0. [symbol] | 0. [symbol] | 0. [symbol] | 0. [symbol] |

[symbol]

indicates data missing or illegible when filed

An analysis was performed to assess the validity of the antibody specificity and cross-reactivity to one or multiple antigenic targets using biological and machine learning metrics (FIG. 25).

The results of the antibody analysis model may suggest that antibody sequences against antigenic target (a) may have specificity to antigenic target (b). The antibody sequences analysis model may also demonstrate that sequences against antigen target (a) may have cross-reactivity with the target (b) (FIG. 25). With these analyses, we may prevent any false-positive or false-negative results when performing sequence screening for either of the targets. The quantitative analysis demonstrates that the model achieved superior DR performance and has significant generalizability for different datasets with diverse structures in the original and high-dimensional spaces. The model provides a readily usable tool for screening CDR-H3 sequences' specificity and for identifying highly specific CDR-H3 sequences that bind to one or multiple antigenic targets.

Using systems and methods of the present disclosure, an analysis model is established to accurately and efficiently analyze the predicted epitope that the conditional deep learning model provides. Every antibody mutation is screened against HER2 receptor's epitopes on its target surface, and the binding interactions are measured to further filter the analysis. A ranking model is developed based on the binding interaction of the antibodies with each epitope. The ranking model identifies the best match among the competition. A binding interaction index such as minimum residue score is incorporated into the method to optimize the ranking model. The extracted key features offered by the deep learning model are fed into the clustering model to evaluate the ranking model performance further. Visualization and statistical measures are used to assess the ranking model's performance further. The predicted epitopes are further validated based on their specificity and cross-reactivity. Amino acids' physicochemical attributes, including hydrophobicity, polarity, exposed surface, and accessibility, are used to assess the predicted epitopes.

If necessary, amino acid sequences' biophysical properties, such as hydrophobicity, charge, and volume, may be selectively incorporated into the ranking method to optimize the screen analysis of antibody interaction match to a given epitope for a highly efficient epitope prediction and epitope mapping.

Deep learning models are developed for antibody-antigen binding interaction studies with comprehensive computational analysis tools for epitope prediction resulting in anti-HER2 antibodies with identified MOA. A solid deep learning-based model is established for rapid and accurate epitope prediction against novel or existing targets. As there is a large unmet medical need for antibodies MOA identification to accurately and effectively design new antibodies against breast cancer targets, this study is extended to evaluate the scalability of the deep learning epitope prediction model on other breast cancer targets. The deep learning algorithms is expanded to leverage the antibody specificity and affinity attributes with selective validation approaches. An antibody sequence analysis tool is developed to dramatically cut the antibody therapeutics discovery and development time, cost, and risks from the very early stage of the process, and increase the development's success rate of effective and safe antibody therapeutics for cancer treatment.

### REFERENCES

[1] Lu, R M., Hwang, Y C., Liu, I J. et al. Development of therapeutic antibodies for the treatment of diseases. J Biomed Sci 27, 1 (2020). https://doi.org/10.1186/s12929-019-0592-z is incorporated by reference herein in its entirety.
[2] K. Murphy, C. Weaver, Janeway's Immunobiology, 12th ed., New York, Garland Science, 2017 is incorporated by reference herein in its entirety.
[3] Nilvebrant J, Rockberg J. An Introduction to Epitope Mapping. Methods Mol Biol. 2018; 1785:1-10. doi: 10.1007/978-1-4939-7841-0_1. PMID: 29714008 is incorporated by reference herein in its entirety.
[4] Victor H. Obungu, Valentina Gelfanova, Radhakrishnan Rathnachalam, Anna Bailey, Joanne Sloan-Lancaster, and Lihua Huang Determination of the Mechanism of Action of Anti-FasL Antibody by Epitope Mapping and Homology Modeling, Biochemistry 2009 48 (30), 7251-7260 DOI: 10.1021/bi900296g is incorporated by reference herein in its entirety.
[5] Willems A, Gauger K, Henrichs C, Harbeck N. Antibody therapy for breast cancer. Anticancer Res. 2005 May-June; 25(3A):1483-9. PMID: 16033049 is incorporated by reference herein in its entirety.
[6] Gan H K, Burgess A W, Clayton A H, Scott A M (2012) Targeting of a conformationally exposed, tumor-specific epitope of EGFR as a strategy for cancer therapy Cancer Research 72:2924-2930, https://doi.org/10.1158/0008-5472.CAN-11-3898 is incorporated by reference herein in its entirety.
[7] Koefoed K Steinaa L Soderberg J N Kjor I Jacobsen H J Meijer P J Haurum J S Jensen A Kragh M Andersen P S Pedersen M W (2011) Rational identification of an optimal antibody mixture for targeting the epidermal growth factor receptor mAbs 3:584-595. https://doi.org/10.4161/mabs.3.6.17955 is incorporated by reference herein in its entirety.
[8] David Zahavi, Louis Weiner, Monoclonal Antibodies in Cancer Therapy, Antibodies (Basel). 2020 September; 9(3): 34. Published online 2020 Jul 20. doi: 10.3390/antib9030034 is incorporated by reference herein in its entirety.
[9] Brooks B D. The importance of epitope binning for biological drug discovery. Curr Drug Discov Technol. 2014 June; 11(2):109-12. doi: 10.2174/1570163810666131124233827. PMID: 24266537; PMCID: PMC4033553 is incorporated by reference herein in its entirety.
[10] Abdiche Y N, Miles A, Eckman J, Foletti D, Van Blarcom T J, Yeung Y A, Pons J, Rajpal A. High-throughput epitope binning assays on label-free array-based biosensors can yield exquisite epitope discrimination that facilitates the selection of monoclonal antibodies with functional activity. PLoS One. 2014 Mar. 20; 9(3):e92451. doi:10.1371/journal.pone.0092451. PMID: 24651868; PMCID: PMC3961344 is incorporated by reference herein in its entirety.
[11] Garman E F. Developments in x-ray crystallographic structure determination of biological macromolecules. Science. 2014 Mar. 7; 343(6175):1102-8. doi: 10.1126/science.1247829. PMID: 24604194 is incorporated by reference herein in its entirety.
[12] Ditto N T, Brooks B D. The emerging role of biosensor-based epitope binning and mapping in antibody-based drug discovery. Expert Opin Drug Discov. 2016 October; 11(10):925-37. doi: 10.1080/17460441.2016.1229295. Epub 2016 Sep. 8. PMID: 27600831 is incorporated by reference herein in its entirety.
[13] Abbott W M, Damschroder M M, Lowe D C. Current approaches to fine mapping of antigen-antibody interactions. Immunology. 2014 August; 142(4):526-35. doi: 10.1111/imm.12284. PMID: 24635566; PMCID: PMC4107663 is incorporated by reference herein in its entirety.
[14] Abbott W M Damschroder M M Lowe D C (2014) Current approaches to fine mapping of antigen-antibody interactions Immunology 142:526-535. https://doi.org/10.1111/imm.12284 is incorporated by reference herein in its entirety.
[15] Qi, Huan et al. Antibody Binding Epitope Mapping (AbMap) of Hundred Antibodies in a Single Run, Molecular & Cellular Proteomics, 2021, Volume 20, 100059 doi.org/10.1074/mcp.RA120.002314 is incorporated by reference herein in its entirety.
[16] Susanne Stander, Laura R. Grauslund, Maria Scarselli, Nathalie Norais, and Kasper Rand Epitope Mapping of Polyclonal Antibodies by Hydrogen-Deuterium Exchange Mass Spectrometry (HDX-MS), Analytical Chemistry 2021 93 (34), 11669-11678, DOI: 10.1021/acs.analchem.1c00696 is incorporated by reference herein in its entirety.
[17] Abdiche Y N, Miles A, Eckman J, Foletti D, Van Blarcom T J, Yeung Y A, Pons J, Rajpal A. High-throughput epitope binning assays on label-free array-based biosensors can yield exquisite epitope discrimination that facilitates the selection of monoclonal antibodies with functional activity. PLoS One. 2014 Mar. 20; 9(3):e92451. doi: 10.1371/journal.pone.0092451. PMID: 24651868; PMCID: PMC3961344 is incorporated by reference herein in its entirety.
[18] Kadam, K., Sawant, S., Kulkarni-Kale, U., and Jayaraman, V. K. (2014). “Prediction of Protein Function Based on Machine Learning Methods: an Overview,” in Genomics III—Methods, Techniques and Application (Hong Kong: iConcept Press Ltd.iConcept Press), 125-162 is incorporated by reference herein in its entirety.
[19] Casey K Hua, Albert T Gacerez, Charles L Sentman, Margaret E Ackerman, Yoonjoo Choi, Chris Bailey-Kellogg (2017) Computationally-driven identification of antibody epitopes eLife 6:e29023, https://doi.org/10.7554/eLife.29023 is incorporated by reference herein in its entirety.
[20] J. Jumper, R. Evans, A. Pritzel, T. Green, M. Figurnov, O. Ronneberger, K. Tunyasuvunakool, R. Bates, A. Zidek, A. Potapenko, A. Bridgland, C. Meyer, S. A. A. Kohl, A. J. Ballard, A. Cowie, B. Romera-Paredes, S. Nikolov, R. Jain, J. Adler, T. Back, S. Petersen, D. Reiman, E. Clancy, M. Zielinski, M. Steinegger, M. Pacholska, T. Berghammer, S. Bodenstein, D. Silver, O. Vinyals, A. W. Senior, K. Kavukcuoglu, P. Kohli, D. Hassabis, Nature 2021, 596, 583-589 is incorporated by reference herein in its entirety.
[21] Abanades B, Georges G, Bujotzek A, Deane C M. ABlooper: Fast accurate antibody CDR loop structure prediction with accuracy estimation. Bioinformatics. 2022 Jan. 31; 38(7):1877-80. doi: 10.1093/bioinformatics/btac016. Epub ahead of print. PMID: 35099535; PMCID: PMC8963302 is incorporated by reference herein in its entirety.
[22] Ruffolo, Jeffrey A., Jeremias Sulam, and Jeffrey J. Gray. “Antibody structure prediction using interpretable deep learning.” Patterns 3, no. 2 (2022): 100406, https://doi.org/10.1016/j.patter.2021.100406 is incorporated by reference herein in its entirety.
[23] Soria-Guerra R E, Nieto-Gomez R, Govea-Alonso D O, Rosales-Mendoza S., An overview of bioinformatics tools for epitope prediction: implications on vaccine developmentJ Biomed Inform. 2015 February; 53:405-14. doi: 10.1016/j.jbi.2014.11.003. Epub 2014 Nov. 10 is incorporated by reference herein in its entirety.
[24] Improved antibody-specific epitope prediction using AlphaFold and AbAdapt Zichang Xu, Ana Davila, Jan Wiamowski, Shunsuke Teraguchi, Daron M. Standley Improved antibody-specific epitope prediction using AlphaFold and AbAdapt bioRxiv 2022.05.21.492907; https://doi.org/10.1101/2022.05.21.492907 is incorporated by reference herein in its entirety.
[25] Khalilian, S.; Moti, Z.; Baloochestani, A.; Hallaj, Y.; Chavosh, A. and Hemmatian, Z. VAEResTL: A Novel Generative Model for Designing Complementarity Determining Region of Antibody for SARS-CoV-2. 15th International Joint Conference on Biomedical Engineering Systems and Technologies—Volume 3: BIOINFORMATICS, 2022, ISBN 978-989-758-552-4, ISSN 2184-4305, pages 109-116 is incorporated by reference herein in its entirety.
[26] Khalilian, S., Nasr I. M., Moti, Z., Baloochestani, A., Chavosh, A. and Hemmatian, Z.” A Deep Dimensionality Reduction method based on Variational Autoencoder for Antibody Complementarity Determining Region Sequences Analysis.” 14th International Conference on Bioinformatics and Computational Biology, BICOB, 2022, Volume 83, pages 116-125 is incorporated by reference herein in its entirety.
[27] Costa, R. L. B., Czerniecki, B. J. Clinical development of immunotherapies for HER2+ breast cancer: a review of HER2-directed monoclonal antibodies and beyond. npj Breast Cancer 6, 10 (2020). https://doi.org/10.1038/s41523-020-0153-3 is incorporated by reference herein in its entirety.
[28] Pedersen M W, Jacobsen H J, Koefoed K, Dahlman A, Kjor I, Poulsen T T, Meijer P J, Nielsen L S, Horak I D, Lantto J, Kragh M. Targeting Three Distinct HER2 Domains with a Recombinant Antibody Mixture Overcomes Trastuzumab Resistance. Mol Cancer Ther. 2015 March; 14(3):669-80. doi: 10.1158/1535-7163.MCT-14-0697. Epub 2015 Jan 22. PMID: 25612619 is incorporated by reference herein in its entirety.
[29] Richard S, Selle F, Lotz J P, Khalil A, Gligorov J, Soares D G. Pertuzumab and trastuzumab: the rationale way to synergy. An Acad Bras Cienc. 2016; 88 Suppl 1:565-77. doi: 10.1590/0001-3765201620150178. PMID: 27275646 is incorporated by reference herein in its entirety.
[30] Mohammadi M, Jeddi-Tehrani M, Golsaz-Shirazi F, Arjmand M, Bahadori T, Judaki M A, Shiravi F, Zare H A, Haghighat F N, Mobini M, Amiri M M, Shokri F. A Novel Anti-HER2 Bispecific Antibody With Potent Tumor Inhibitory Effects In Vitro and In Vivo. Front Immunol. 2021 Feb. 17; 11:600883. doi: 10.3389/fimmu.2020.600883. PMID: 33679691; PMCID: PMC7927792 is incorporated by reference herein in its entirety.
[31] Capelan M, Pugliano L, De Azambuja E, Bozovic I, Saini K S, Sotiriou C, Loi S, Piccart-Gebhart M J. Pertuzumab: new hope for patients with HER2-positive breast cancer. Ann Oncol. 2013 February; 24(2):273-282. doi: 10.1093/annonc/mds328. Epub 2012 Aug. 21. PMID: 22910839 is incorporated by reference herein in its entirety.

### Example 9: AI Classification Model for Antibody Therapeutic Design

Methods and systems of the present disclosure are used to develop an AI classification model for antibody therapeutic design.

Antibodies are currently the largest class of biologic-based therapeutics due to their binding versatility, with five antibody therapeutics ranked in the top 10 blockbuster drugs. Continued utilization of antibody therapeutics may rely on more efficient ways to discover and develop these molecules. Antibody therapeutics discovery may require improved methods for lead candidate discovery and engineering. For example, an important biological variable to be efficiently assessed in the antibody discovery process is to identify whether the designed antibody is a strong-binder, weak-binder, or non-binder with respect to its target. Other methods may be laboratory-based, which may incur a lengthy and costly process; however, informatics methods may be used for a fast and more efficient antibody discovery process.

Using methods and systems of the present disclosure, a deep learning-based model was developed to design new antibody sequences against a novel antigenic target. The deep learning model offers significant advantages over other models, such as its unique ability to rapidly and accurately design the third complementarity determining regions in the heavy chain (CDR-H3) sequences (e.g., when the input data is scarce for a novel or existing target). The AI technology was used toward designing new antibodies against SARS-CoV-2 and developing an antibody CDR-H3 hit-generation tool. The extracted key features offered by the deep learning model were further utilized to analyze antibody sequences, and demonstrate antibodies' specificity and cross-reactivity to one or multiple targets for lead identification purposes.

Methods and systems of the present disclosure are used to enable fast and large-scale yet detailed characterization of antibody binding, which may be applicable to in-silico designed and engineered libraries and panels of natural antibodies to understand their binding capabilities better. The approaches are enabled by integration of a powerful deep learning-based model to design and analyze antibody binding capabilities.

First, methods and systems of the present disclosure are used to develop a deep learning-based algorithm to classify antibodies based on their binding capabilities. For example, a deep neural network (DNN) classification model is developed to analyze the binding capability of antibodies with respect to a specific target. If needed, a binding index is incorporated into the model for the most efficient analysis. The binding index comprises a defined range for strong-binder, weak-binder, and non-binder. The DNN classification model is first trained using the antibody CDR-H3 sequences with binder range labels. As an example, a cancer (e.g., breast cancer) antibody library may be used.

Second, methods and systems of the present disclosure are used to develop an automated method to evaluate the DNN classifier performance and to validate antibody binders. An automated method is established to evaluate the DNN model's performance. Evaluation metrics of ROC, AUC, accuracy, specificity, and sensitivity are incorporated into the automated method. The performance of the DNN classifier is compared to a few baseline models using evaluation metrics provided by the automated method. For a systematic analysis on the validity of antibody binder, an automated method is used to compare the input data with those identified by the DNN classifier.

Methods and systems of the present disclosure are used to develop an AI-based model for rapid antibody analysis, which can enhance the antibody lead identification at the earliest stage of the discovery process. As a result, the model can substantially reduce the time and the cost of antibody therapeutics discovery and development, and minimize clinical failure risk for antibody therapeutics.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-117. (canceled)

118. A computer-implemented method for generating a set of candidate variant amino acid sequences of an antibody, a nanobody, or a fragment thereof, having binding ability to protein targets, comprising:
(a) obtaining a set of seed amino acid sequences; and
(b) processing the set of seed amino acid sequences using a first trained machine learning algorithm to generate the set of novel candidate variant amino acid sequences,
wherein the first trained machine learning algorithm is trained with the first training data comprising a set of training amino acid sequences for the protein targets,
wherein the first trained machine learning algorithm is further trained through a transfer learning method using a second trained machine learning algorithm, wherein the second trained machine learning algorithm is trained with second training data comprising a set of training amino acid sequences for a second protein target, wherein the second protein target is different from the first protein targets.

119. The method of claim 118, wherein the set of seed amino acid sequences comprises antibody variable regions (Fvs).

120. The method of claim 119, wherein the Fvs comprise complementarity determining regions (CDRs).

121. The method of claim 119, wherein the Fvs comprise heavy chains (VH) or light chains (VL).

122. The method of claim 119, wherein the Fvs comprise frameworks (FWRs).

123. The method of claim 118, wherein the protein targets comprise at least a portion of a target antigen.

124. The method of claim 118, wherein the set of training amino acid sequences for the second protein target has a smaller number of training data than the set of training amino acid sequences for the first protein targets.

125. The method of claim 118, further comprises, prior to (b), pre-processing the set of seed amino acid sequences (i) to have the same sequence length or (ii) at least in part by generating a numerical representation of the set of seed amino acid sequences.

126. The method of claim 125, wherein the numerical representation comprises a matrix, wherein the matrix comprises a one-hot encoding and/or an embedding pre-processing of (i) the set of amino acid sequences or (ii) amino acid bio-physicochemical values.

127. The method of claim 126, wherein the amino acid bio-physicochemical values are selected from isoelectric point, volume, hydrophobicity, solubility, stability, charge, solvent-accessible surface area (SASA), Immunogenicity, and humanness score.

128. The method of claim 118, wherein the first trained machine learning algorithm or the second trained machine learning algorithm comprises a deep learning model.

129. The method of claim 128, wherein the deep learning model comprises a deep generative AI model, wherein the deep generative AI model comprises at least one of a generative adversarial network (GAN), an autoencoder (AE), a variational autoencoder (VAE), and a residual neural network (Resnet), and a Reinforcement Learning (RL).

130. The method of claim 118, wherein the first trained machine learning algorithm or the second trained machine learning algorithm comprises an encoder and a decoder.

131. The method of claim 130, further comprising performing, using the encoder, a dimensionality reduction of the set of seed amino acid sequences.

132. The method of claim 131, further comprising extracting a set of features associated with the set of seed amino acid sequences generated by the encoder, and reconstructing the set of features using a decoder thereby generating new candidate variant amino acid sequences.

133. The method of claim 131, further comprising clustering features of the set of seed amino acid sequences based on sequence similarity or sequence diversity.

134. The method of claim 133, wherein the clustering comprises generating a two-dimensional plot indicative of single specificity or multi-specificity of the set of seed amino acid sequences.

135. The method of claim 118, wherein the first trained machine learning algorithm and the second trained machine learning algorithm are trained without the use of structural data.

136. The method of claim 118, further comprising processing the set of generated candidate variant amino acid sequences to determine a therapeutic property selected from single specificity, multiple specificity, single cross-reactivity, and multiple cross-reactivity.

137. The method of claim 118, further comprising processing the set of generated candidate variant amino acid sequences to determine molecular characteristics of the set of generated candidate variant amino acid sequences.

138. The method of claim 118, wherein the molecular characteristics comprise at least one of amino acid distribution, amino acid frequency, amino acid length distribution, sequence similarity, sequence diversity, total charge distribution, hydrophobicity, net charge, stability, solubility, isoelectric point, solvent accessible surface area (SASA), binding affinity, affinity maturation, epitope-paratrope interaction, epitope prediction, humaneness, humanization, developability, manufacturability, half-life, pharmacokinetic, yield, aggregation, function, clearance, viscosity, immunogenicity, single-target specificity, multi-target specificity, single-target cross-reactivity, and multi-target cross-reactivity.

139. The method of claim 138, further comprising determining sequence similarity between the set of generated candidate variant amino acid sequences and the set of seed amino acid sequences.

140. The method of claim 138, further comprising determining sequence diversity between the set of generated candidate variant amino acid sequences and the set of seed amino acid sequences.

141. The method of claim 138, further comprising determining specificity or binding affinity of the set of generated candidate variant amino acid sequences to the protein target.

142. The method of claim 138, further comprising (i) selecting or ranking at least one of the set of generated candidate variant amino acid sequences based at least in part on having desired molecular characteristics, or (ii) filtering out at least one of the set of generated candidate variant amino acid sequences having undesired molecular characteristics.

143. The method of claim 118, wherein the set of generated candidate variant amino acid sequences correspond to an antibody, a nanobody, or a fragment thereof.

144. The method of claim 118, further comprising predicting a humanized antibody, nanobody, or fragment variable region based at least in part on the set of generated candidate variant amino acid sequences.

145. A computer-implemented method for generating a set of candidate variant amino acid sequences of an antibody, a nanobody, or a fragment thereof, having binding ability to a protein target, comprising:
(a) obtaining a set of seed amino acid sequences; and
(b) processing the set of seed amino acid sequences using a trained machine learning algorithm to generate the set of candidate variant amino acid sequences, wherein the trained machine learning algorithm comprises at least one of a generative adversarial network (GAN) and Reinforcement Learning (RL).
